SEQUENCE LISTING (58 sequences)

SEQ ID NO: 1 - DNA, 17 bp, synthetic construct
  source 1..17: mol_type = other DNA
gggagattcgtctccca

SEQ ID NO: 2 - DNA, 67 bp, Hepatitis D virus
  source 1..67: mol_type = unassigned DNA
ttcgggtcggcatggcatctccacctcctcgcggtccgacctgggctacttcggtaggctaagggag

SEQ ID NO: 3 - DNA, 67 bp, synthetic construct
  source 1..67: mol_type = other DNA
ttcgggtcggcatggcatctccacctcctcgcggtccgacctgggctacttcggtaggttaagggag

SEQ ID NO: 4 - DNA, 45 bp, synthetic construct
  source 1..45: mol_type = other DNA
ctataaccccttggggcctctaaacgggtcttgaggggttttttg

SEQ ID NO: 5 - DNA, 45 bp, synthetic construct
  source 1..45: mol_type = other DNA
atataaccccttggggcctctaaacgggtcttgaggggttttttg

SEQ ID NO: 6 - DNA, 128 bp, synthetic construct
  source 1..128: mol_type = other DNA
ttctaatacgactcactatagggagattcgtctcccaatcaataacacacatactataaccccttggggcctctaaacgggtcttgaggggttttttgctgaaaggaggaactatatccggatatccc

SEQ ID NO: 7 - DNA, 128 bp, synthetic construct
  source 1..128: mol_type = other DNA
ttctaatacgactcactatagggagattcgtctcccatcacttcacaacatcactataaccccttggggcctctaaacgggtcttgaggggttttttgctgaaaggaggaactatatccggatatccc

SEQ ID NO: 8 - DNA, 128 bp, synthetic construct
  source 1..128: mol_type = other DNA
ttctaatacgactcactatagggagattcgtctcccacacattcaatacatcactataaccccttggggcctctaaacgggtcttgaggggttttttgctgaaaggaggaactatatccggatatccc

SEQ ID NO: 9 - DNA, 128 bp, synthetic construct
  source 1..128: mol_type = other DNA
ttctaatacgactcactatagggagattcgtctcccaaccactaaacacatcactataaccccttggggcctctaaacgggtcttgaggggttttttgctgaaaggaggaactatatccggatatccc

SEQ ID NO: 10 - DNA, 128 bp, synthetic construct
  source 1..128: mol_type = other DNA
ttctaatacgactcactatagggagattcgtctccctaccatactacacatcactataaccccttggggcctctaaacgggtcttgaggggttttttgctgaaaggaggaactatatccggatatccc

SEQ ID NO: 11 - DNA, 128 bp, synthetic construct
  source 1..128: mol_type = other DNA
ttctaatacgactcactatagggagattcgtctcccaattaactcacttcacactttaaccccttggggcctctaaacgggtcttgaggggttttttgctgaaaggaggaactatatccggatatccc

SEQ ID NO: 12 - DNA, 126 bp, synthetic construct
  source 1..126: mol_type = other DNA
ttctaatacgactcactatagggagattcgtctcccatcacttcacaacatatataaccccttggggcctctaaacgggtcttgaggggttttttgctgaaaggaggaactatatccggatatccc

SEQ ID NO: 13 - DNA, 130 bp, synthetic construct
  source 1..130: mol_type = other DNA
ttctaatacgactcactatagggagattcgtctcccatcacttcacaacatcataatataaccccttggggcctctaaacgggtcttgaggggttttttgctgaaaggaggaactatatccggatatccc

SEQ ID NO: 14 - DNA, 132 bp, synthetic construct
  source 1..132: mol_type = other DNA
ttctaatacgactcactatagggagattcgtctcccatcacttcacaacatcatactatataaccccttggggcctctaaacgggtcttgaggggttttttgctgaaaggaggaactatatccggatatccc

SEQ ID NO: 15 - DNA, 128 bp, synthetic construct
  source 1..128: mol_type = other DNA
ttctaatacgactcactatagggagattcgtctcccaatcaataacaacatcactataaccccttggggcctctaaacgggtcttgaggggttttttgctgaaaggaggaactatatccggatatccc

SEQ ID NO: 16 - DNA, 128 bp, synthetic construct
  source 1..128: mol_type = other DNA
ttctaatacgactcactatagggagattcgtctcccatcacttcacacacatactataaccccttggggcctctaaacgggtcttgaggggttttttgctgaaaggaggaactatatccggatatccc

SEQ ID NO: 17 - DNA, 197 bp, synthetic construct
  source 1..197: mol_type = other DNA
ttctaatacgactcactatagggagattcgtctcccaacattacataatcaaatacatacatattcgggtcggcatggcatctccacctcctcgcggtccgacctgggctacttcggtaggttaagggagtgatgttgtgaagttagttaatctataaccccttggggcctctaaacgggtcttgaggggttttttg

SEQ ID NO: 18 - DNA, 197 bp, synthetic construct
  source 1..197: mol_type = other DNA
ttctaatacgactcactatagggagattcgtctcccaccacatactaattaacttacttcacattcgggtcggcatggcatctccacctcctcgcggtccgacctgggctacttcggtaggttaagggagtgatgttgtgaagttagttaatctataaccccttggggcctctaaacgggtcttgaggggttttttg

SEQ ID NO: 19 - DNA, 203 bp, synthetic construct
  source 1..203: mol_type = other DNA
ttctaatacgactcactatagggagattcgtctcccactacatccacatactaattaacttacttcacattcgggtcggcatggcatctccacctcctcgcggtccgacctgggctacttcggtaggttaagggagtgatgttgtgaagtgagttaatctataaccccttggggcctctaaacgggtcttgaggggttttttg

SEQ ID NO: 20 - DNA, 198 bp, synthetic construct
  source 1..198: mol_type = other DNA
ttctaatacgactcactatagggagattcgtctcccaccacatactaattaacttacttcacattcgggtcggcatggcatctccacctcctcgcggtccgacctgggctacttcggtaggctaagggagatacactcactatcactactcattcataaccccttggggcctctaaacgggtcttgaggggttttttg

SEQ ID NO: 21 - DNA, 204 bp, synthetic construct
  source 1..204: mol_type = other DNA
ttctaatacgactcactatagggagattcgtctcccactacatccacatactaattaacttacttcacattcgggtcggcatggcatctccacctcctcgcggtccgacctgggctacttcggtaggctaagggagatacactcactatcactactcattcataaccccttggggcctctaaacgggtcttgaggggttttttg

SEQ ID NO: 22 - DNA, 203 bp, synthetic construct
  source 1..203: mol_type = other DNA
ttctaatacgactcactatagggagattcgtctcccaccacatactaattaacttacttcacataccctcacagtccagtgattcgtcactgagaagtgaaccagagaaacacacgttgtggtatattacctggtctgatgttgtgaagtgagttaatctataaccccttggggcctctaaacgggtcttgaggggttttttg

SEQ ID NO: 23 - DNA, 171 bp, synthetic construct
  source 1..171: mol_type = other DNA
ttctaatacgactcactatagggagattcgtctcccattcgggtcggcatggcatctccacctcctcgcggtccgacctgggctacttcggtaggctaagggagtgatgttgtgaagtgagttaatctataaccccttggggcctctaaacgggtcttgaggggttttttg

SEQ ID NO: 24 - DNA, 197 bp, synthetic construct
  source 1..197: mol_type = other DNA
ttctaatacgactcactatagggagattcgtctcccaccacatactaattaacttacttcacattcgggtcggcatggcatctccacctcctcgcggtccgacctgggctacttcggtaggctaagggagtgatgttgtgaagtgagttaatctataaccccttggggcctctaaacgggtcttgaggggttttttg

SEQ ID NO: 25 - DNA, 203 bp, synthetic construct
  source 1..203: mol_type = other DNA
ttctaatacgactcactatagggagattcgtctcccactacatccacatactaattaacttacttcacattcgggtcggcatggcatctccacctcctcgcggtccgacctgggctacttcggtaggctaagggagtgatgttgtgaagtgagttaatctataaccccttggggcctctaaacgggtcttgaggggttttttg

SEQ ID NO: 26 - DNA, 197 bp, synthetic construct
  source 1..197: mol_type = other DNA
ttctaatacgactcactatagggagattcgtctcccaccacatactaattaaccatattcaatttcgggtcggcatggcatctccacctcctcgcggtccgacctgggctacttcggtaggctaagggagtgatgtattgaatgtggttaatctataaccccttggggcctctaaacgggtcttgaggggttttttg

SEQ ID NO: 27 - DNA, 203 bp, synthetic construct
  source 1..203: mol_type = other DNA
ttctaatacgactcactatagggagattcgtctcccactacatccacatactaattaaccatattcaatttcgggtcggcatggcatctccacctcctcgcggtccgacctgggctacttcggtaggctaagggagtgatgtattgaatgtggttaatctataaccccttggggcctctaaacgggtcttgaggggttttttg

SEQ ID NO: 28 - DNA, 203 bp, synthetic construct
  source 1..203: mol_type = other DNA
ttctaatacgactcactatagggagattcgtctcccactacatccacatactaattaacactactaaacttcgggtcggcatggcatctccacctcctcgcggtccgacctgggctacttcggtaggctaagggagtgatgtgtttagtggtgttaatctataaccccttggggcctctaaacgggtcttgaggggttttttg

SEQ ID NO: 29 - DNA, 203 bp, synthetic construct
  source 1..203: mol_type = other DNA
ttctaatacgactcactatagggagattcgtctcccactacatccacatactaattaacactatactacttcgggtcggcatggcatctccacctcctcgcggtccgacctgggctacttcggtaggctaagggagtgatgtgtagtatggtgttaatctataaccccttggggcctctaaacgggtcttgaggggttttttg

SEQ ID NO: 30 - DNA, 197 bp, synthetic construct
  source 1..197: mol_type = other DNA
ttctaatacgactcactatagggagattcgtctcccatcacttcacaacatcaactactaaacttcgggtcggcatggcatctccacctcctcgcggtccgacctgggctacttcggtaggctaagggagtgatgtgtttagtggttgatgtctataaccccttggggcctctaaacgggtcttgaggggttttttg

SEQ ID NO: 31 - DNA, 197 bp, synthetic construct
  source 1..197: mol_type = other DNA
ttctaatacgactcactatagggagattcgtctcccatcacttcacaacatcaactatactacttcgggtcggcatggcatctccacctcctcgcggtccgacctgggctacttcggtaggctaagggagtgatgtgtagtatggttgatgtctataaccccttggggcctctaaacgggtcttgaggggttttttg

SEQ ID NO: 32 - DNA, 197 bp, synthetic construct
  source 1..197: mol_type = other DNA
ttctaatacgactcactatagggagattcgtctcccaaccatactacacatcaactactaaacttcgggtcggcatggcatctccacctcctcgcggtccgacctgggctacttcggtaggctaagggagtgatgtgtttagtggttgatgtaattaaccccttggggcctctaaacgggtcttgaggggttttttg

SEQ ID NO: 33 - DNA, 197 bp, synthetic construct
  source 1..197: mol_type = other DNA
ttctaatacgactcactatagggagattcgtctcccaaccactaaacacatcacatattcaatttcgggtcggcatggcatctccacctcctcgcggtccgacctgggctacttcggtaggctaagggagtgatgtattgaatgtgtgatgtaattaaccccttggggcctctaaacgggtcttgaggggttttttg

SEQ ID NO: 34 - DNA, 232 bp, synthetic construct
  source 1..232: mol_type = other DNA
ttctaatacgactcactatagggagattcgtctcccactacatccacatactaattaacttacttcacataatacattcaatttcgggtcggcatggcatctccacctcctcgcggtccgacctgggctacttcggtaggctaagggagtgatgtattgaatgtgtgatgttgtgaagtgagttaatctataaccccttggggcctctaaacgggtcttgaggggttttttg

SEQ ID NO: 35 - DNA, 187 bp, synthetic construct
  source 1..187: mol_type = other DNA
ttctaatacgactcactatagggagattcgtctcccattcgggtcggcatggcatctccacctcctcgcggtccgacctgggctacttcggtaggctaagggagtgatgtattgaatgtgtgatgttgtgaagtgagttaatctataaccccttggggcctctaaacgggtcttgaggggttttttg

SEQ ID NO: 36 - DNA, 226 bp, synthetic construct
  source 1..226: mol_type = other DNA
ttctaatacgactcactatagggagattcgtctcccatcacttcacaacatcaactactaaactaaactatactacttcgggtcggcatggcatctccacctcctcgcggtccgacctgggctacttcggtaggctaagggagtgatgtgtagtatggttgatgtgtttagtggttgatgtctataaccccttggggcctctaaacgggtcttgaggggttttttg

SEQ ID NO: 37 - DNA, 232 bp, synthetic construct
  source 1..232: mol_type = other DNA
ttctaatacgactcactatagggagattcgtctcccactacatccacatactaattaacactactaaactaaactatactacttcgggtcggcatggcatctccacctcctcgcggtccgacctgggctacttcggtaggctaagggagtgatgtgtagtatggttgatgtgtttagtggtgttaatctataaccccttggggcctctaaacgggtcttgaggggttttttg

SEQ ID NO: 38 - DNA, 227 bp, synthetic construct
  source 1..227: mol_type = other DNA
ttctaatacgactcactatagggagattcgtctcccaccacatactaattaacttacttcacattcacatattcaatttcgggtcggcatggcatctccacctcctcgcggtccgacctgggctacttcggtaggctaagggagtgatgtattgaatgtgtgatgttgtgaagtgagttaatctataaccccttggggcctctaaacgggtcttgaggggttttttg

SEQ ID NO: 39 - DNA, 226 bp, synthetic construct
  source 1..226: mol_type = other DNA
ttctaatacgactcactatagggagattcgtctcccaccacatactaattaacttacttcacaccatacattcaatttcgggtcggcatggcatctccacctcctcgcggtccgacctgggctacttcggtaggctaagggagtgatgtattgaatgtgtgatgttgtgaagtgagttaatctataaccccttggggcctctaaacgggtcttgaggggttttttg

SEQ ID NO: 40 - DNA, 226 bp, synthetic construct
  source 1..226: mol_type = other DNA
ttctaatacgactcactatagggagattcgtctcccaccacatactaattaacttacttcacataatacattcaatttcgggtcggcatggcatctccacctcctcgcggtccgacctgggctacttcggtaggctaagggagtgatgtattgaatgtgtgatgttgtgaagtgagttaatctataaccccttggggcctctaaacgggtcttgaggggttttttg

SEQ ID NO: 41 - DNA, 224 bp, synthetic construct
  source 1..224: mol_type = other DNA
ttctaatacgactcactatagggagattcgtctcccaccacatactaattaacttacttcacaccatattcaatttcgggtcggcatggcatctccacctcctcgcggtccgacctgggctacttcggtaggctaagggagtgatgtattgaatgtgtgatgttgtgaagtgagttaatctataaccccttggggcctctaaacgggtcttgaggggttttttg

SEQ ID NO: 42 - DNA, 197 bp, synthetic construct
  source 1..197: mol_type = other DNA
ttctaatacgactcactatagggagattcgtctcccatcacttcacaacatcaactatactacttcgggtcggcatggcatctccacctcctcgcggtccgacctgggctacttcggtaggctaagggagtacactcactatcactactcattcataaccccttggggcctctaaacgggtcttgaggggttttttg

SEQ ID NO: 43 - DNA, 191 bp, synthetic construct
  source 1..191: mol_type = other DNA
ttctaatacgactcactatagggagattcgtctcccatcacttcacaacatcaactatactacttcgggtcggcatggcatctccacctcctcgcggtccgacctgggctacttcggtaggttaagggagtgatgtgtagtatggtctataaccccttggggcctctaaacgggtcttgaggggttttttg

SEQ ID NO: 44 - DNA, 205 bp, synthetic construct
  source 1..205: mol_type = other DNA
ttctaatacgactcactatagggagattcgtctcccactacatccacatactaattaacttacttcacattcgggtcggcatggcatctccacctcctcgcggtccgacctgggctacttcggtaggctaagggagtatgatgttgtgaagtgagttaatctataaccccttggggcctctaaacgggtcttgaggggttttttg

SEQ ID NO: 45 - DNA, 207 bp, synthetic construct
  source 1..207: mol_type = other DNA
ttctaatacgactcactatagggagattcgtctcccactacatccacatactaattaacttacttcacattcgggtcggcatggcatctccacctcctcgcggtccgacctgggctacttcggtaggctaagggagagtatgatgttgtgaagtgagttaatctataaccccttggggcctctaaacgggtcttgaggggttttttg

SEQ ID NO: 46 - DNA, 209 bp, synthetic construct
  source 1..209: mol_type = other DNA
ttctaatacgactcactatagggagattcgtctcccactacatccacatactaattaacttacttcacattcgggtcggcatggcatctccacctcctcgcggtccgacctgggctacttcggtaggctaagggagagagtatgatgttgtgaagtgagttaatctataaccccttggggcctctaaacgggtcttgaggggttttttg

SEQ ID NO: 47 - DNA, 25 bp, synthetic construct
  source 1..25: mol_type = other DNA
ttctaatacgactcactatagggag

SEQ ID NO: 48 - DNA, 26 bp, synthetic construct
  source 1..26: mol_type = other DNA
caaaaaacccctcaagacccgtttag

SEQ ID NO: 49 - DNA, 66 bp, synthetic construct
  source 1..66: mol_type = other DNA
ttctaatacgactcactatagggagattcgtctcccaccacatactaattaacttacttcacattc

SEQ ID NO: 50 - DNA, 66 bp, synthetic construct
  source 1..66: mol_type = other DNA
gaatgtgaagtaagttaattagtatgtggtgggagacgaatctccctatagtgagtcgtattagaa

SEQ ID NO: 51 - DNA, 72 bp, synthetic construct
  source 1..72: mol_type = other DNA
ttctaatacgactcactatagggagattcgtctcccactacatccacatactaattaacttacttcacattc

SEQ ID NO: 52 - DNA, 72 bp, synthetic construct
  source 1..72: mol_type = other DNA
gaatgtgaagtaagttaattagtatgtggatgtagtgggagacgaatctccctatagtgagtcgtattagaa

SEQ ID NO: 53 - DNA, 16 bp, synthetic construct
  source 1..16: mol_type = other DNA
ctacatccacatacta

SEQ ID NO: 54 - DNA, 22 bp, synthetic construct
  source 1..22: mol_type = other DNA
gttaattagtatgtggatgtag

SEQ ID NO: 55 - RNA, 78 nt, synthetic construct
  source 1..78: mol_type = other RNA
gggagattcgtctcccatcacttcacaacatcaatataaccccttggggcctctaaacgggtcttgaggggttttttg

SEQ ID NO: 56 - RNA, 46 nt, synthetic construct
  source 1..46: mol_type = other RNA
gggagattcgtctcccaccacatactaattaacttacttcacattc

SEQ ID NO: 57 - RNA, 131 nt, synthetic construct
  source 1..131: mol_type = other RNA
gggtcggcatggcatctccacctcctcgcggtccgacctgggctacttcggtaggctaagggagtgatgttgtgaagtgagttaatctataaccccttggggcctctaaacgggtcttgaggggttttttg

SEQ ID NO: 58 - skipped

(19) United States
(12) Patent Application Publication
(10) Pub. No.: US 2025/0059598 A1
(43) Pub. Date: Feb. 20, 2025

(54) CTRSD GATE AND PERFORMING CO-TRANSCRIPTIONAL ENCODING

(71) Applicant: Government of the United States of America, as represented by the Secretary of Commerce, Gaithersburg, MD (US)

(72) Inventor: Samuel W. Schaffter, Washington, DC (US)

(21) Appl. No.: 18/720,950

(22) PCT Filed: Dec. 16, 2022
(86) PCT No.: PCT/US2022/053229

§ 371 Date: Jun. 17, 2024

Related U.S. Application Data
(60) Provisional application No. 63/290,457, filed on Dec. 16, 2021.

(51) Int. Cl. C12Q 1/6865

(52) U.S. Cl. CPC C12Q 1/6865

(57) ABSTRACT

A method for the production and use of scalable co-transcriptional RNA strand displacement (ctRSD) circuits using RNA toehold exchange gates is described. The ctRSD circuits described address the limitations of existing DNA-based strand displacement circuits by isothermally producing circuit components via transcription.

### CROSS REFERENCE TO RELATED APPLICATIONS

This application is a U.S. National Phase Application of PCT/US2022/053229 (filed Dec. 16, 2022), which claims the benefit of U.S. Provisional Application No. 63/290,457 (filed Dec. 16, 2021), the disclosures of each of which are hereby incorporated by reference in their entirety.

### STATEMENT REGARDING FEDERALLY SPONSORED RESEARCH

This invention was made with United States Government support from the National Institute of Standards and Technology (NIST), an agency of the United States Department of Commerce. The Government has certain rights in this invention.

### SEQUENCE LISTING

This application contains a Sequence Listing which has been filed electronically in compliance with ST.26 format and is hereby incorporated by reference in its entirety. The Sequence Listing, created on Jun. 26, 2024 is named 21-053US1_Sequence_Listing.xml and is 58 kilobytes in size.

### BRIEF DESCRIPTION OF THE DRAWINGS

The following description cannot be considered limiting in any way. Various objectives, features, and advantages of the disclosed subject matter can be more fully appreciated with reference to the following detailed description of the disclosed subject matter when considered in connection with the following drawings, in which like reference numerals identify like elements.

FIG. 1 shows an output strand 201, according to some embodiments.

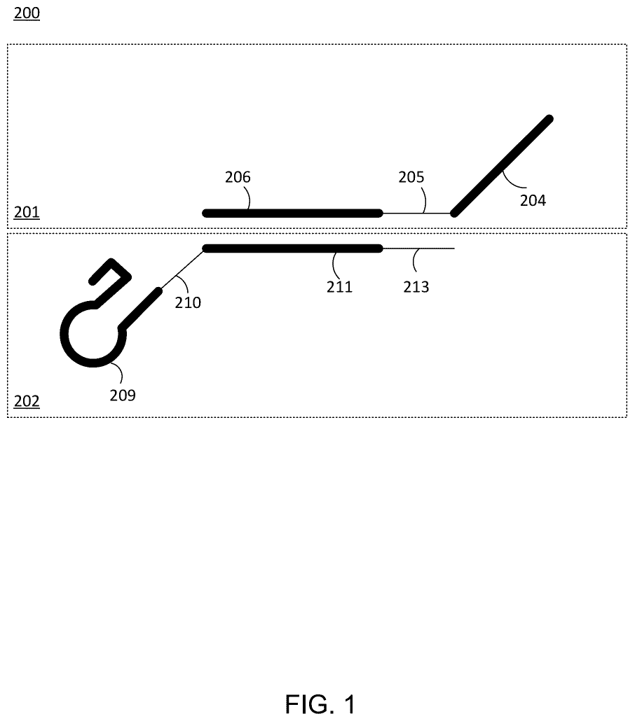

FIG. 2 shows an output strand 201, according to some embodiments.

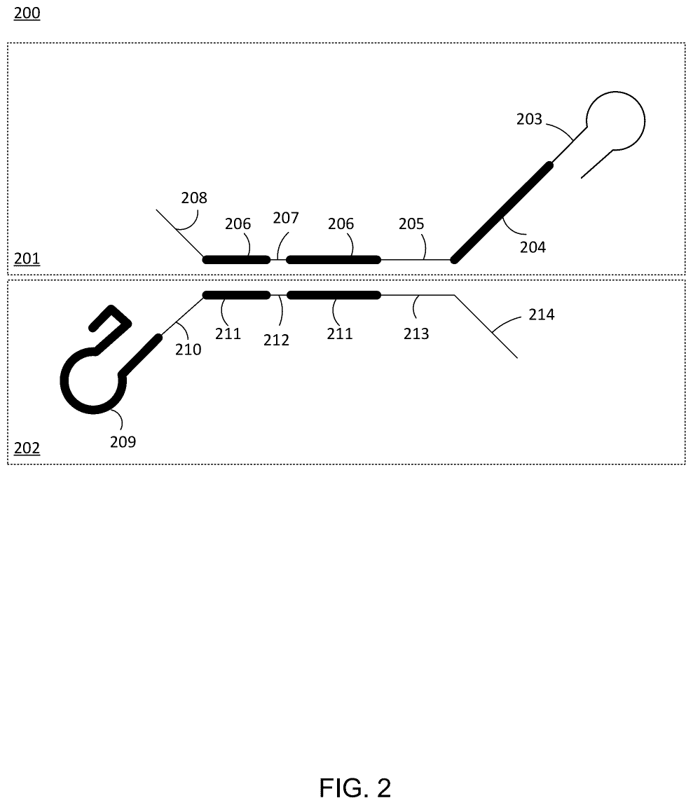

FIG. 3 shows an output strand 201, according to some embodiments.

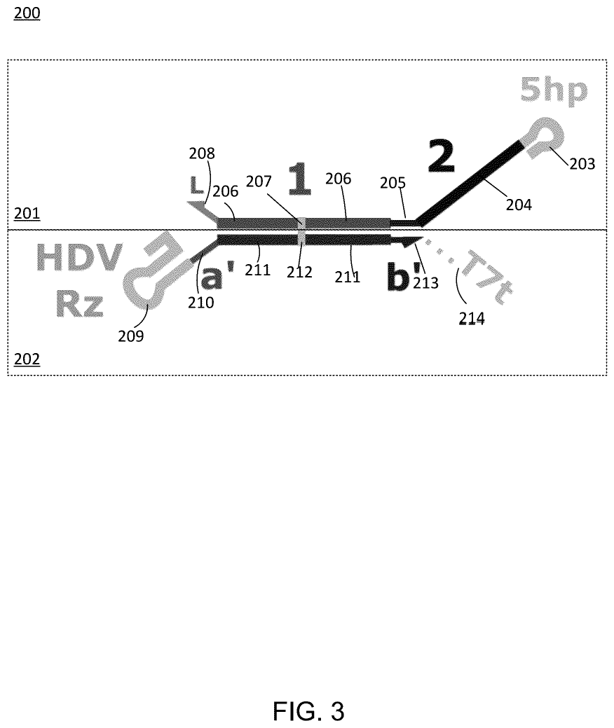

FIG. 4 shows an output strand 201, according to some embodiments.

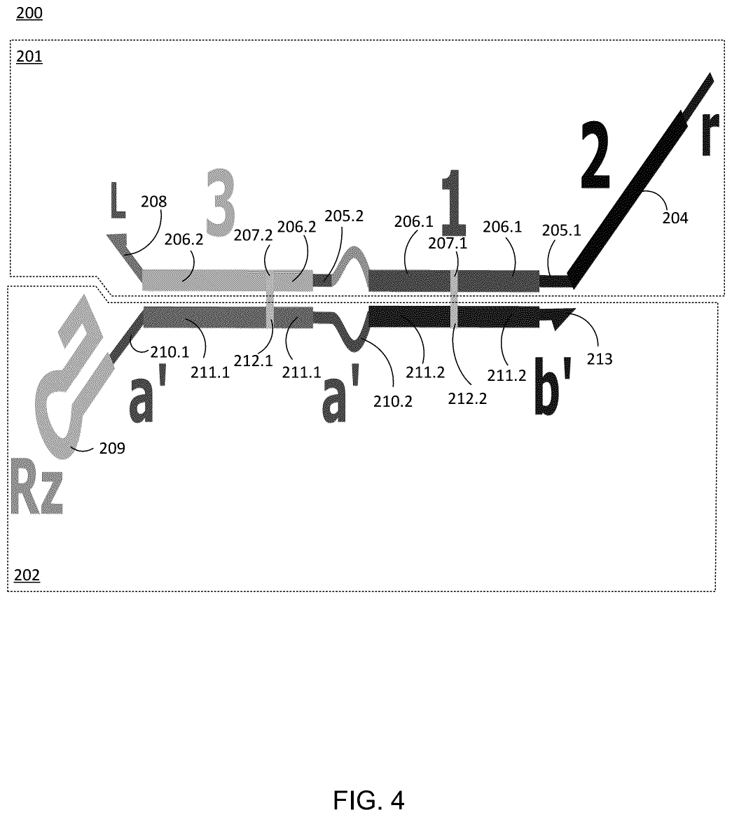

FIG. 5 shows (A) production of a strand displacement product 216 from a reaction between an input template strand 215.1 and a ctRSD gate 200, wherein strand displacement product 216 and a second input template strand 215.2 is produced, and (B) a serial reaction in which a series of input template strands 215 react with various ctRSD gates 200, wherein the serial reaction is propagated by reactant products (e.g., 215.2, . . . , 215.5) formed from prior parent ctRSD gates 200 (e.g., 200.1, 200.2, 200.3, 200.4). It should be appreciated that the serial reaction can propagate for an arbitrary number of layers although only four layers are shown.

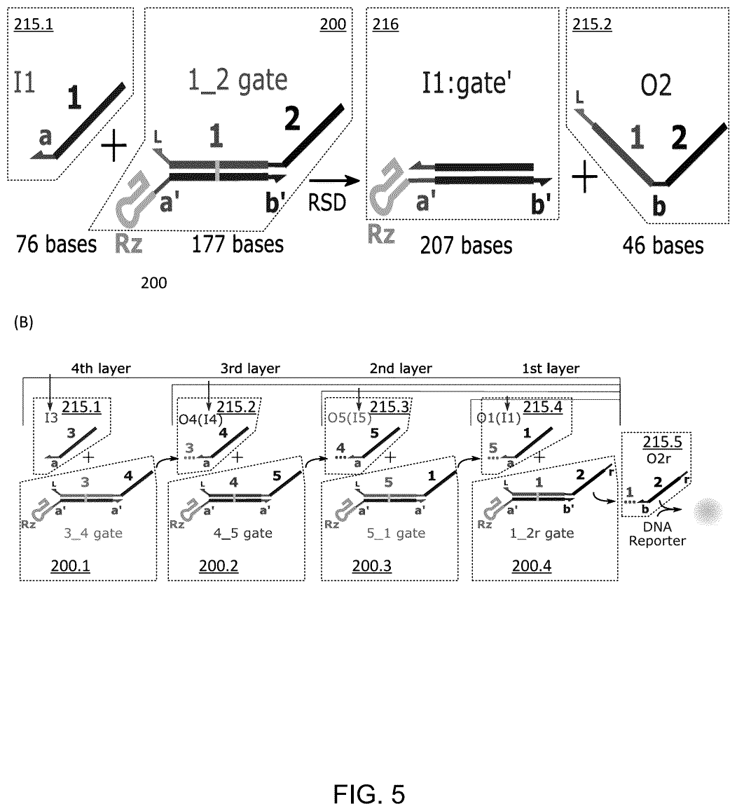

FIG. 6 shows, according to some embodiments, co-transcriptionally encoded RNA strand displacement (ctRSD) circuit design. (A) In DNA strand displacement (DSD), pre-annealed DNA gates are mixed to build a circuit. Strand exchange between the input and gate releases an output. (B) In ctRSD, designed transcription templates produce the RNA components that make up a circuit. DNA and RNA are represented with dashed and solid lines, respectively. Bold letters and numbers represent sequence identity. A prime (′) denotes complementarity. Only one domain of a complementary pair will be shown in subsequent figures for simplicity. The I and O below the gate represent input and output domains, respectively. (C) Transcriptional encoding of ctRSD components. All RNAs possess a 5′ hairpin (5 hp) and a 3′ terminator (T7t). For simplicity, these motifs are omitted elsewhere. The cyan line represents a G-U wobble pairing. The gate contains a self-cleaving ribozyme (HDV Rz) to enable co-transcriptional folding of kinetically trapped gates (D). See FIG. 11 for schematics with sequences. (D) ctRSD gates fold into RNA hairpins that self-cleave to produce reactive dsRNA products. Input and output domains define gate names (e.g. “1_2 gate”). (E) Gel electrophoresis demonstrating gate folding and cleavage (Lane 4, blue box) after 30 min of transcription followed by 30 min of DNase degradation. Lane 1: a transcript that is the same length as the gate but does not fold into a hairpin or cleave (xRz). Lane 2: the 1_2 gate without cleavage (xRz). Lane 3: the gate′ strand (Rz, a′-, 1′-, and b′-domains) alone. Lane 5: separate transcription of the output (O2) and gate′ strands. The 46 base single-stranded O2 strand stained poorly for visualization. See FIG. 12 for control transcript designs.

FIG. 7 shows, according to some embodiments, characterization of strand displacement in ctRSD circuits. (A) Strand displacement between an input and an a ctRSD gate. The 11:gate′ complex is 30 bases longer than the gate. (B) Native RNA gel electrophoresis demonstrating strand displacement in a ctRSD circuit. Lane 1:I1:gate′ complex. Lane 2: 1_2 gate. Lane 3 to lane 7: 25 nmol/L of 1_2 gate template was co-transcribed with 2.5 nmol/L (0.1×) to 50 nmol/L (2×) of 11 template. The 46 base output strand of the gate (O2) was not visible. Transcription proceeded for 30 min, and electrophoresis was conducted 2 h after DNase I addition. Lane 8 to lane 10: I1 and 1_2 gate templates were transcribed separately for 30 min, incubated with DNase I for 30 min, then samples were mixed in equal volumes and incubated at 37° C. for 2 h before electrophoresis. The table below the gel shows the percentage of 1_2 gate in each lane agrees with NUPACK predictions. See FIG. 24 for additional conditions. (C) Schematic of the fluorescent DNA reporter assay to track O2r production. The red dotted line trailing O2r represents the upstream portion of the output strand not involved in downstream reactions. (D) Experimental (solid lines) and simulated (dashed lines) DNA reporter signal during co-transcription of the 1_2r gate with different 11 template concentrations. The gray lines indicate the 1_2r gate co-transcribed with a randomized input sequence (Io) that does interact with the 1_2r gate.

FIG. 8 shows, according to some embodiments, orthogonal ctRSD input and gate sequences. (A) Fluorescent DNA reporter signal during co-transcription of 25 nmol/L gates with orthogonal input domains and 50 nmol/L of the designed input template or the Io template. The dashed lines show the results of the model for the 1_2r gate from FIG. 7D. (B) Native gel electrophoresis results demonstrating orthogonality of the four gate and input sequences. In each gel, 25 nmol/L of a single ctRSD gate was co-transcribed with no input (lane 1) or 50 nmol/L of the (11, 13, 14, or 15) template. Transcription proceeded for 30 min, and electrophoresis was conducted 2 h after degradation of DNA templates with DNase 1. The 12 gate and 3_2 gate samples were analyzed on the same gel. The 4_1 gate and 51 gate samples were analyzed on the same gel. Both gel images were taken with the same setting and were otherwise unmodified.

FIG. 9 shows, according to some embodiments, characterization of ctRSD logic and catalytic amplification elements. (A) A ctRSD OR circuit element. (B) Native gel electrophoresis results for the OR element. Transcription proceeded for 30 min, and electrophoresis was conducted 30 min after DNase I addition. The gate′ strand is from the 1_2 gate. (C) Experimental (solid lines) and simulated (dashed lines) reporter signal during co-transcription of the OR element with different inputs. The trajectories for 11 alone and I3 alone overlap. The 1_2r and 3_2r gates were used in this experiment. (D) A ctRSD AND circuit element. (E) Native RNA gel electrophoresis results for the AND element. Transcription proceeded for 30 min, and electrophoresis was conducted 1 h after DNase I addition. The gate′ is from the 3&1_2r gate. (F) Experimental (solid lines) and simulated (dashed lines) DNA reporter signal during co-transcription of the AND element with different inputs. The trajectories for Io alone and I3 alone overlap. (G) A ctRSD catalytic amplification element. (H and I) Simulated (H) and experimental (1) DNA reporter signal during co-transcription of the 1_2r gate and 11 with (solid lines) and without (dashed lines) the F1 template (1×). For the gel results, gate and input templates were 25 nmol/L and 50 nmol/L, respectively.

FIG. 10 shows, according to some embodiments, characterization of ctRSD cascades. (A) Schematic of one- to four-layer cascades. Green arrows indicate the sole input template included for each cascade layer. The colored dotted lines trailing outputs represent the upstream portion of the output strand not involved in downstream reactions. (B) Experimental (solid lines) and simulated (dashed lines) DNA reporter signal for each layered cascade in (A). Faded lines represent each cascade with the Io template rather than the correct input template. (C to F) Experimental (solid lines) and simulated (dashed lines) reporter signal for each of the logic circuits depicted above the plots. Boxes in (C) to (F) denote the sets of inputs that should result in output release. Overlapping kinetic trajectories are labeled in the plots. In (F), the simulation results for Io, 14, and 15 all overlap with the experimental results for 14.

FIG. 11 shows, according to some embodiments, RNA sequence motifs used to co-transcriptionally encode stand displacement circuit components. (A) The HDV ribozyme (Rz), an internal self-cleaving ribozyme, was added between the output and gate′ strands of ctRSD gates. The red arrow adjacent to the HDV ribozyme indicates the cleavage site. The cytosine highlighted in black is required for catalytic activity and was mutated to uracil in inactive mutants used in this study. Additionally, a T7 RNAP terminator (T7t) sequence was appended to the 3′ end of the gate′ strand of ctRSD gates and to the 3′ end of input sequences. Further, all transcripts were designed with a hairpin motif at their 5′ end (5 hp). This motif possesses the consensus initiation sequence (5′GGGAGA (SEQ ID NO.: 58)) for T7 RNAP. Finally, a G-U wobble base pair was introduced within the double stranded branch migration domain (cyan) to reduce secondary structure in the DNA template, which can interfere with synthesis, and to provide a thermodynamic driving force for strand displacement with inputs that convert the G-U wobble pair to a G-C pair. G-U wobble base pairs are also present in the HDV Rz and T7t sequences. (B) The full sequence schematics of the 11 and the 1_2 gate transcripts.

FIG. 12 shows, according to some embodiments, control RNA transcripts. A black X in the Rz domains indicates a C→U mutation that prevents cleavage from occurring (xRz). Pink cau domains are designed without guanine bases. (A) The 12 ctRSD gate. (B) A control transcript corresponding to the full length of the desired 1_2 gate transcript. In this control, an inactive HDV mutant was used to prevent cleavage, and the 1-, b-, and 2-domains were mutated to prevent the transcript from folding into a hairpin. (C) The 1_2 gate with an inactive HDV mutant to prevent cleavage. In denaturing conditions, this transcript should be the same length as the control transcript in (B). (D) A control transcript corresponding to the gate′ strand of the 12 gate. (E) A control transcript possessing the output sequence of the 1_2 gate, followed by the HDV ribozyme and 22 C, A, or U bases. This control produces an HDV ribozyme cleaved output strand identical to the output strand the 1_2 gate in (A). The product of HDV ribozyme cleavage (Rz- and cau-domains) should also migrate similarly to the control transcript in (D) in agarose gel electrophoresis experiments. (F) A control in which the two strands of the 1_2 gate are produced from separate transcription templates. The O2 transcription template was prepared from two oligos (O2-nt and O2-t in TABLE 1) that were mixed in a 1:1 molar ratio in transcription buffer, heated to 90° C. for 5 min, and then cooled to 20° C. at a rate of 1° C./min. To obtain a 1:1 concentration of O2 RNA and gate′ RNA, 25 nmol/L of the O2 strand template and 18.75 nmol/L of the gate′ strand template were used (FIG. 19).

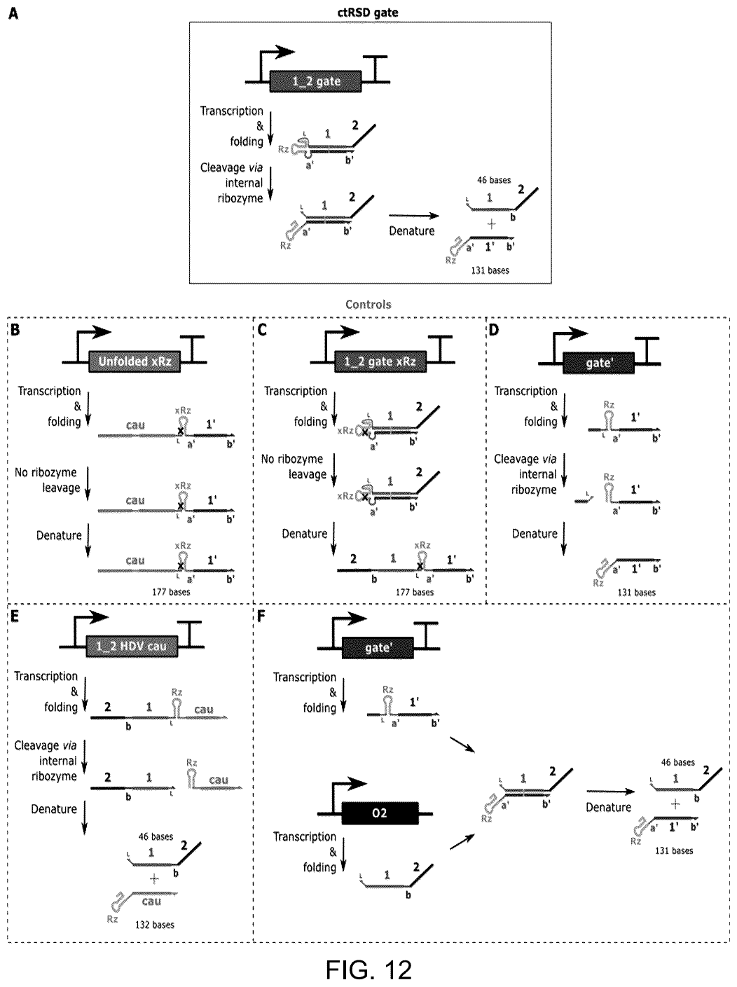

FIG. 13 shows, according to some embodiments, sequence schematics of RSD reactions between 11, 13, 14, or 15 and corresponding ctRSD gates. G-U wobble base pairs are highlighted in cyan. An input reacting with its designed gate turns a G-U wobble pairing to a G-C pairing, providing a thermodynamic driving force for the forward reaction.

FIG. 14 shows, according to some embodiments, sequence schematics of the DNA reporter reacting with the O2 strand (top) or the O2r strand, which is extended to contain the reversible domain of the reporter output (bottom). To be stable at 37° C., the DNA reporter needed a double stranded region of >15 bases, which prompted addition of the r-domain.

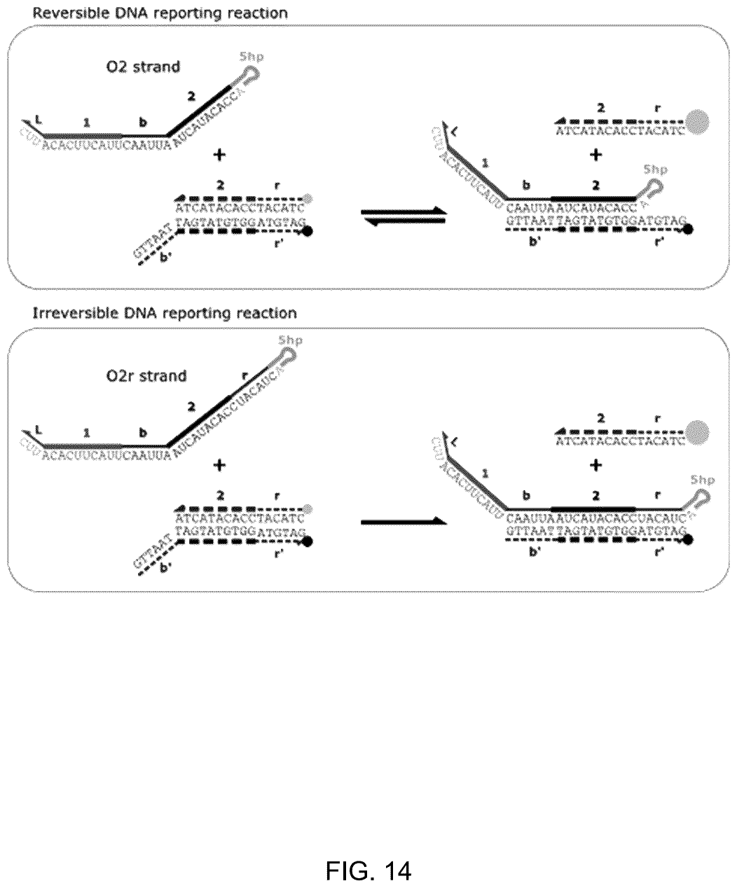

FIG. 15 shows, according to some embodiments, sequence schematics of RSD for 3&1_2 and 5&4_2ctRSD gates. G-U wobble base pairs are highlighted in cyan. An input reacting with its designed gate turns a G-U wobble pairing to a G-C pairing, providing a thermodynamic driving force for the forward reaction.

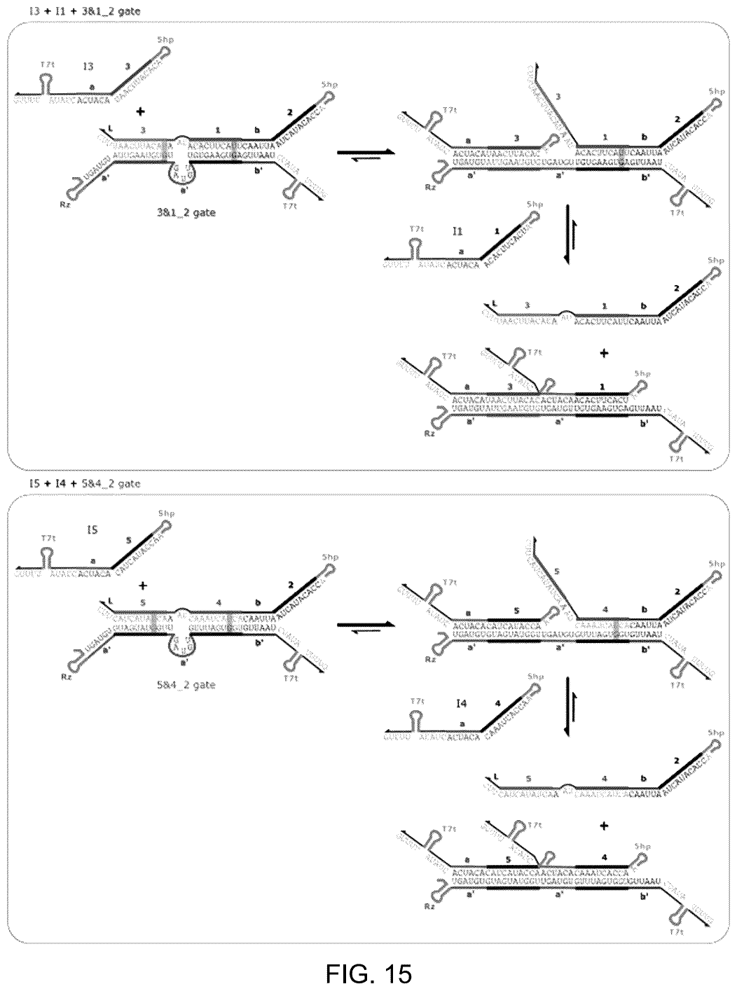

FIG. 16 shows, according to some embodiments, sequence schematics of ctRSD gates and inputs with variable toehold lengths. G-U wobble base pairs are highlighted in cyan. An input reacting with its designed gate turns a G-U wobble pairing to a G-C pairing, providing a thermodynamic driving force for the forward reaction.

FIG. 17 shows, according to some embodiments, overview of options for preparing gates for TMSD circuits. (A) The desired reaction network. 11 releases an output from the 1_2r gate that in turn reacts with a DNA reporter complex to produce a fluorescent signal. The 1_2r gate should not react with the DNA reporter on its own. (B) The typical method for preparing DNA gates for TMSD circuits. The 1_2r gate and the DNA reporter are prepared in separate test tubes and then mixed to make the circuit. Separate preparation ensures the two complexes are kinetically precluded from reacting in the absence of input. (C) A method for preparing ctRSD gates in which separate transcription templates produce the output and gate′ strands of the gate. Here, the two RNA strands should hybridize after transcription to form the gate. However, if this is done alongside the DNA reporter, or any other downstream gates that take O2r as their input, the output strand can also react downstream before it hybridizes to form the gate. This introduces significant leak in the circuit (FIG. 18). (D) A method for preparing ctRSD gates in which a gate is encoded as a hairpin that cleaves into a dsRNA product via an internal self-cleaving ribozyme. In this method, the hairpin rapidly folds during transcription to prevent any downstream reaction in the absence of input.

FIG. 18 shows, according to some embodiments, measuring downstream leak during transcription of the output (O2r) and gate′ strands of the 1_2r gate (FIG. 17C) or transcription of the self-cleaving 1_2r gate hairpin (FIG. 17D). The leak rate was determined by fitting a line (pink dotted lines) to the first 40 min of each reaction. Compared to separate transcription of the two gate strands, transcription of the 1_2r gate with the self-cleaving ribozyme reduces the leak 7.6-fold. To facilitate an irreversible reporting reaction, a 1_2 gate variant with a 6 base 5′ extension (domain r) was used (FIG. 14). Reactions were conducted with 500 nmol/L DNA reporter, 1 U/μL of T7 RNAP, and 25 nmol/L 1_2r gate and O2r templates. Fora 1:1 concentration of O2r RNA and gate′ RNA, 18.75 nmol/L of the gate′ strand template was used (FIG. 19).

FIG. 19 shows, according to some embodiments, titrating the gate′ strand template concentration to achieve equal concentrations of O2r and gate′ transcripts. 25 nmol/L O2r template and 18.75 nmol/L gate′ template yield a 1:1 concentration (blue rectangle). Increasing the gate′ strand template concentration results in excess gate′ transcript. The O2r strand did not stain well. Transcription and native gel electrophoresis were conducted. After transcription, samples were incubated with DNase I for 1 h before gel electrophoresis.

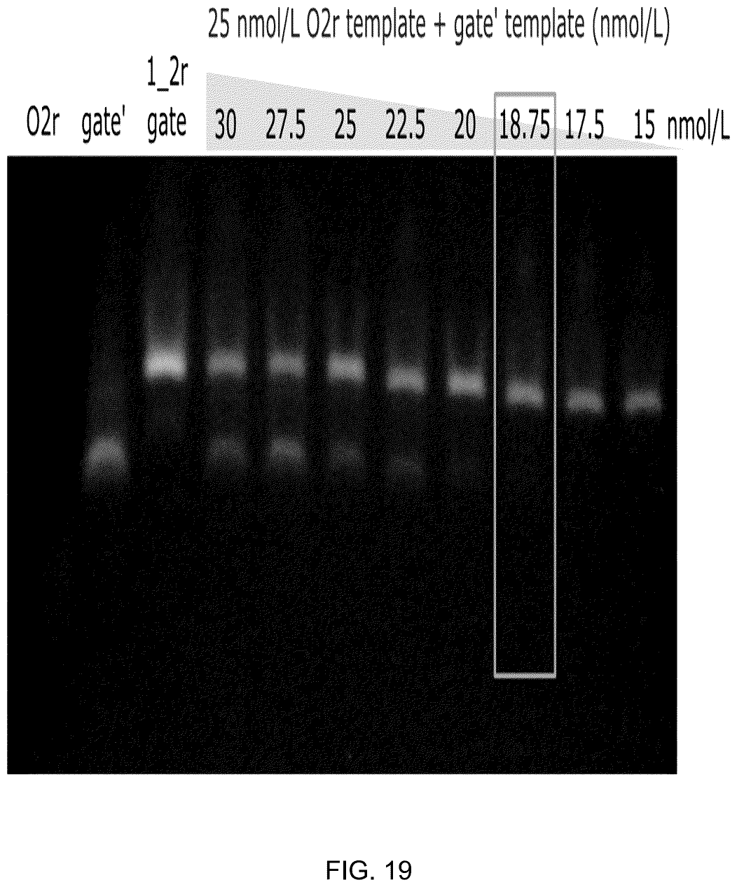

FIG. 20 shows, according to some embodiments, four possible folding pathways for designing ctRSD gates. The two major design choices are the placement of the ssRNA toehold—5′ (A and B) or 3′ (C and D)—and the placement of the self-cleaving ribozyme (Rz)—adjacent (A and C) or opposite (B and D) of the ssRNA toehold. The design in (A) was selected.

FIG. 21 shows, according to some embodiments, designs of ctRSD gates with different self-cleaving ribozymes. Numbers above transcripts indicate domain lengths in nucleotides. In the final cleavage products, the number of bases that must dissociate to expose the toehold are written in gold lettering. The HDV ribozyme does not require any bases to unhybridized after cleavage, making it the best candidate for our ctRSD gate design.

FIG. 22 shows, according to some embodiments, rate and efficiency of internal HDV ribozyme cleavage in ctRSD gates. (A) Schematic of experimental and simulation setup. A DNA template (25 nmol/L) is transcribed to produce the uncleaved ctRSD gate. kₚ is the first order transcription rate constant. The HDV ribozyme can then convert the ctRSD gate into the cleaved product with a first order rate constant k_{rz}. Transcription proceeded for 15 min. DNase I (final concentration 0.23 U/μL, 1.43 mmol/L CaCl₂)) was then added to the samples, and the samples were incubated for varying times prior to the addition of denaturing solution and heating to 90° C. for 5 min. Following denaturing, the samples were immediately run on a 4% EX agarose E-gel (Invitrogen) for 25 min and imaged. For denaturing, the denaturing solution (100% formamide, 36 mmol/L EDTA) was mixed 1:1 by volume with the samples. (B) Denaturing gel results for the experiment described in (A). The first two lanes are controls showing the uncleaved and cleaved RNA products. These samples were incubated with DNase I for 30 min following transcription. The last gel lane shows that all the DNA template is degraded after 2 min with DNase I, suggesting degradation is rapid in our experimental conditions. Almost no detectable uncleaved transcript was left after a 10 min incubation with DNase I. (C) The fraction of cleaved ctRSD gate predicted from a simulation of the experiment depicted in (A). Mass action kinetic equations were used with a kₚ of 0.025 s⁻¹ and different values for k_{rz}. The value of kₚ does not influence the faction of cleaved RNA in simulation. The gray shaded region in the plot indicates the initial 15 min of transcription. At 15 min in the simulations, the DNA template concentration is changed to 0 nmol/L to simulate the addition of DNase 1. Dashed lines indicate the 2 min, 5 min, and 10 min timepoints that were run on the gel in (B). Based on this analysis, we estimate k_{rz} as approximately between (0.25 and 0.35) min⁻¹. This estimated rate is within a factor of 2 of previously published rates for HDV ribozyme self-cleavage in vitro.

FIG. 23 shows, according to some embodiments, denaturing gel of the ctRSD 1_2 gate with the hepatitis delta virus (HDV) or the hairpin (HP) ribozyme. Most of the HP ribozyme transcript does not cleave. In vitro transcription reactions were conducted for 1 h, followed by DNase I digestion (Methods). Samples were mixed with an equal volume of Gel Loading Buffer II (Invitrogen), heated to 80° C. for 5 min, and subsequently run on 4% EX agarose E-gels for 30 min. The 1_2 gate xRz and 1_2 HDV cau controls, which represent the full length uncleaved and cleaved products, respectively, are depicted in FIG. 12. All samples were analyzed on the same gel but lanes between the HDV Rz and HP Rz samples were removed because they were not related to this experiment.

FIG. 24 shows, according to some embodiments, native RNA gel electrophoresis demonstrating RNA strand displacement with ctRSD components. In lane 1 to lane 5, the 11 and 1_2 gate templates were transcribed separately for 30 min and subsequently incubated with DNase I for 30 min. The samples were then mixed and incubated at 37° C. for 2 h before electrophoresis. 25 nmol/L of the 1_2 gate template and 25 nmol/L of the 11 template were transcribed separately and, following template degradation, mixed as follows. The 11 (1×) sample consisted of 5 μL of gate transcript, 5 μL of input transcript, and 10 μL of transcription solution without any template. The 11 (2×) sample consisted of 5 μL of gate transcript, 10 μL of input transcript, and 5 μL of transcription solution without any template. The 11 (3×) sample consisted of 5 μL of gate transcript and 15 μL of input transcript. 20 μL of each sample was loaded into the gel. For lane 6 to lane 10, 25 nmol/L of gate template was transcribed alongside either (25 nmol/L (1×), 50 nmol/L (2×), or 75 nmol/L (3×)) input template. We assume the gate 1_2 gate was folded in lanes (2 to 5) prior to the addition of I1, suggesting the interaction between the input and the gate is the result of strand displacement.

FIG. 25 shows, according to some embodiments, the toehold (a) and branch migration (1) domains are required for strand displacement in ctRSD circuits. (A) Schematics of inputs with scrambled toehold (I1×), scrambled branch migration (Ioa), or scrambled toehold and branch migration (Io) domains. (B) Native RNA gel electrophoresis of the 12 gate with different inputs. Gate and inputs were either transcribed together (gray) or transcribed separately and then mixed (blue). Lanes 1 and 2 are controls. Lane 1:I1:gate′ complex. Lane 2: 1_2 gate. For the co-transcription experiments, the gate and input templates concentrations were 25 nmol/L. Transcription proceeded for 30 min, and electrophoresis was conducted 2 h after DNase I addition. For the separate transcription experiments the gate and input template concentrations were 50 nmol/L prior to mixing. Gate and input samples were transcribed separately for 30 min, incubated with DNase I for 30 min, and then mixed in equal volumes and incubated at 37° C. for 2 h before electrophoresis.

FIG. 26 shows, according to some embodiments, results for experimentally testing RNA strand displacement between 11 and a ctRSD gate with a DNA reporter assay. (A) Experimental set up. The 1_2r gate template (50 nmol/L) and the 11 template (100 nmol/L) were transcribed separately with 1 U/μL T7 RNAP for (0.5, 1, or 2) h. After each transcription timepoint, the samples were heated to 65° C. for 30 min to denature T7 RNAP. After the heat denaturing step, the samples were returned to 37° C. until the end of the experiment. Once all timepoints had been denatured, the DNA reporter was added to each sample, and fluorescence measurements were begun immediately in the plate reader. (B) Reporter signal starting ˜5 min after the 1_2r gate and 11 samples were mixed, and the 500 nmol/L of the DNA reporter was added (solid lines). The dashed lines represent simulations of this experiment using a kₚ of 0.024 s⁻¹. Results support the strand displacement mechanism because the gate presumably starts in a double stranded form in these experiments.

FIG. 27 shows, according to some embodiments, reactions in the kinetic model of ctRSD circuits. Output templates can also be included in the model to simulate the transcription rate calibration samples.

FIG. 28 shows, according to some embodiments, an uncleaved ctRSD gate (A) produces output >3-fold slower than a cleaved ctRSD gate (B). 25 nmol/L of gate templates were used with 25 nmol/L of 11 or Io, 500 nmol/L of DNA reporter, and 1 U/μL of T7 RNAP. For simulation results (dashed lines), kₚ=0.014 s⁻¹. In (A), two simulation results are shown. The dotted line is from a model in which k_{rsd} was lowered to 1×101 L mol⁻¹ s⁻¹ to simulate a slower forward strand displacement reaction. The dashed line is from a model in which kᵣₑᵥ was increased to 1×10⁶ L mol⁻¹ s⁻¹ to simulate a fast reverse reaction due to a high effective concentration of the output strand. The Io simulation results are the same for both models. These experiments were conducted alongside the experiments presented in FIG. 18 and the 1_2r gate+Io results in (B) are also shown there.

FIG. 29 shows, according to some embodiments, influence of reverse reaction rates on simulation results. The reverse rate must be 10-fold higher to significantly influence the results. (A) Simulations of a one-layer cascade producing O2r. Here, O2r initiates the reverse reaction with the weak b-toehold, which was modeled at 1× as 5 L mol⁻¹ s⁻¹. (B) Simulations of a one-layer cascade producing 01r. Here, O1r initiates the reverse with the a-toehold, which was modeled at 1× as 270 L mol⁻¹ s⁻¹. (C) Simulations of two-, three-, and four-layer cascades producing O2r as the final output.

FIG. 30 shows, according to some embodiments, a leak in ctRSD circuits depends on polymerase concentration and transcriptional load. (A) RNA strand displacement reactions coupled with fluorescence reporting. (B) Normalized reporter signal during transcription of 25 nmol/L of the 1_2r gate template with either 50 nmol/L 11 template (blue line), 50 nmol/L Io template (gray line), or no input template (gray dashed line). From left to right, the concentration of T7 RNAP is increased. Increasing the polymerase concentration increased the rate of output production both with and without input. The transcriptional load in the sample with 50 nmol/L 11 and 25 nmol/L 1_2r gate is higher than the sample with 25 nmol/L 1_2r gate alone. To ensure the transcriptional load between the samples with and without 11 is the same, we tested another sample with the Io template (which produces an input RNA that does not react with the 1_2r gate) instead of the 11 template. Inclusion of the Io template reduces the leak from the 1_2r gate and ensures the same transcriptional load across samples for comparison. 500 nmol/L of DNA reporter was used in all samples.

FIG. 31 shows, according to some embodiments, four different models of ctRSD circuits considered in this study. (A) Schematic of the model without any leak reactions. (B) Simulation results (dashed lines) for the model in (A) compared to experimental results (solid lines). (C) Schematics of the different leak models investigated. Each model includes a different pathway for a leak reaction between the 1_2r gate and the DNA reporter (red reaction lines). In Model 1, the leak is modeled as a 0 base toehold reaction between the cleaved 1_2r gate and the DNA reporter. In Model 2, an additional folding step for the 1_2r gate transcript was introduced into the model. This model assumes the 1_2r gate RNA can react with the DNA reporter complex before the gate has folded. Because such a reaction would use the 6 base b-toehold—as does the designed reaction between the 1_2r strand—the leak was assumed to have the same rate constant as the designed reporting reaction (k_{sd}). In Model 3, it is assumed that a certain percentage of the 1_2r gate transcripts produced are truncated or misfolded. In the case of a truncated product, the b-toehold would be exposed, and thus the truncated product could react with the DNA reporter complex with the same rate constant as the designed reporting reaction.

FIG. 32 shows, according to some embodiments, results for experimentally testing the leak pathways in Models 1, 2, and 3. (A) Experimental set up. The 1_2r gate template or the Io template (negative control) were transcribed with 1 U/μL T7 RNAP for (0.5, 1, or 2) h. After each transcription timepoint, the samples were heated to 65° C. for 30 min to denature T7 RNAP. After the heat denaturing step, the samples were returned to 37° C. until the end of the experiment. Once all timepoints had been denatured, the DNA reporter was added to each sample, and fluorescence measurements were begun immediately in the plate reader. If Model 1 described the leak, the reporter signal should start at 0 and slowly increase. If Model 2 described the leak, the reporter signal should start at 0 and remain there indefinitely because the 1_2r gate is folded and no transcription is occurring. If Model 3 described the leak, the reporter signal should immediately increase above 0. The longer the transcription time before heat denaturing, the higher this initial increase. (B) Normalized reporter signal starting right after 500 nmol/L of the DNA reporter was added to the samples containing the 1_2r gate transcripts. (C) The average normalized reporter signal values from the first 10 min of the data in (B) superimposed over leak of the 1_2r gate transcribed alongside the DNA reporter. (D) Normalized reporter signal from a negative control, in which the Io template was used instead of the 1_2r gate template in the experiment described in (A). This control demonstrates that the assay itself does not influence DNA reporter signal. The results in (B) and (C) support the leak pathway of Model 3 in FIG. 31 and are inconsistent with the pathways of Model 1 and Model 2.

FIG. 33 shows, according to some embodiments, denaturing gel of ctRSD gates with 1, 3, 4, and 5 input domains. All gates self-cleave as designed.

FIG. 34 shows, according to some embodiments, native gels of 3&1_2 AND gates with different sized a′ internal loops between input domains 3 and 1. (A) Schematics of the desired AND gate design. The 3&1_2 gate is designed so that I3 must first react with the gate to expose the toehold for I1 to react and release the output. To accomplish this, we introduced an internal loop in the a:a′-domain between the 3 and 1 duplexes on the AND gate. The gray domains in the internal loops are short linker domains added to reduce strain between the 3 and 1 duplexes. A tradeoff to consider when introducing these internal loops pertains to the following: the a′-loop is the toehold for I1. The longer the a′-loop, the higher the rate at which I1 can react with the AND gate in the absence of I3. We designed AND gates with (3, 4, 5, and 6) base a′-loops to find a design that favored the reaction with 13, while disfavoring the reaction with 11 alone. (B) Schematics of the different AND gate designs tested. (C) Native gel shift assay results for the AND gate variants in (B). In these experiments, a higher molecular weight product should appear both when I3 is present and when I1 and I3 are present. Additionally, 11 by itself should not produce a higher molecular weight product. The AND gate with a 3 base a′-loop does not react with I3 or I1+I3. The AND gate with a 4 base a′-loop reacts ˜50% with I3 and I1+I3. The AND gates with 5 base or 6 base a′-loops fully react with I3 and I1+I3. The larger the a′-loop, the more likely I1 will react with the AND gate in the absence of input. Thus, we selected the 5 base a′-loop design. The samples were run on the gels 2 h after the DNA templates were degraded with DNase 1. The three images were taken from three different gels.

FIG. 35 shows, according to some embodiments, AND gate characterization with the native RNA gel shift assay (A) and the DNA reporter assay (B). The 5&4_2r experiment was conducted using the same gate, input(s), and T7 RNAP concentrations as the 3&1_2r circuit element (TABLE 4). For the gel electrophoresis results, gate and input templates were at 25 nmol/L and 50 nmol/L (2×), respectively. Electrophoresis was conducted 1 h after DNase I addition. The gate′ strand is from the 3&1_2r gate. The first seven lanes of the gel are also presented in FIG. 4E. The DNA reporter 3&1_2r results are also presented in FIG. 4F.

FIG. 36 shows, according to some embodiments, steric hindrance between the leak products and ctRSD gates reduces the leak observed in experiments. (A) Nothing upstream of the 5′ toehold of the DNA reporter sterically hinders a bulky leak product. Thus, the leak product likely reacts with the DNA reporter at a similar rate as a single-stranded output. (B and C) Single-input ctRSD gates (B) and ctRSD AND gates (C) have duplexes upstream of their 5′ toehold, which could sterically hinder a reaction with a bulky leak product. Note the leak products shown here are hypothetical but are representative of the true leak products, which are likely bulkier than their ssRNA output strands. (D) Schematics of the single-stranded 5_1 output strand and the 5_1 leak hairpin product used in the experiments in (E) and (F). The 5_1 HDV cau transcript contained the output strand of the 5_1 gate, followed by the HDV ribozyme and a 22 base sequence composed of only C, A, U bases. Upon transcription, this transcript cleaves to produce the output from the 5_1 gate. The 5_1 leak hairpin transcript was truncated such that the a-toehold of the 5_1 gate output was exposed. Further, an inactive ribozyme variant (xRz) was used to ensure the transcript remained in a hairpin structure. (E) Experimental (solid) and simulated (dashed) reporter signal during co-transcription of the 1_2r gate and the RNA products in (D). (F) Experimental (solid) and simulated (dashed) reporter signal during co-transcription of the 3&1_2r gate and I3 and the RNA products in (D). The color of the experimental data corresponds to the color of the transcript names in (D). [1_2r gate template]=[3&1_2r gate template]=[I3 template]=25 nmol/L. [5_1 HDV cau template]=[5_1 leak hairpin template]=10 nmol/L. Io was added to bring the total template concentration up to 75 nmol/L in each sample. [T7 RNAP]=1 U/μL. For the simulations, a kₚ of 0.01 s⁻¹ was used, and the RNA strand displacement rate constant between the 5_1 leak product and the gates was varied to recapitulate the experimental kinetics. The reaction rate constant with the leak reaction was approximately two orders of magnitude lower than the rate constant for the reaction with the single-stranded 5_1 output. The rate constants are given inside the plots.

FIG. 37 shows, according to some embodiments, lowering k_{rsd} for I4 4-fold better recapitulates experimental results. (A) The single-layer cascade of 14 to 4_2r from FIG. 8A with simulation results using k_{rsd}=1×10³ L mol⁻¹ s⁻¹ (left) or 2.5×10² L mol⁻¹ s⁻¹ (right). (B) A two-layer cascade of 14 to 4_1 to 1_2r (purple) or 15 to 5_1 to 1_2r (teal) with simulation results using k_{rsd}=1×10³ L mol⁻¹ s⁻¹ (left) or 2.5×10² L mol⁻¹ s⁻¹ (right). (C) The four-layer cascade from FIG. 10B with simulation results using k_{rsd}=1×10³ L mol⁻¹ s⁻¹ (left) or 2.5×10² L mol⁻¹ s⁻¹ (right). (D) The logic cascade containing the 4_1 gate from FIG. 5E with simulation results using k_{rsd}=1×10³ L mol⁻¹ s⁻¹ (left) or 2.5×10² L mol⁻¹ s⁻¹ (right). All other rate constants are presented in TABLE 3.

FIG. 38 shows, according to some embodiments, denaturing gel of 1_2r gates with (6, 8, 10, and 12) base a′-toeholds. All gates self-cleave as designed.

FIG. 39 shows, according to some embodiments, normalized reporter signal during co-transcription of Io and 1_2r gate variants with differing a′-toehold lengths. Increasing toehold length did not increase the observed amount of downstream reporter leak. [1_2r gate templates]=5 nmol/L, [Io template]=40 nmol/L, [DNA reporter]=150 nmol/L, and [T7 RNAP]=1 U/μL.

FIG. 40 shows, according to some embodiments, ctRSD circuit kinetics depend on toehold length and the length of a single-stranded spacer after the self-cleaving ribozyme. (A) Schematic of I1 bound to a 1_2r gate with a single-stranded spacer. (B) Grid representing the combination of I1 a-toehold lengths and 1_2r gate a′-toehold lengths tested in experiments. Box shading represents the length of the spacer for each combination. The numbers inside the boxes are the k_{rsd} reaction rate constants, in units of L mol⁻¹ s⁻¹, that recapitulated the experimental data for each combination. Under the experimental conditions, estimates of reaction rate constants >1×10⁵ L mol⁻¹ s⁻¹ could not be distinguished (see panel C) and are reported as ≥1×10⁵ L mol⁻¹ s⁻¹. (C) Simulations of co-transcription of I1 and the 1_2r gate with k_{rsd} rate constants ranging six orders of magnitude. These simulations show rate constants >1×10⁵ L mol⁻¹ s⁻¹ result in similar kinetics and are indistinguishable in this assay. (D) Normalized reporter signal for the 11 and 1_2r gate combinations in (B). Here, each plot shows a single 11 variant with different 1_2r gate variants to explore the effect of spacer length on reaction kinetics. Each plot represents a single row from the grid in (B). (E) Normalized reporter signal for the 11 and 1_2r gate combinations in (B). Here, each plot shows 11 and 1_2r gate variant combinations with constant spacer lengths to explore the effect of toehold length on reaction kinetics. Each plot represents a left to right diagonal of equal shading from the grid in (B). [1_2r gate templates]=5 nmol/L, [I1 templates]=5 nmol/L, [DNA reporter]=150 nmol/L, and [T7 RNAP]=1 U/μL. Io was added to bring the total template concentration up to 45 nmol/L in each sample.

For the simulations, kₚ of 0.019 s⁻¹ was used, and the k_{rsd} reaction rate constants were calibrated to the experimental kinetics. The simulation results in (D and E) used the k_{rsd} values tabulated in (B). Schematics with sequences of the input and gate variants are presented in FIG. 16.

FIG. 41 shows, according to some embodiments, total template concentration and T7 RNAP concentration influence transcriptional load. (A) Schematic of the loading and reporting reactions. The Io template was added at different concentrations to change the total transcriptional load, and the O2r strand was constitutively produced from the 1_2r HDV cau template. The 1_2r HDV cau template was chosen to match the total number of bases in the 1_2r gate template. (B) Experimental (solid lines) and simulated (dashed lines) reporter signal during transcription of the 1_2r HDV cau RNA with different Io template concentrations and T7 RNAP concentrations. The kₚ values used in the simulations are tabulated for the different Io template concentrations and T7 RNAP concentrations. Increasing the Io template concentration (transcriptional load) decreases the effective kₚ value and increasing the T7 RNAP concentration increases the effective kₚ value. 25 nmol/L of the 1_2r HDV cau template and 500 nmol/L of the DNA reporter were used in each experiment.

FIG. 42 shows, according to some embodiments, representative examples showing calibration of the transcription rate constant for experiments with different transcriptional loads and/or T7 RNAP concentrations. The 1_2r HDV cau template, which constitutively produces the O2r strand, was used as a reference sample for each experiment. The Io template was added to bring the total template concentration in the reference sample to the total concentration used in the experimental samples. The reference sample was then used to calibrate the first order transcription rate constant, kₚ, for simulation of the experimental samples.

FIG. 43 shows, according to some embodiments, variation across independently prepared technical replicates for ctRSD circuit reactions using the 1_2r gate (A) or the 5&4_1+3&1_2r gates (B). Left plots show data for three independently prepared replicates of the same ctRSD circuit reaction. The replicates were prepared from the same stock solutions on the same day. The right plots show the mean of the three replicates; error bars represent one standard deviation. The standard deviation was <1.5% from the mean value at each time point for (A) and <5% from the mean value at each time point for (B). In (A), reactions included 25 nmol/L of the 1_2r gate template, 12.5 nmol/L of the 11 template, and Io template to bring the total template to 50 nmol/L in both reactions. In (B), reactions included 25 nmol/L of the 5&4_1, 25 nmol/L of the 3&1_2r gate, and 25 nmol/L of the input templates. For the samples containing I3 only, 50 nmol/L of the Io template was added to bring the total template concentration to 75 nmol/L. In all experiments, 500 nmol/L of the DNA reporter and 2 U/μL of T7 RNAP was used.

FIG. 44 shows, according to some embodiments, variation across experiments performed on different days for the same set of conditions. Left plots show data for three independent replicates of the same ctRSD circuit reaction. Reactions included 25 nmol/L of the 1_2r gate template, 500 nmol/L of the DNA reporter, 1 U/μL of T7 RNAP, and 50 nmol/L of the 11 or Io templates. The three individual replicates were prepared independently and tested on separate days, with the second and third replicates conducted 5 and 19 days after the first replicate, respectively. Dark colored lines represent the oldest replicate; data also presented in FIG. 30B, middle panel. Medium colored lines represent the second oldest replicate;. Light colored lines represent the newest replicate; data also presented in FIG. 28B. The right plot shows the mean of the three replicates; error bars represent one standard deviation. The standard deviation is <3% from the mean value at each time point.

### DETAILED DESCRIPTION

A detailed description of one or more embodiments is presented herein and the accompanying parts of the specification by way of exemplification and not limitation.

Engineered molecular circuits process information in biological systems and can address emerging human health and biomanufacturing needs. A scalable co-transcriptional RNA strand displacement (ctRSD) circuit is rationally programmed via base pairing interactions. Conventional DNA-based strand displacement circuits can be computationally powerful molecular circuits but are limited in biological systems due to difficulty in genetically encoding components. The ctRSD overcomes this limitation of such conventional technology by isothermally producing circuit components via transcription. The programmability of ctRSD in vitro occurs by designing logic and amplification elements and multi-layer signaling cascades. Further, kinetics of ctRSD are predicted by a model of coupled transcription and strand displacement. The ctRSD provides rational design of molecular circuits that operate in biological systems, including living cells.

It is contemplated that co-transcriptional RNA strand displacement (ctRSD) circuits are scalable and programmable. In ctRSD, circuit components isothermally self-assemble and execute programmed computations in a single transcription reaction. This is achieved through an HDV self-cleaving ribozyme to isothermally prepare kinetically trapped RNA strand displacement intermediates via transcription, and a set of nucleic acid sequence design rules that allow mutiple RNA strand displacement sequences with similar performance to be readily created. The ctRSD overcomes limitations of conventional DNA-based strand displacement such as degradation in biological environments and single-use operation. Moreover, ctRSD provides nucleic acid strand displacement circuits that are genetically encoded into living cells for cellular engineering applications.

Conventional DNA-based strand displacement circuits are a molecular computing paradigm. However, conventional DNA circuits are susceptible to degradation in biological systems. Further, conventional DNA-based circuits are only single-use, wherein they can only execute one computation unless their components are replenished via external perturbation. Finally, there is currently no mechanism to produce these state-of-the-art circuits in the same sample where they operate.

Advantageously and unexpectedly, co-transcriptional RNA strand displacement circuits provide powerful computing features of DNA-based circuits and can be genetically encoded to overcome limitations of conventional DNA-based circuits in biological systems. Co-transcriptional RNA strand displacement circuits can be encoded into living cells for the same programmability and functionality of DNA-based circuits for cellular engineering applications. Co-transcriptional RNA strand displacement circuits provide real-time cell state monitoring through recognition of differential RNA expression patterns. Co-transcriptional RNA strand displacement circuits can provide real-time monitoring of cell-state to improve biomanufacturing processes or for real-time detection of cellular disease states. Nucleic acid pattern recognition has occurred with DNA-based circuits in vitro but has never been demonstrated in living cells, something which co-transcriptional RNA strand displacement circuits can provide for engineering cellular sensing and response.

Co-transcriptional RNA strand displacement circuits can be applied in in vitro environments. Co-transcriptional RNA strand displacement circuits can be used in an in vitro transcription-based biosensor for detecting water contaminants, wherien such biosensors provide more sophisticated computations to be executed than conventional technology.

Although DNA-based strand displacement components can expand computational capabilities, such biosensors are often freeze-dried for long-term storage and transport, and a limitation of using DNA-based components in these sensors is that the DNA strand displacement components result in much shorter shelf-lives when freeze dried compared to longer transcription templates or plasmids. For example, the DNA-based components showed significant decrease in performance only one week after freeze drying. In contrast, long linear DNA templates have been shown to be stable for over a month and DNA plasmids containing transcription templates have been shown to be stable for 2 years after freeze drying. Thus, encoding co-transcriptional RNA strand displacement components in long linear templates or in plasmids offers the same functionality as existing DNA circuits but with improved stability in freeze dried samples.

Certain in vitro sensors for detecting viral infections and other diseases operate by detecting specific RNA sequences that then trigger the production of a fluorescent output. Co-transcriptional RNA strand displacement circuits can be an upstream information processing layer in such diagnostics.

The use of co-transcriptional RNA strand displacement circuits in these diagnostics could enable more complex computations, such as mathematical operations or neural network pattern recognition. These capabilities could enable more robust and reliable diagnostics by integrating more input information before making a diagnosis.

Co-transcriptional RNA strand displacement provides sophisticated DNA-based diagnostics to be robustly operated in biological systems. Certain conventional DNA-based molecular neural networks recognize differential gene expression levels associated with cancers, but that circuit has been operated in a pure in vitro setting. Using co-transcriptional RNA strand displacement provides this sophisticated diagnostic circuit to robustly operate in blood or fecal samples where DNA-based circuits would be limited by degradation.

The ctRSD provides a predictive engineering of biology and programmable cellular engineering. Beneficially, modular RNA gates are isothermally produced in a kinetically trapped form in the same reaction vessel. This has not been achieved in conventional DNA-based systems.

In an embodiment, a ctRSD gate (200) for performing co-transcriptional encoding comprises: an output strand (201) comprising: an input branch migration domain (206); an output branch migration domain (204) sequentially connected to the input branch migration domain (206); and an output toehold domain (205) sequentially interposed between the input branch migration domain (206) and the output branch migration domain (204); and a gate prime strand (202) electrostatically associated with the output strand (201) and comprising; a self-cleaving ribozyme (209); an output toehold sequester domain (213) sequentially connected to the self-cleaving ribozyme (209); a substrate domain (211) sequentially interposed between the self-cleaving ribozyme (209) and the output toehold sequester domain (213), such that a portion of the substrate domain (211) is sequentially complementary to a portion of the input branch migration domain (206) that results in the gate prime strand (202) being electrostatically associated with the output strand (201); and an input toehold domain (210) sequentially interposed between the self-cleaving ribozyme (209) and the substrate domain (211),wherein the output strand (201) and the gate prime strand (202) indepedently consist essentially of RNA.

In an embodiment, the output strand (201) further comprises: a hairpin-forming sequence (203) sequentially connected to the output branch migration domain (204) such that output branch migration domain (204) is sequentially interposed between the hairpin-forming sequence (203) and the output toehold domain (205).

In an embodiment, the output strand (201) further comprises: an output wobble domain (207) sequentially connected to the input branch migration domain (206) such that the output wobble domain (207) is sequentially interposed between a first portion of the input branch migration domain (206) and a second portion of the input branch migration domain (206).

In an embodiment, the output strand (201) further comprises: a linker sequence (208) sequentially connected to the input branch migration domain (206) such that input branch migration domain (206) is sequentially interposed between the linker sequence (208) and the linker sequence (208).

In an embodiment, the gate prime strand (202) further comprises: a transcription termination sequence (214) sequentially connected to the output toehold sequester domain (213) such that output toehold sequester domain (213) is sequentially interposed between the transcription termination sequence (214) and the substrate domain (211).

In an embodiment, the gate prime strand (202) further comprises: a gate prime wobble domain (212) sequentially connected to the substrate domain (211) such that the gate prime wobble domain (212) is sequentially interposed between a first portion of the substrate domain (211) and a second portion of the substrate domain (211).

In an embodiment, the output strand (201) produces a strand displacement product (216) in response to contact with an input template strand (215).

In an embodiment, the output strand (201) further comprises a second input branch migration domain (206).2 sequentially connected to the input branch migration domain (206).

In an embodiment, the gate prime strand (202) further comprises a second substrate domain (211).2 sequentially connected to the substrate domain (211).

In an embodiment, a process for producing a strand displacement product (216) comprises: providing a ctRSD gate (200); contacting the ctRSD gate (200) with a input template strand (215); and producing the strand displacement product (216) from the ctRSD gate (200) in response to contacting the ctRSD gate (200) with the input template strand (215).

In an embodiment of the process for producing the strand displacement product (216), the ctRSD gate (200) comprises: an output strand (201) comprising: an input branch migration domain (206); an output branch migration domain (204) sequentially connected to the input branch migration domain (206); and an output toehold domain (205) sequentially interposed between the input branch migration domain (206) and the output branch migration domain (204); and a gate prime strand (202) electrostatically associated with the output strand (201) and comprising; a self-cleaving ribozyme (209); an output toehold sequester domain (213) sequentially connected to the self-cleaving ribozyme (209); a substrate domain (211) sequentially interposed between the self-cleaving ribozyme (209) and the output toehold sequester domain (213), such that a portion of the substrate domain (211) is sequentially complementary to a portion of the input branch migration domain (206) that results in the gate prime strand (202) being electrostatically associated with the output strand (201); and an input toehold domain (210) sequentially interposed between the self-cleaving ribozyme (209) and the substrate domain (211), wherein the output strand (201) and the gate prime strand (202) indepedently consist essentially of RNA.

In an embodiment of the process for producing the strand displacement product (216), the output strand (201) further comprises: a hairpin-forming sequence (203) sequentially connected to the output branch migration domain (204) such that output branch migration domain (204) is sequentially interposed between the hairpin-forming sequence (203) and the output toehold domain (205).

In an embodiment of the process for producing the strand displacement product (216), the output strand (201) further comprises: an output wobble domain (207) sequentially connected to the input branch migration domain (206) such that the output wobble domain (207) is sequentially interposed between a first portion of the input branch migration domain (206) and a second portion of the input branch migration domain (206).

In an embodiment of the process for producing the strand displacement product (216), the output strand (201) further comprises: a linker sequence (208) sequentially connected to the input branch migration domain (206) such that input branch migration domain (206) is sequentially interposed between the linker sequence (208) and the linker sequence (208).

In an embodiment of the process for producing the strand displacement product (216), the gate prime strand (202) further comprises: a transcription termination sequence (214) sequentially connected to the output toehold sequester domain (213) such that output toehold sequester domain (213) is sequentially interposed between the transcription termination sequence (214) and the substrate domain (211).

In an embodiment of the process for producing the strand displacement product (216), the gate prime strand (202) further comprises: a gate prime wobble domain (212) sequentially connected to the substrate domain (211) such that the gate prime wobble domain (212) is sequentially interposed between a first portion of the substrate domain (211) and a second portion of the substrate domain (211).

The articles and processes herein are illustrated further by the following Example, which is non-limiting.

### Example

Engineered molecular circuits that process information in biological systems could address emerging human health and biomanufacturing needs. However, such circuits can be difficult to rationally design and scale. DNA-based strand displacement reactions have demonstrated the largest and most computationally powerful molecular circuits to date but are limited in biological systems due to the difficulty in genetically encoding components. Here, this Example describes scalable co-transcriptionally encoded RNA strand displacement (ctRSD) circuits that are rationally programmed via base pairing interactions. ctRSD circuits address the limitations of DNA-based strand displacement circuits by isothermally producing circuit components via transcription. We demonstrate circuit programmability in vitro by implementing logic and amplification elements, and multi-layer cascades. Further, we show circuit kinetics are accurately predicted by a simple model of coupled transcription and strand displacement, enabling model-driven design. It is contemplated that ctRSD circuits provide rational design of molecular circuits that operate in biological systems, including living cells.

This Example describes self-assembling RNA circuits for synthetic biology that execute programmable logic, amplification, and cascades.

A goal of synthetic biology is developing programmable molecular circuits that can be rationally engineered to process information in biological systems. Such circuits have the potential to address emerging challenges in human health and disease, agriculture, and biomanufacturing. To meet these diverse needs, molecular circuits must be scalable, modular, and rationally programmable to execute operations like logic, signal amplification, and multi-layer cascades. Further, circuits capable of a wide range of computations beyond Boolean logic, such as molecular pattern recognition, could greatly expand existing capabilities. A key challenge to developing such circuits is identifying molecular components that not only meet the above criteria, but also behave predictably to enable model-driven design.

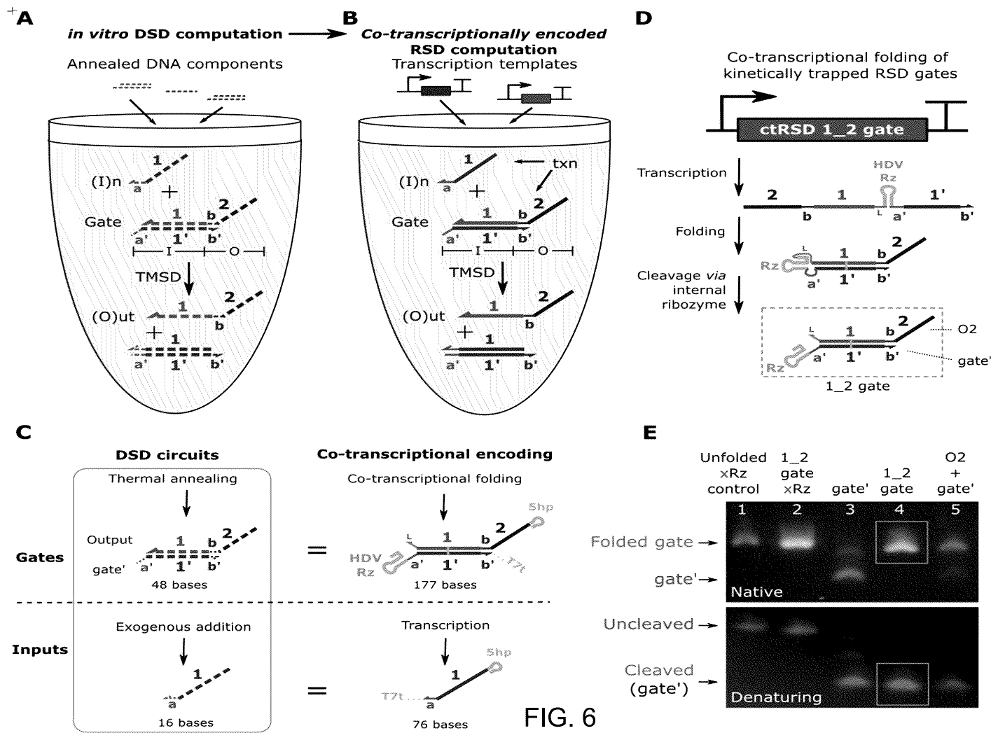

The predictable and programmable Watson-Crick base pairing interactions of nucleic acids has led to their adoption as versatile components for molecular circuit programming. In particular, in vitro circuits based on toehold-mediated strand displacement (TMSD) reactions have demonstrated sophisticated digital computations and mathematical operations, molecular pattern recognition, signal cascades and amplifiers, and complex dynamics. In TMSD reactions, a single-stranded input binds to a double-stranded nucleic acid gate via a single-stranded toehold domain and displaces an output strand with a new exposed toehold that can facilitate further TMSD reactions (FIG. 6A). Interactions between inputs and gates are programmed through sequence complementarity, and the combinatorial nucleic acid sequence space allows TMSD reaction networks to be scaled up to >100 components. Additionally, reaction kinetics can be tuned over six orders of magnitude by simply changing the length of the toehold. These properties have enabled predictive models of TMSD circuit behavior that allow circuit design abstraction.

Because TMSD circuits are composed of nucleic acids, they have great potential for integration with biological systems. However, these circuits have primarily been implemented in vitro using DNA components that are not easily genetically encoded. This restricts their applications in synthetic biology, particularly in vivo. A challenge to operating TMSD circuits in biological systems is developing a method to isothermally prepare all circuit components in a single reaction. Typically, TMSD components are thermally annealed separately to prevent spurious reactions between gates and then mixed to make a circuit. Thus, these circuits currently cannot be continuously produced in the same place they are operated. Although TMSD circuits can be prepared and then added to biological samples or transfected into cells at fixed concentrations, these implementations are only single use and circuit lifetime is limited by component degradation. A method to continuously produce TMSD circuits in situ can greatly expand their capabilities. Genetically encoded RNA-based circuits that utilize strand displacement have been developed and other transcription-based circuits have achieved some of the capabilities of TMSD circuits. However, these systems have yet to demonstrate the predictive design and scale up seen in state-of-the-art DNA-based circuits.

This Examples describes scalable and programmable co-transcriptionally encoded RNA strand displacement (ctRSD) circuits. In ctRSD circuits, components isothermally self-assemble during transcription and execute programmed computations in the same reaction. We validate ctRSD circuit performance in vitro by building circuits that execute logic, signal amplification, and multi-layer cascades. We demonstrate the scalability and modularity of the ctRSD by successfully implementing 13 ctRSD gates in 8 different circuit topologies. We find ctRSD kinetics are well predicted by a simple model of coupled transcription and strand displacement that assumes uniform kinetic behavior across gates, facilitating predictive circuit engineering. Further, ctRSD circuits are designed so that state-of-the-art DNA-based circuits capable of neural network computations and pattern recognition could be directly adopted. ctRSD should enable the power of TMSD circuits to be realized in biological systems for smart diagnostics or sensors. Ultimately, ctRSD circuits could be genetically encoded and continuously operated inside living cells.

Design of Co-Transcriptionally Encoded RNA Strand Displacement (ctRSD) Circuit Components

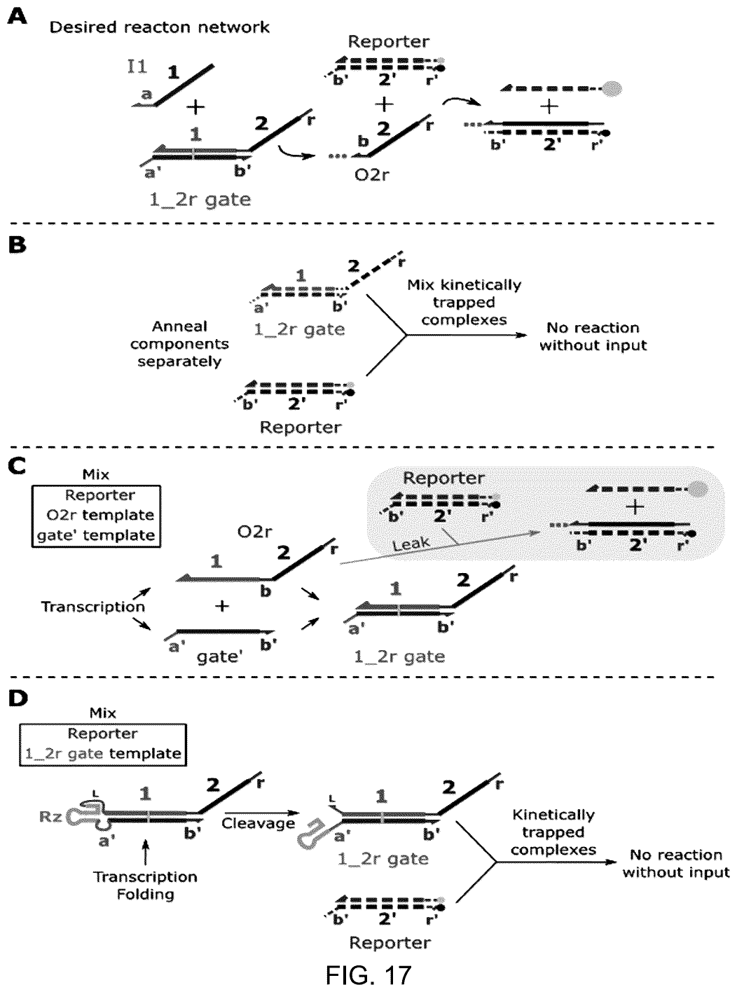

To develop ctRSD circuits, a system includes modular and programmable strand displacement circuit components that can be isothermally produced via transcription. In TMSD circuits, modularity is achieved by designing toehold exchange gates that allow any input sequence to be converted into any output sequence through a gate. For example, in FIG. 6A, the input domain is composed of the a′-toehold and the 1:1′-domain duplex, both of which are complementary to the input, 11. The output domain is composed of the sequestered b-toehold and the 2-domain overhang, neither of which share complementarity with 11. Thus, input and output domain sequences are completely independent. We adopted an analogous modular gate design for ctRSD (FIG. 6B). In these toehold exchange gates, the b-toehold of the output is sequestered in a duplex, kinetically precluding a reaction downstream unless the gate input is present. In DNA-based circuits, the toehold exchange gates are thermally annealed in separate test tubes to kinetically trap the outputs before circuit components are mixed. In ctRSD circuits, the RNA toehold exchange gates must isothermally assemble into kinetically trapped intermediates in a single pot during transcription. Simply transcribing the two gate strands separately and allowing them to hybridize to form a gate is not a viable option as the output strand of the gate can also react with downstream gates, introducing significant leak (FIG. 17).

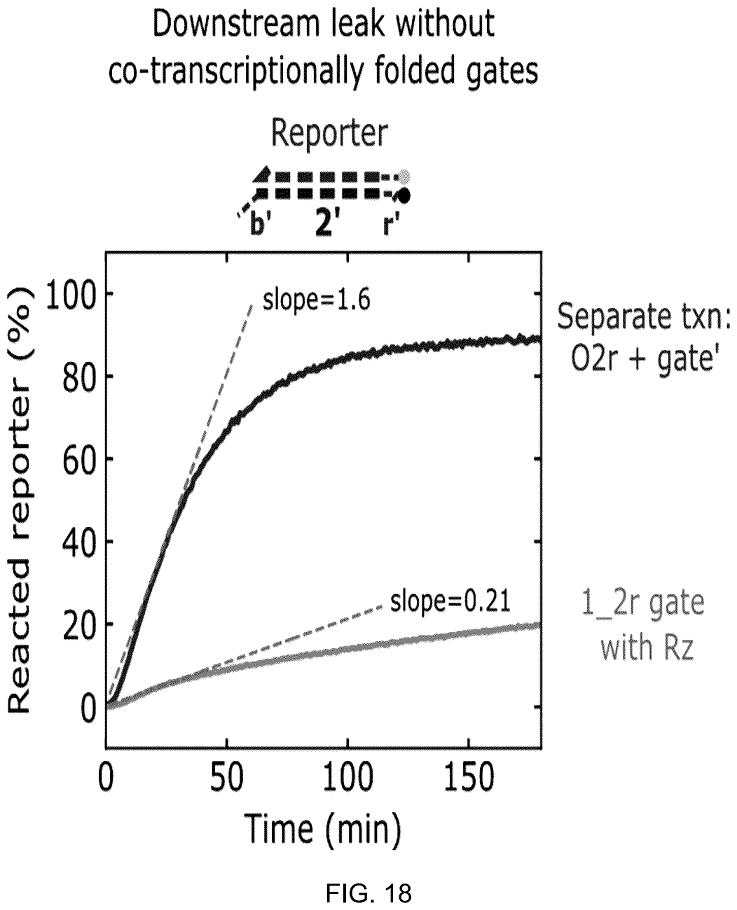

To transcriptionally encode kinetically trapped RNA toehold exchange gates, we inserted a self-cleaving RNA ribozyme motif between the two strands of the gate (FIG. 6C). This motif allows us to encode RNA gates as single transcripts that fold into hairpins and then cleave to yield reactive gates (FIG. 6D). Co-transcriptional folding is at least one order of magnitude faster than transcription, so the RNA gates should fold before they have time to react downstream. The self-cleaving ribozyme also ensures 1:1 stoichiometry between the gate strands, further reducing the potential for leaks. Inclusion of the ribozyme motif is critical, as the co-transcriptionally folded RNA gate exhibited >7-fold lower downstream leak rate than transcribing the two strands of the RNA gate separately (FIG. 18). A 5′ hairpin motif and a 3′ hairpin terminator for T7 RNAP were also appended to gates and inputs (FIG. 6C). The 5′ hairpin contains the T7 RNAP consensus initiation sequence to facilitate efficient and uniform transcription across components. Additionally, the 5′ hairpin ensures that short abortive transcripts produced during transcription initiation will not have sequence overlap with other circuit elements. The terminator hairpin reduces unwanted products associated with runoff transcription and enables incorporation into plasmids.

FIG. 6C shows the final selection for our ctRSD gate design, however, there are alternative implementations that would embody the same general features. To optimize gate performance, we analyzed four considerations when selecting the final design: 1) directionality of the single-stranded toehold, 2) domain sequence identity, 3) domain transcription order, and 4) self-cleaving ribozyme choice. We designed the ctRSD gates with 5′ toeholds because a 5′ toehold on an RNA gate allows the invading strand to participate in co-axial base stacking, increasing the binding strength compared to a 3′ toehold. We restricted the gate output sequences to cytosine (C), adenine (A), or uracil (U) bases. This sequence constraint reduces unwanted secondary structure or dimerization of single-stranded components. A G-U wobble pair was also introduced in the middle of the hybridized portion of the gate to reduce DNA template synthesis errors and to drive the forward ctRSD reaction with inputs that convert the G-U wobble to a G-C pair. The 5′ end of the output strand of the gate was selected as the starting point for transcription so that the first sequence produced would only possess C, A, and U bases, preventing co-transcriptional folding into undesired secondary structure. This transcription order ensures that the G, A, and U restricted sequence of the strand that hybridizes to the output strand (i.e. the gate′ strand) is transcribed after its complementary sequence to promote folding of the RNA gate stem over alternative structures with G-U wobbles. For the self-cleaving ribozyme, we selected a variant of the hepatitis delta virus (HDV) ribozyme This ribozyme has no upstream or downstream sequence constraints, has a very stable fold, and has been reported to cleave itself with a rate constant of nearly 1 s⁻¹ in vivo.

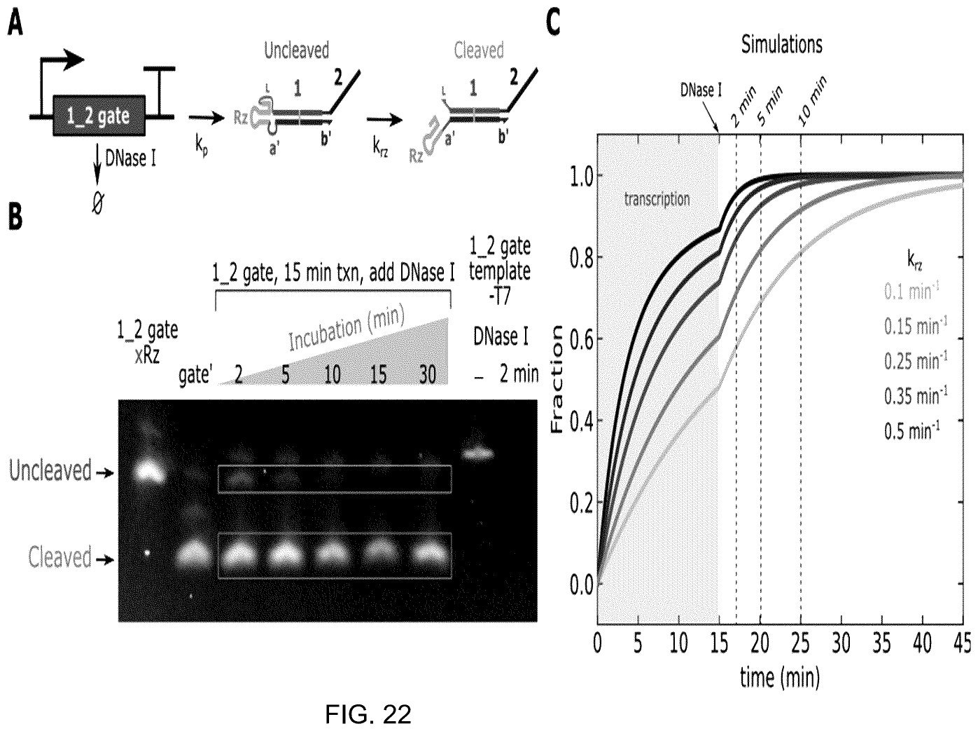

We used native and denaturing agarose gel electrophoresis to confirm the ctRSD gate fold and cleave as designed. On a native gel, the ctRSD gate (lane 4, FIG. 6E) was the same size as a control sample in which the two strands of the gate were transcribed from separate templates (lane 5, FIG. 6E), indicating full length gate production and folding. On a denaturing gel, the primary product from the ctRSD gate (lane 4, FIG. 6E) migrated faster than the uncleaved control transcript (lane 2, FIG. 6E) and was the same size as the gate′ strand alone (lane 3, FIG. 6E), indicating ribozyme cleavage. Importantly, the cleavage reaction is efficient and fast, we observed >90% cleavage in less than 15 min with an estimated cleavage rate constant of 0.25 min⁻¹ (FIG. 22).

Experimental Characterization and Modeling of ctRSD Circuits

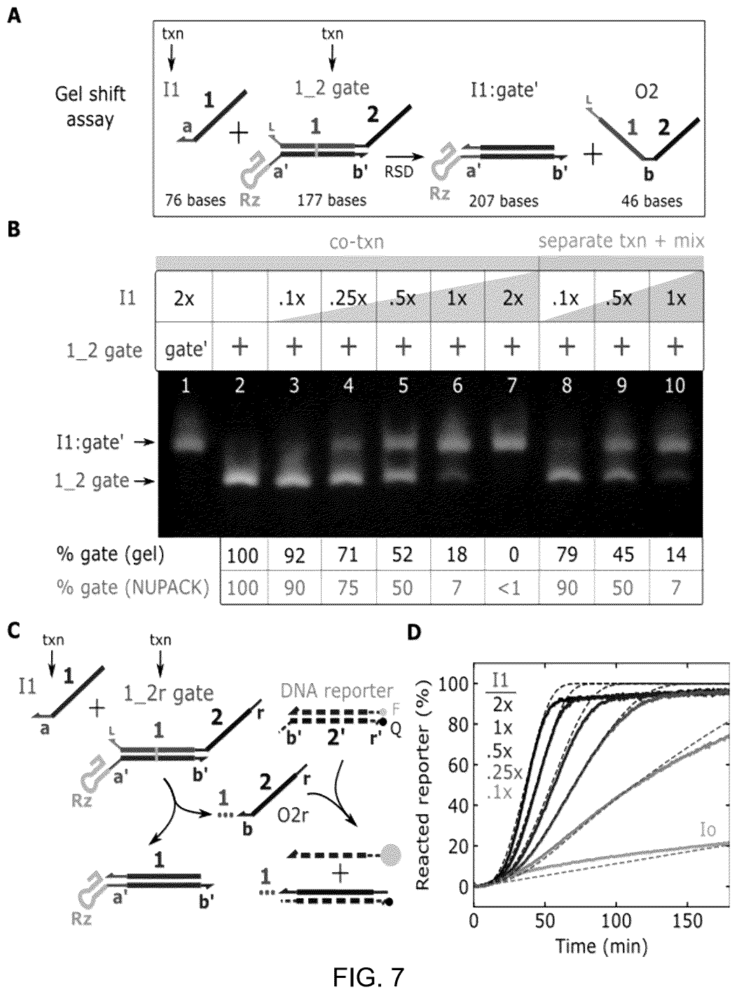

We next sought to characterize the reaction in which a ctRSD gate and its corresponding input are co-transcribed and react via strand displacement to release an output strand (FIG. 6B). The 11:gate′ product of the strand displacement reaction is a higher molecular weight than the unreacted gate, so we first analyzed the reaction with native gel electrophoresis (FIG. 7A). Increasing concentrations of 11 template increased the percentage of I1:gate′ product on the gel, with a 2:1 mixture of the I1 and 1_2 gate templates yielding 100% product (lane 3 to lane 7, FIG. 7B). Assuming the transcription rates of 11 and the 1_2 gate are approximately equal, the fraction of I1:gate′ produced with increasing 11 template concentration provides information about the thermodynamics of the reaction. We found the unreacted 1_2 gate percentages across input concentrations in experiments were within ˜12% of the thermodynamic predictions from NUPACK 3.2.2 (FIG. 7B).

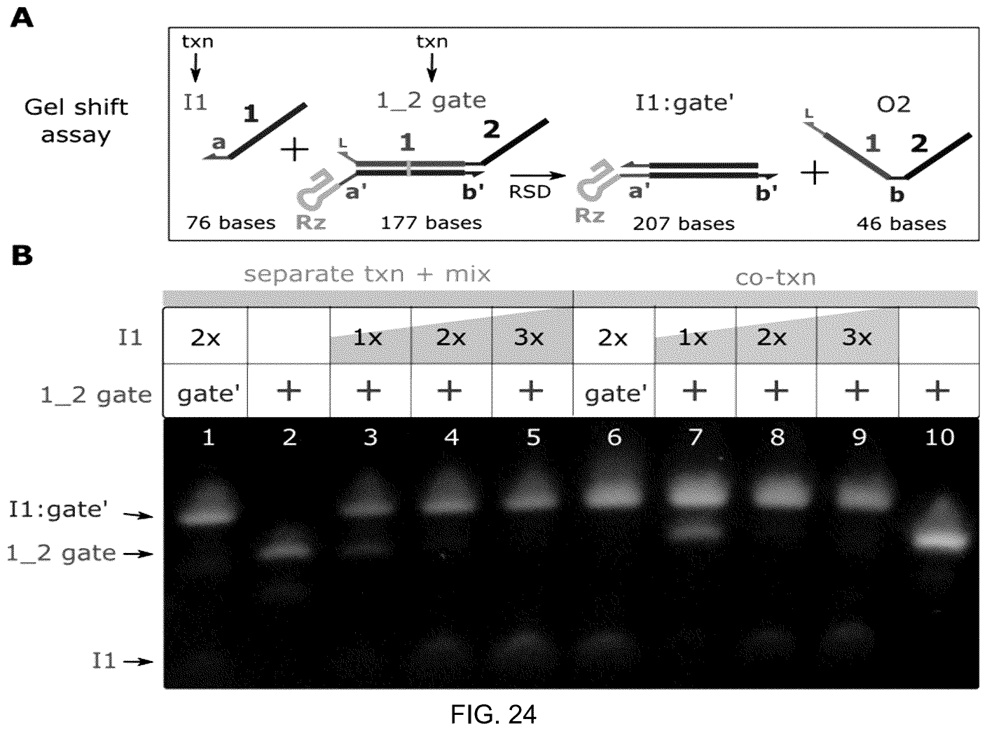

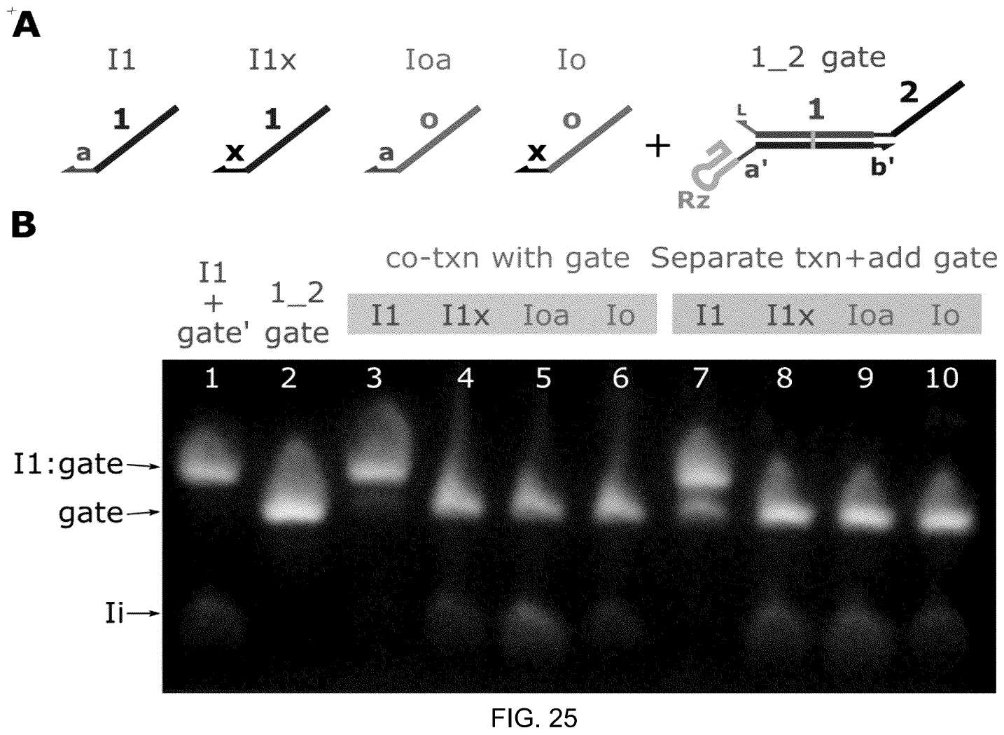

The results in lane 3 to lane 7 in FIG. 7B were obtained from simultaneous transcription of I1 and the 1_2 gate, so the observed reaction between the two transcripts could result from I1 binding to the 1_2 gate prior to folding, rather than strand displacement. To rule out this potential reaction pathway, we transcribed the I1 and the 1_2 gate RNAs separately and then mixed them together after degradation of the DNA templates. Separate transcription followed by mixing yielded similar results to co-transcription (lane 8 to lane 10, FIG. 7B and FIG. 24), wherein I1 and the 1_2 gate react via the designed strand displacement mechanism. We further confirmed experimentally that the correct a-toehold sequence and 1-domain branch migration sequence were required for strand displacement (FIG. 25).

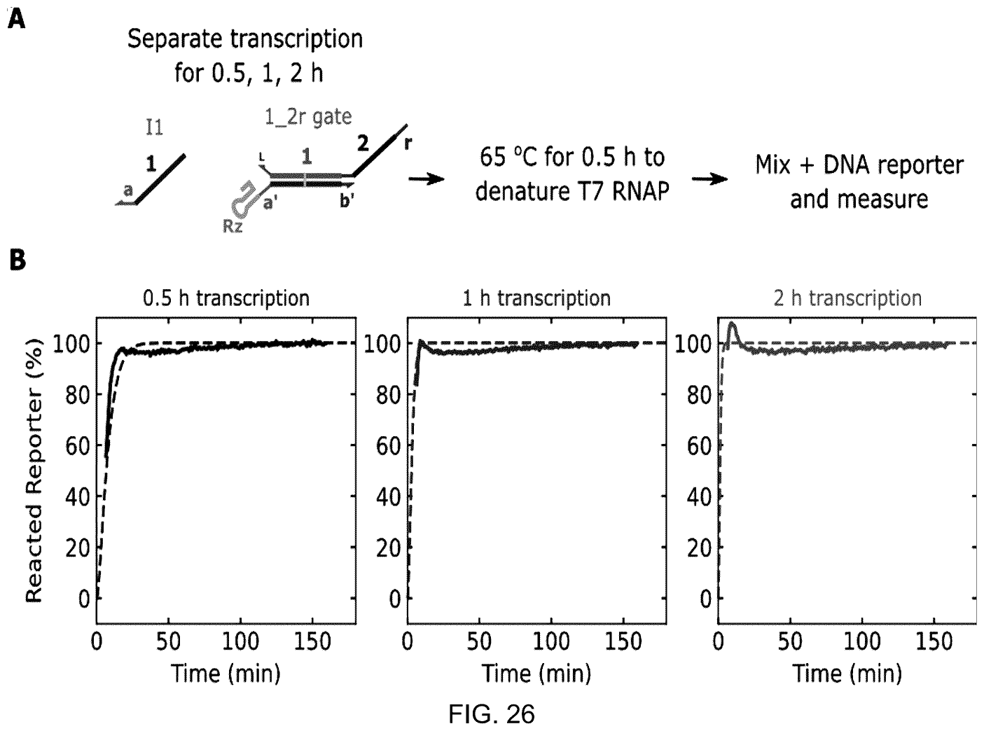

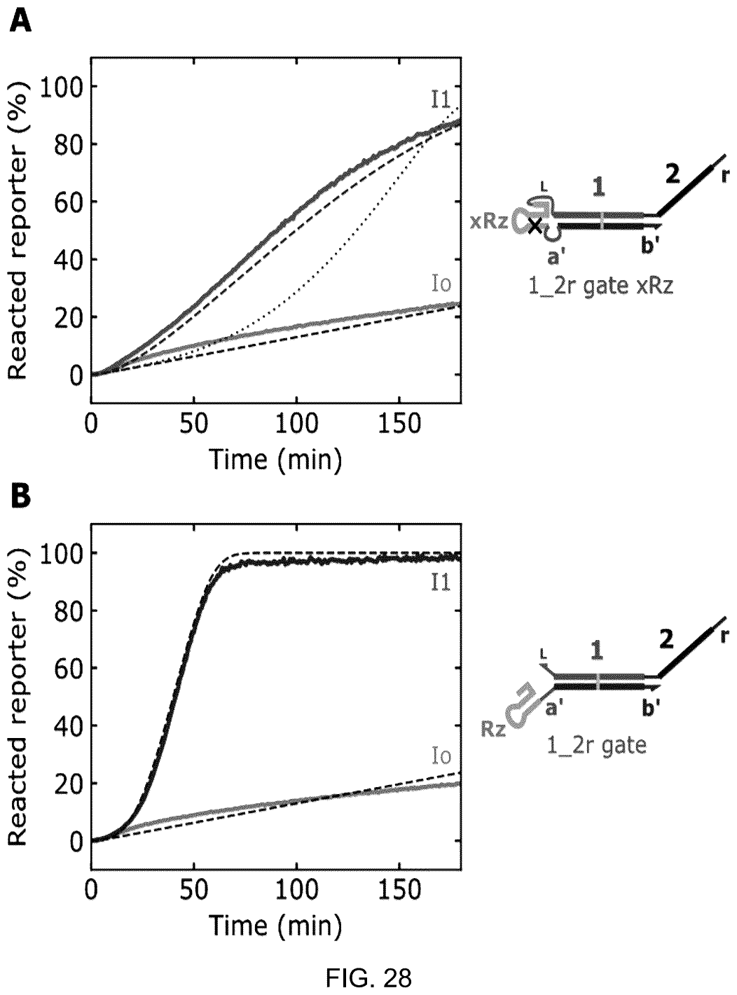

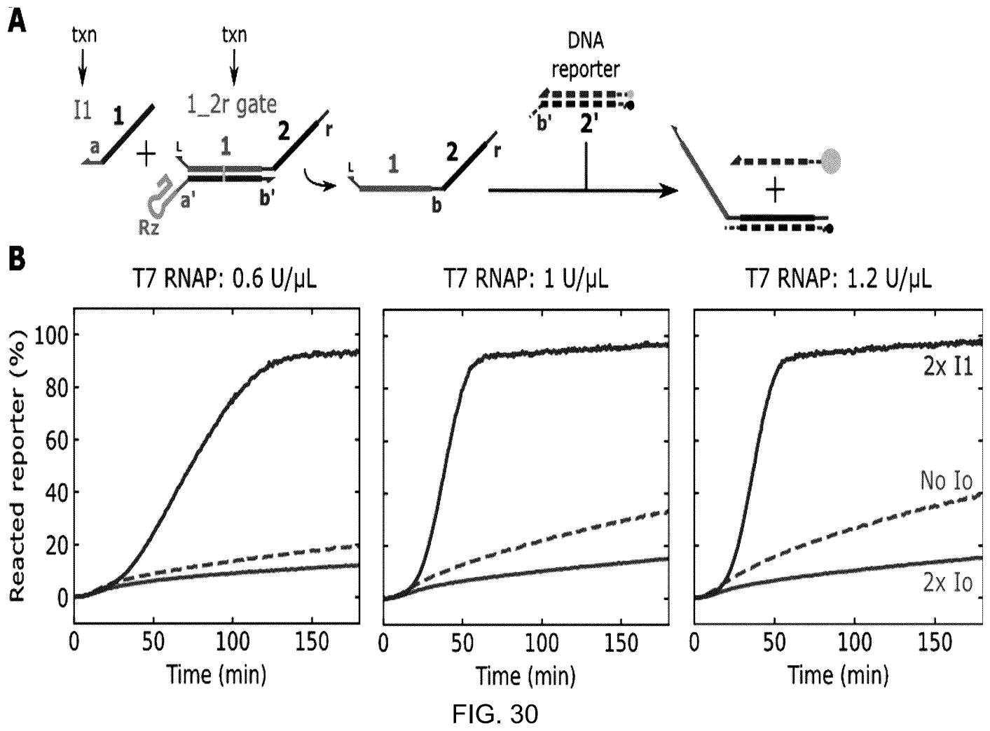

To explore ctRSD circuit kinetics, we co-transcribed the input and gate templates alongside a DNA reporter complex designed to release a fluorescent signal upon reaction with the gate output strand (FIG. 7C). We opted to use a DNA-based reporter, rather than an RNA aptamer-based reporter, because the DNA reporter is easily calibrated to output concentration for modeling. To be stable at 37° C., we designed the reporter with a 16 base duplex. The 5′ end of the 1_2 gate was extended to include the full complement of the reporter (1_2r gate) to ensure an irreversible reaction. We fixed the 1_2r gate template concentration and varied the 11 template concentration. To ensure the same transcriptional load for comparison, a template that produced an unreactive input (Io) was added to maintain the same total input template concentration across samples (Methods). As expected from mass action kinetics, increasing concentrations of the 11 template resulted in faster reaction kinetics (FIG. 7D). Further, separate transcription of the gate and input followed by mixing and addition of the DNA reporter exhibited kinetics consistent with strand displacement (FIG. 26). A gate with a mutant ribozyme that cannot cleave resulted in >3-fold slower output production (FIG. 28). Transcription of the 1_2r gate with only Io resulted in ˜20% of the maximum DNA reporter signal, indicating a slow leak reaction (FIG. 7D). The magnitude of this leak depended on T7 RNAP and total template concentrations (FIG. 30).

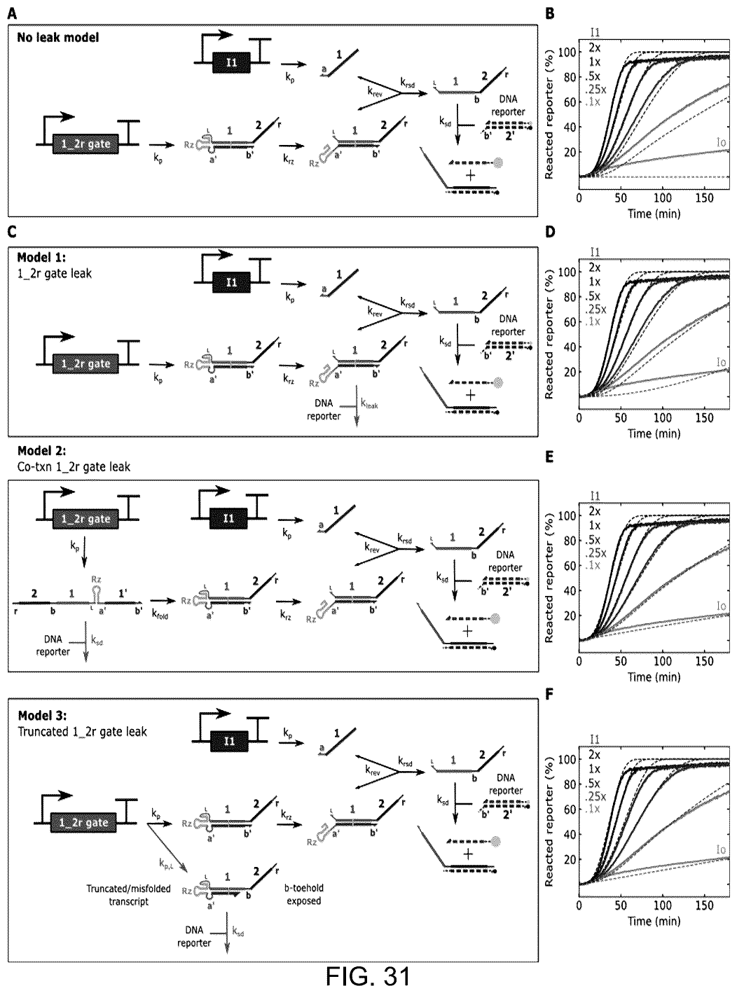

We next investigated whether a mass action kinetic model of coupled transcription, ribozyme cleavage, and RNA strand displacement could recapitulate the kinetics observed in ctRSD circuit experiments. For model parameters, we used the ribozyme cleavage rate that we measured (FIG. 22) and estimated order of magnitude strand displacement rate constants consistent with previous literature. We calibrated the transcription rate constant for each experiment with a control sample. Our initial model did not include any terms to describe the leak observed when the 1_2r gate was transcribed without the correct input and thus could not capture that effect (FIGS. 31, A and B).

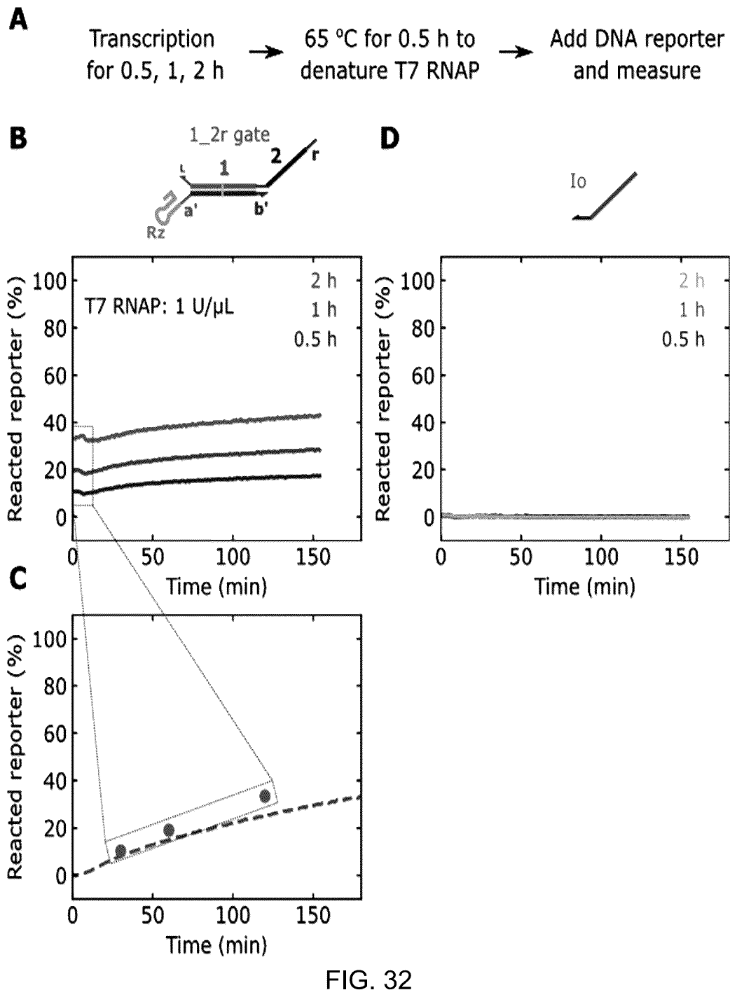

To investigate the source of the leak, we evaluated how well incorporating plausible leak pathways into the model recapitulated the experimental leak kinetics. We first evaluated a leak pathway in which the cleaved 1_2r gate could directly react with the DNA reporter via a 0 base toehold. In simulations, this model exhibited a lag time before the leak was observed, inconsistent with experiments (FIGS. 31, C and D). We next introduced a leak pathway in which the 1_2r gate could react with the DNA reporter prior to folding. In simulations, this leak pathway closely recapitulated the observed leak kinetics using a folding rate constant consistent with T7 RNAP transcription (FIGS. 31, C and E). To experimentally investigate the presence of this leak pathway, we transcribed the 1_2r gate in the absence of DNA reporter, heat denatured T7 RNAP, and then added the DNA reporter to the solution containing the folded 1_2r gate. If the leak pathway involved the unfolded 1_2r gate, no signal should be observed upon reporter addition. We found that reporter addition resulted in an instantaneous jump in fluorescence, and the magnitude of the leak signal increased with increasing 1_2r gate transcription time (FIG. 32). From these results, we reasoned the leak is not due to a reaction with the 1_2r gate prior to folding, but rather due to the presence of a 1_2r gate product that is highly reactive. Such an unintended side product could be the result of premature termination or gate misfolding events that leave the b-toehold of the gate exposed to rapidly react with the DNA reporter. We modeled this leak reaction by assuming the 1_2r gate template directly produced output at a leak transcription rate. In the model, a leak transcription rate of 3% the gate transcription rate recapitulated the experimental kinetics (FIGS. 31, C and F). We included this leak term in all subsequent simulations. With the inclusion of this leak term, the kinetic model exhibited good agreement with experimental ctRSD circuit kinetics (FIG. 7D).

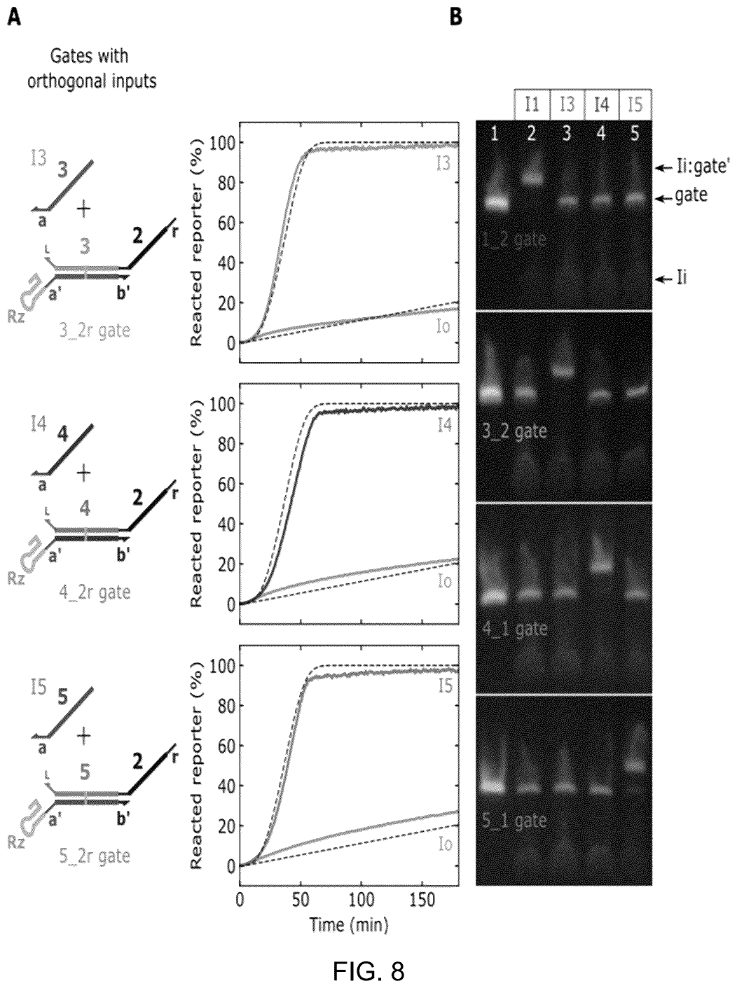

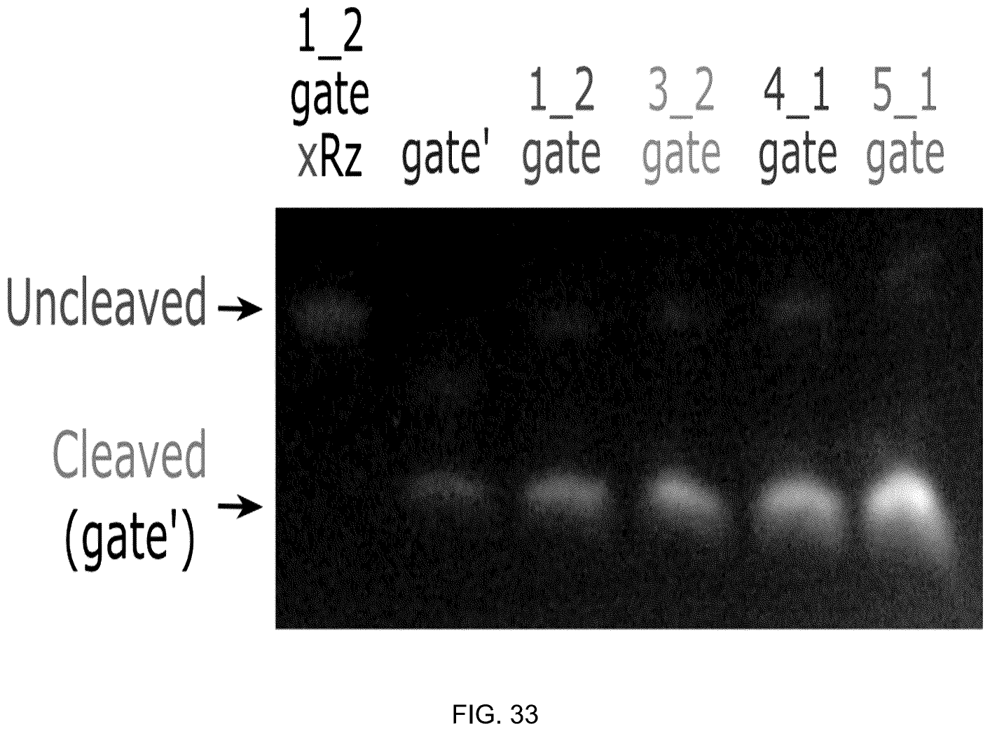

Using the same design as the 1_2r gate, we created three more ctRSD gate sequences with corresponding inputs. We reused the same input toehold sequence across gates to facilitate similar strand displacement kinetics. These gate sequences cleaved with similar efficiency as the 1_2r gate (FIG. 33) and exhibited nearly identical ctRSD circuit kinetics as the 1_2r gate (FIG. 8A). Importantly, 11, 13, 14, 15 only reacted with their designed gate (FIG. 8B), demonstrating orthogonality.

ctRSD Logic and Signal Amplification Elements

We next investigated whether ctRSD components could be programed to execute logic, signal amplification, and multi-layer cascades. To assess the predictability of ctRSD circuit design, for each circuit we built we evaluated how well our kinetic model predicted behavior. Our model assumes all ctRSD components are transcribed at the same rate and all gates cleave at the same rate. Further, we assume ctRSD components with the same toehold sequence have the same strand displacement rate constants.

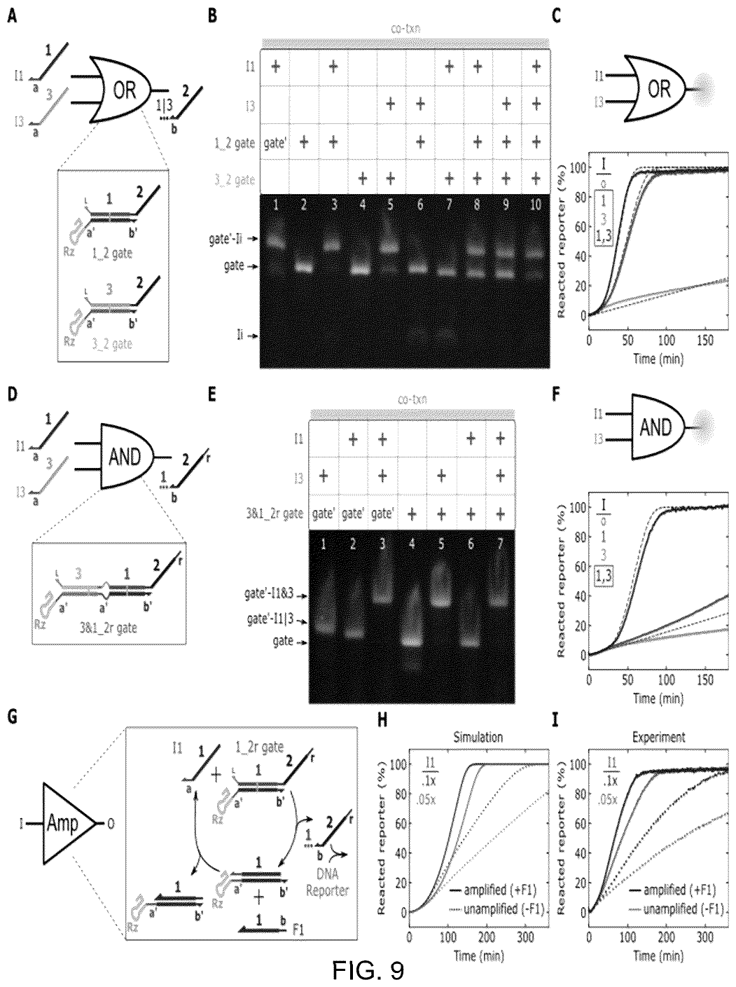

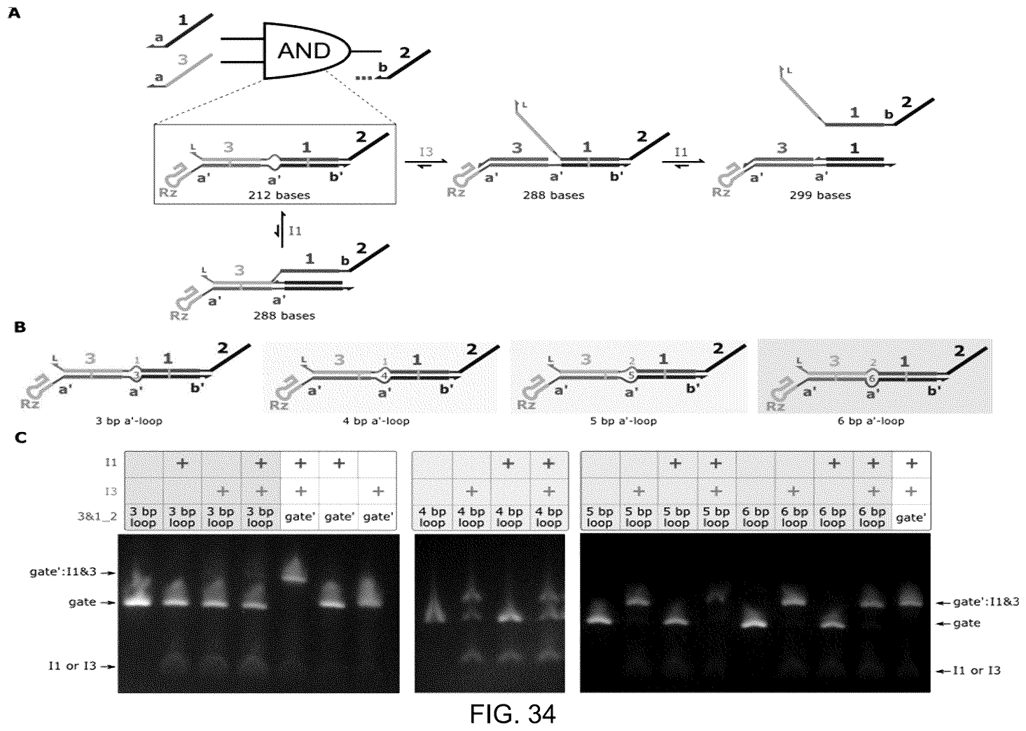

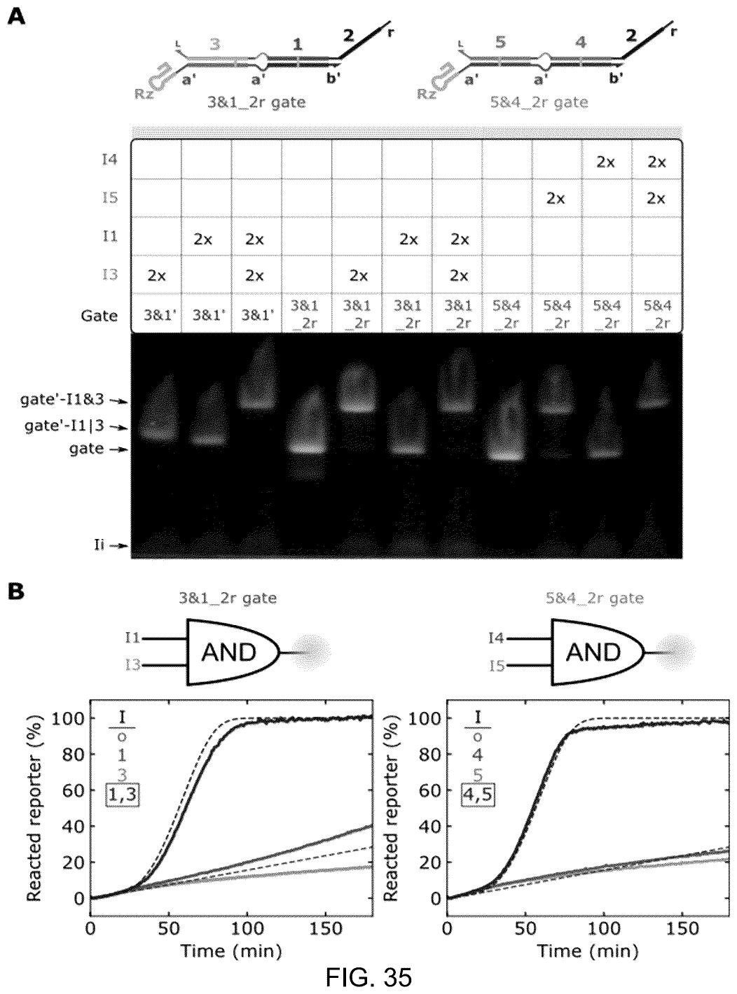

With respect to designing OR and AND logic elements, an OR element was composed of two gates that react with different inputs but release the same output (FIG. 9A). We confirmed OR functionality with native gel electrophoresis (FIG. 9B) and the DNA reporter assay (FIG. 9C). Importantly, OR element kinetics closely matched model predictions (FIG. 9C). The AND element was a gate composed of two input domains separated by an internal loop (FIG. 9D). In this design, I3 reacts with the gate to expose the toehold for I1 in the internal loop. We tested AND gates with internal loops composed of (3, 4, 5, or 6) bases of the a′-toehold. The 5 base and 6 base variants resulted in complete gate reaction with 2× input template (FIG. 34). To reduce the chance of the gate reacting with 11 alone, we chose the 5 base internal loop design. Native gel electrophoresis confirmed the AND gate reacted with I3 and I3+I1 but not with I1 alone (FIG. 9E). Similar results were observed with the DNA reporter assay, and the kinetics of output release aligned with model predictions (FIG. 9F). A second AND gate with I4 and I5 as inputs behaved similarly (FIG. 35). Our simulations suggested the AND gates exhibited 6% leak transcription compared to 3% for the single input gates. This could arise because AND gates possess two input domains in series, which may increase the likelihood of truncated or misfolded transcripts compared to single input gates.

A powerful component in strand displacement circuits is the seesaw element, which facilitates signal amplification in larger circuits. In a seesaw element, a single-stranded fuel component reacts with a 1:gate′ complex to displace the input, thus allowing multiple rounds of catalytic signal release (FIG. 9G). In DNA-based circuits, which have fixed gate and input concentrations, a seesaw element enables a gate to react completely even when the input is at a lower concentration than the gate. In ctRSD circuits, output release will eventually saturate the DNA reporter signal regardless of the input concentration. However, simulations indicated a seesaw element should decrease the time required to reach reporter saturation for low input template concentrations (FIG. 9H). When the input template was 0.05× or 0.1× the concentration of the gate template, inclusion of the fuel strand template (amplified, FIG. 9I) reduced the time to reach reporter saturation≈3-fold and ≈4-fold, respectively, compared to samples with the input template but without the fuel template (unamplified, FIG. 9I).

Multi-Layer ctRSD Cascades

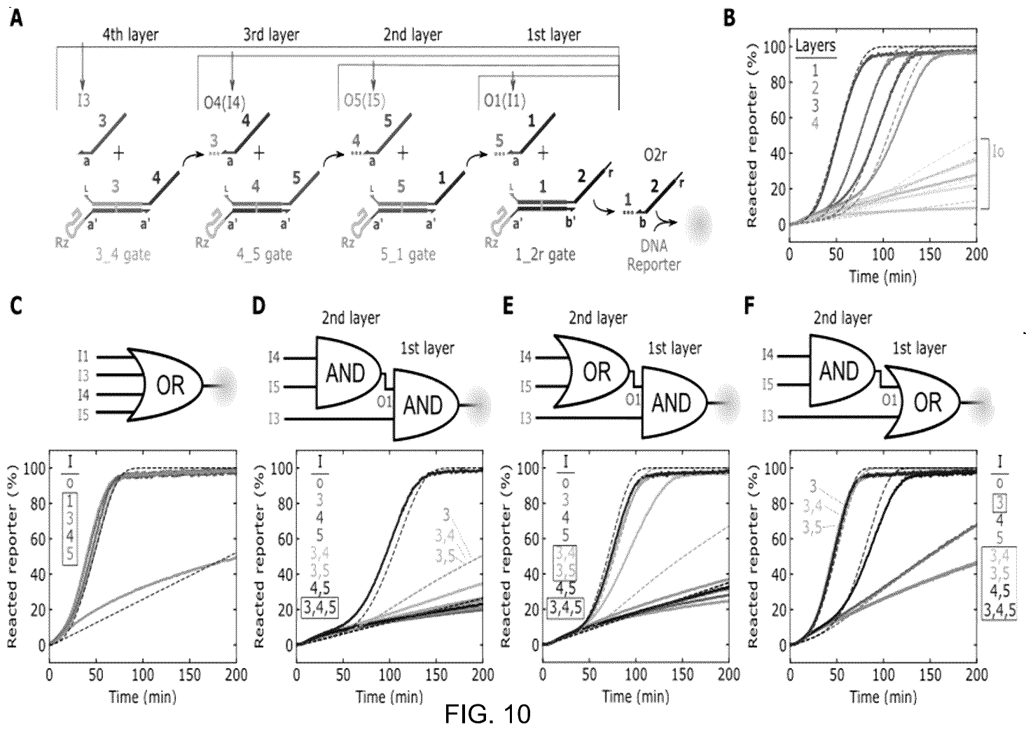

Strand displacement circuits capable of complex digital logic, pattern recognition, or temporal signal release require cascades of multi-layer signal transduction, so we next investigated whether we could program ctRSD cascades. We began by designing circuits with one to four ctRSD reaction layers in which the input and gate of the highest layer produce an output that triggers the next layer until the reporting reaction is triggered (FIG. 10A). All four multi-layer cascades exhibited kinetics in good agreement with model predictions (FIG. 10B). We next integrated ctRSD logic elements into a four-input OR circuit (FIG. 10C), a cascade of two AND gates (FIG. 10D), and two permutations of AND+OR cascades (FIGS. 10, E and F). These cascades successfully executed the designed logic operations, and the experimental kinetics generally agreed with model predictions. However, there were two minor deviations in experimental kinetics compared to model predictions.

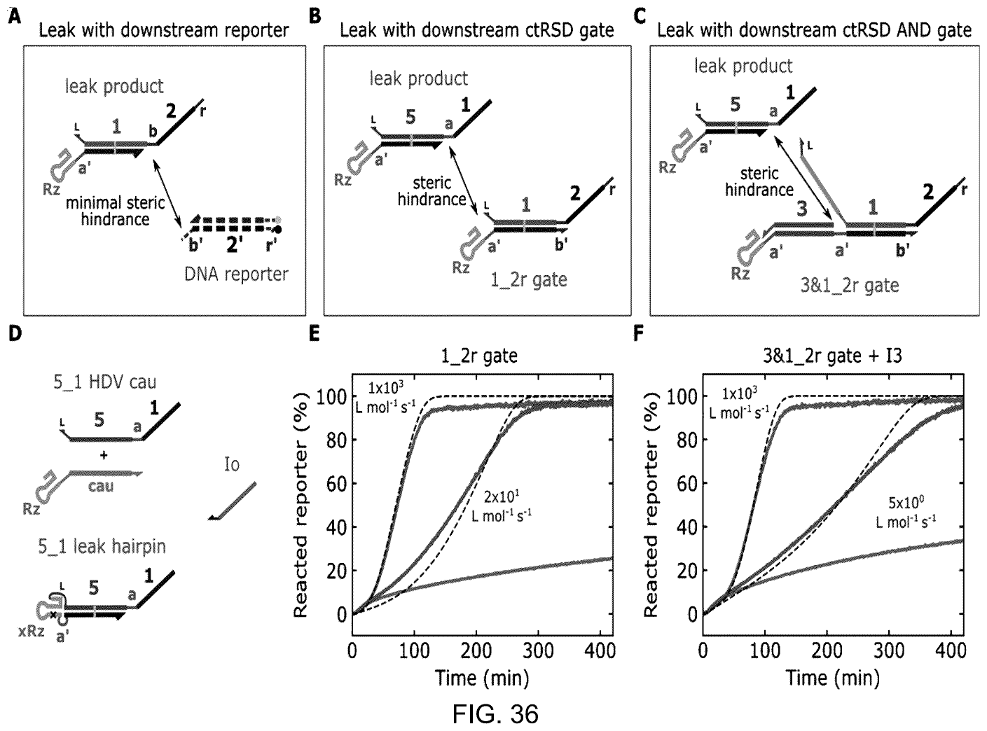

In the first deviation from the model, the two cascades in which the first layer was the 3&1_2r gate exhibited less leak than predicted when only I3 was present (FIGS. 10D and E). I3 opens the 3&1_2r gate to react with any leak products from the upstream layer in the cascades. Presumably, the 3&1_2r gate and upstream leak products reacted less than anticipated. Our model assumes leak products react with the same rate constant as their corresponding output products, but leak products are likely misfolded gates that are bulkier than single-stranded outputs. For a 13:3&1_2r complex, the region upstream of the toehold the leak product reacts with is a duplex. Thus, steric hindrance between the 13:3&1_2r complex and a leak product could result in lower leak than predicted in simulations (FIGS. 36 A-C). Similar steric hindrance between the ctRSD gate ribozyme and an upstream leak product could explain why the observed leak in multi-layer ctRSD cascades was less than predicted (FIGS. 36, A-C and FIG. 10B). In support of this hypothesis, we found the rate constant for a strand displacement reaction using an input with a hairpin directly adjacent to its toehold was nearly 100-fold lower than with a single-stranded input (FIGS. 36, D-F).

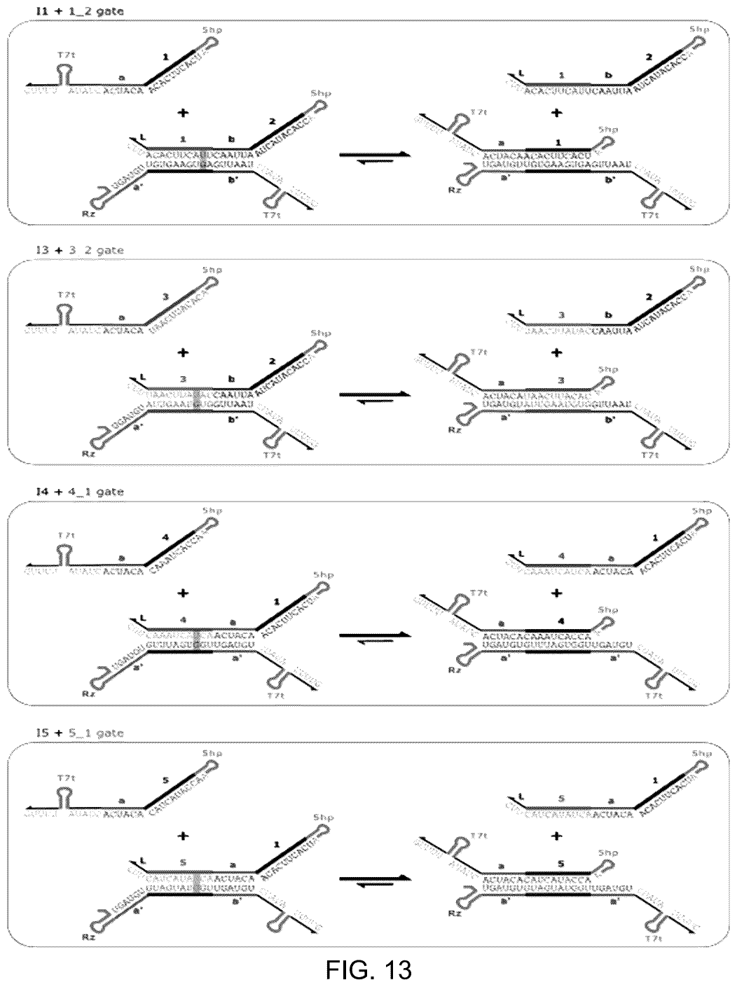

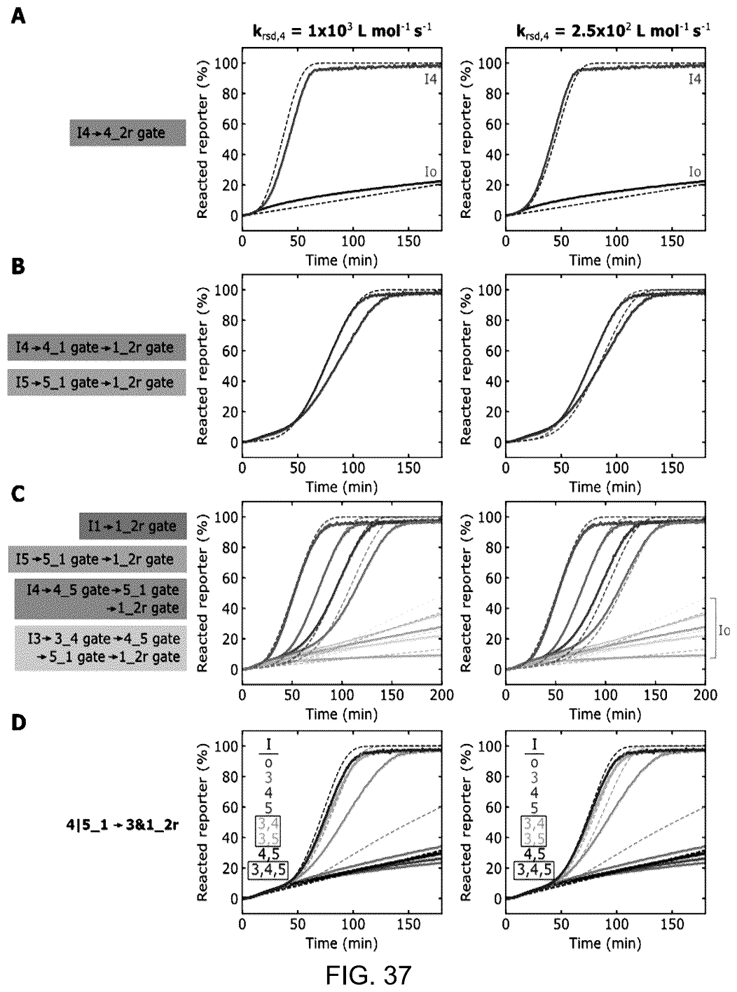

In the second deviation from the model, the I3+I4 reaction in the OR+AND cascade (FIG. 10E) was slower than predicted. This could be due to a slower strand displacement reaction for the 41 gate. The 4_1 gate itself appears to fold, cleave, and react with 14 similarly to other gates (FIG. 8 and FIG. 33), so the difference in kinetics is not due to the gate misfolding. While all gates reuse the same toehold sequence, the kinetics of the branch migration process can vary over an order of magnitude depending on the sequence. The initial branch migration sequence of the 41 gate contains a weak UA tract (FIG. 13) that could result in slower strand displacement kinetics. This mechanism is consistent with the 4_2r gate reaction being slower than gate reactions with the other three input sequences (FIG. 8A) and the four-layer ctRSD cascade being slower than predicted (FIG. 10B). Consistent with this hypothesis, reducing the 14 RNA strand displacement rate constant 2.5-fold aligned the model predictions more closely to experimental results (FIG. 37). Although these hypotheses regarding model deviations are plausible, we present analyses using the model that assumes uniform gate performance.

Varying the Toehold Lengths in ctRSD Circuits

In toehold-mediated strand displacement, kinetics can be precisely controlled by varying toehold length and sequence. Such kinetic control has been demonstrated for both DNA and RNA strand displacement. In ctRSD circuits, toehold length could also influence gate folding or ribozyme cleavage kinetics. Further, in our gate designs, the bulky ribozyme is directly adjacent to the toehold and could sterically hinder input binding. Thus, extending the gate toehold alone could influence kinetics by introducing a single-stranded spacer between the ribozyme and the sequence the input binds.

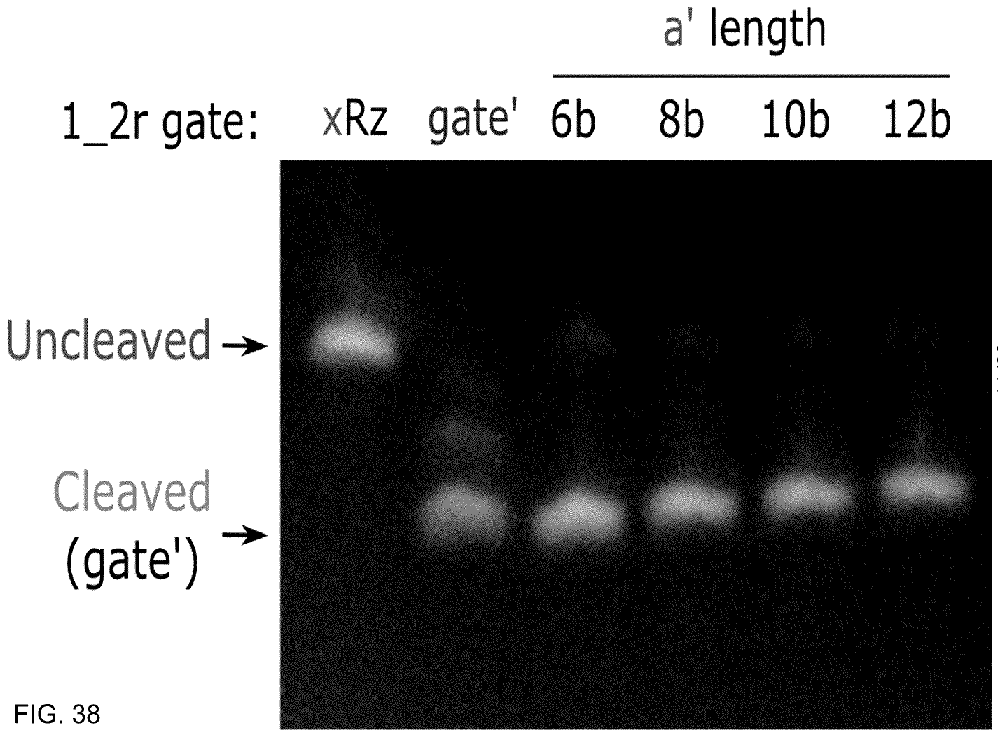

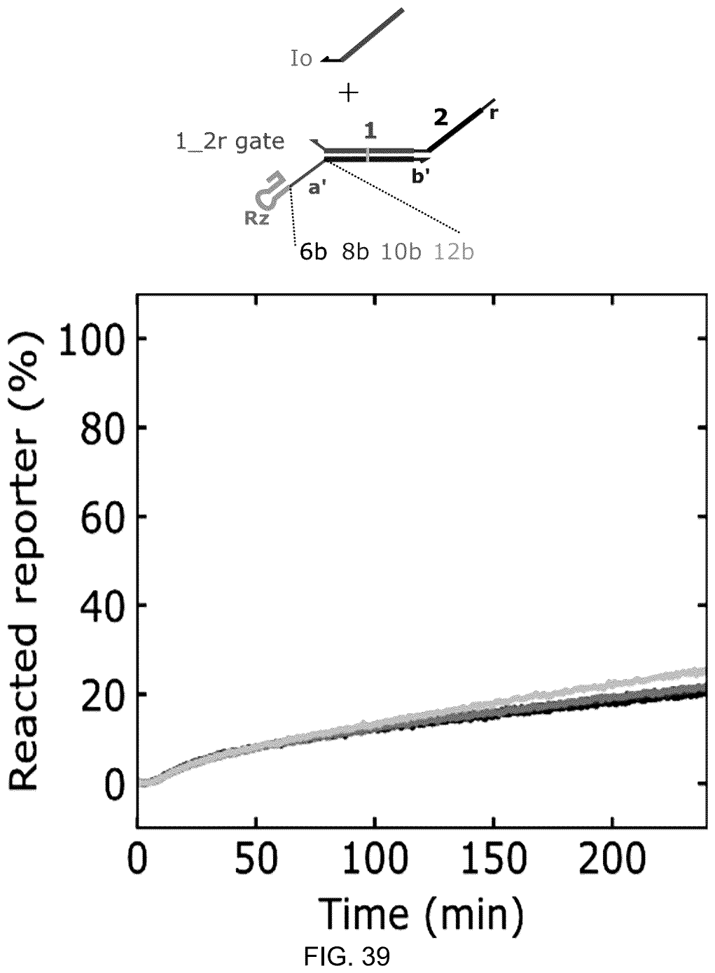

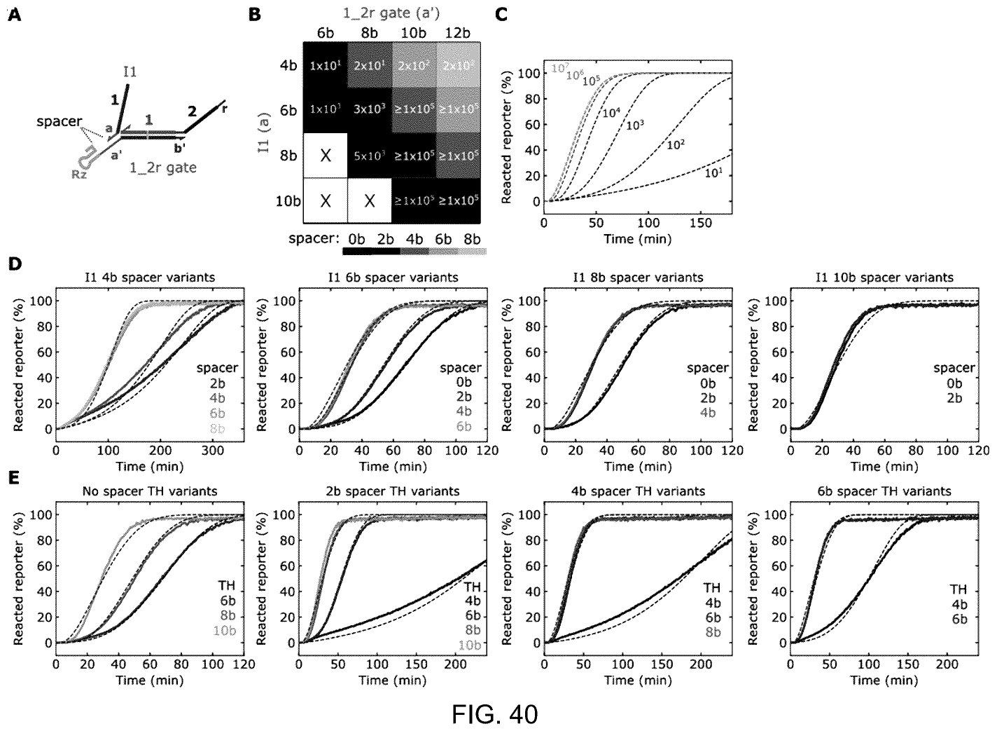

To explore the influence of toehold length on ctRSD circuit performance, we analyzed 1_2r gates with (6, 8, 10, or 12) base toeholds. These gates cleaved with similar efficiency (FIG. 38) and exhibited similar leak in the DNA reporter assay (FIG. 39), indicating proper folding and cleavage. To explore the influence of toehold length and spacer length on kinetics, we designed I1 variants possessing (4, 6, 8, or 10) base toeholds and combinatorially transcribed each input alongside a 1_2r gate possessing either a (6, 8, 10, or 12) base toehold. Varying both the toehold and spacer length allowed us to tune the strand displacement rate constant over four orders of magnitude (FIG. 40). Increasing toehold length without spacers increased the strand displacement rate. Inclusion of spacers adjacent to the ribozyme increased strand displacement kinetics for inputs with (4, 6, or 8) base toeholds. With sufficiently long spacers the reaction rate constants for all input toehold lengths aligned with predictions from DNA-based circuits, and (6, 8, or 10) base input toehold rate constants approached the theoretical maximum.

We developed scalable co-transcriptionally encoded RNA strand displacement circuits that were rationally programmed to execute logic, signal amplification, and multi-layer cascades. Integral to the development of these circuits was encoding RNA gates that co-transcriptionally folded into kinetically trapped intermediates, allowing all circuit components to be produced where they execute computations. We demonstrated the scalability and modularity of ctRSD circuits by implementing 11 single input gates and 2 AND gates in 8 different circuit topologies, all of which exhibited kinetics in agreement with our model that assumed uniform kinetic parameters. Taken together, these results indicate the robustness of our ctRSD gate design choices. Although other designs were not investigated experimentally, we believe three design choices contributed to the scalability and modularity of ctRSD circuits: 1) selecting the stable and cleavage sequence agnostic HDV ribozyme, 2) restricting the input and output sequences to C, A, or U bases, and 3) transcribing the output strand of the gates first. These choices likely reduced the chances of misfolding during transcription and facilitated proper ribozyme function across gate sequences.

We implemented the ctRSD gates with the same modular toehold exchange design (FIGS. 6, A and B) and C, A, U sequence constraints employed in state-of-the-art DNA-based circuits. In DNA computing, these designs have enabled circuits composed of >100 components to be programmed to execute complex pattern recognition tasks and implement arbitrary chemical reaction networks, functionalities not accessible with current genetically encodable RNA circuits. Thus, ctRSD circuits are poised to achieve the same scalability and functionality as the most advanced DNA-based TMSD circuits, while potentially offering improved component purity and stability at comparable costs.

Our design choices also introduce practical limitations. The C, A, U sequence constraint restricts the use of cellular RNAs composed of all four bases as inputs. Simply redesigning gates with a four letter code could make it difficult to predictably design sequences that fold correctly. To address this limitation, we envision building upstream ctRSD translation gates that modularly convert RNA inputs with a four-letter code into outputs with a three-letter code that are processed in ctRSD circuits with our prescribed design rules. In this manner, the same robust information processing circuits may be used, and translation gates with four-letter codes that function correctly could be identified by testing sequences spanning a cellular RNA of interest.

Another limitation of our design is the bulky HDV ribozyme motif left on the gates after cleavage. We found this motif influenced strand displacement kinetics unless a single-stranded spacer between the ribozyme and the toehold binding sequence was inserted. Recently, a scheme was reported for transcriptionally encoding strand displacement circuits that used a dual hammerhead ribozyme motif that excised itself after folding, and a similar multi-ribozyme strategy could be applied to ctRSD gates to remove the HDV ribozyme motif during gate production. However, in contrast to the ctRSD circuits presented here, the alternative scheme used a four-letter code and found gate performance varied with sequence. Further, toeholds switched from 5′ to 3′ between circuit layers, reducing modularity and composability. Ultimately, merging ideas from both these implementations offers routes for further optimizing ctRSD circuits.

We envision ctRSD circuits enabling many new applications in nucleic acid computing and synthetic biology. For example, the inclusion of RNases in ctRSD circuits would allow continuous circuit turnover. Circuits could then respond multiple times to changing input signals, overcoming a current limitation in DNA computing. Additionally, regulating input production with allosteric transcription factors would allow ctRSD circuits that process non-nucleic acid inputs to be readily developed for smart diagnostics. Finally, the ability to transcriptionally encode strand displacement components on DNA plasmids would allow nucleic acid computing to be employed in a number of new environments where DNA computing is limited due to degradation, e.g. in blood samples, cell-free lysates, or inside living cells. In vivo, fluorescent RNA aptamers or RNA regulators that transduce RNA signals into fluorescent protein production could track ctRSD circuit dynamics. Further, ctRSD circuit outputs could regulate protein expression through existing RNA technologies, allowing ctRSD circuits to control cellular function. Adopting ctRSD circuits for these diverse applications will require overcoming challenges in controlling expression, degradation, and cleavage rates in vivo. These issues could be addressed by optimizing 5′ hairpins to tune expression levels or increase RNA stability, as well as exploring HDV ribozyme variants. Ultimately, ctRSD circuits are poised to be a versatile, enabling technology across many synthetic biology platforms.

### Materials and Methods

### DNA and Materials

DNA transcription templates were ordered as gBlock gene fragments from Integrated DNA Technologies (IDT), amplified via polymerase chain reaction (PCR) with Phusion High-Fidelity PCR Master Mix (Cat #: F531 L) from ThermoFisher Scientific, and purified using Qiagen PCR clean-up kits. All DNA oligo primers were ordered from IDT with standard desalting. For in vitro transcription experiments T7 RNA polymerase (RNAP) and ribonucleotide triphosphates (NTPs) were ordered from ThermoFisher Scientific (Cat #: R0481). DNase I (Cat #: M0303S) was purchased from New England Biolabs (NEB). 4% agarose EX E-gels were purchased from ThermoFisher Scientific (Cat #: G401004). All chemicals were purchased from Sigma Aldrich.

### Transcription Template Preparation

All transcription templates were prepared by PCR of 0.2 ng/μL of gBlock DNA with Phusion High-Fidelity PCR Master Mix and 0.5 μmol/L of forward and reverse primers. PCR was conducted for 30 cycles with a 30 s 98° C. denaturing step, a 30 s 60° C. primer annealing step, and a 30 s 72° C. extension step. A 3 min 72° C. final extension step was executed at the end of the program. Following PCR amplification, the samples were purified with Qiagen PCR clean-up kits and eluted in Qiagen Buffer EB (10 mmol/L Tris-HCl, pH 8.5).

### RNA Agarose Gel Electrophoresis

4% agarose EX E-gels were used for all RNA gel electrophoresis experiments. These gels are pre-stained with SYBR Gold for fluorescence imaging. Electrophoresis was conducted on a E-gel powerbase, and all E-gels were imaged using the E-gel power snap camera (ThermoFisher Scientific, Cat #: G8200). Unless otherwise stated, to prepare RNA for gel electrophoresis, DNA templates were transcribed at 37° C. for 30 min in transcription conditions (see Characterization of RNA strand displacement with in vitro transcription) with 0.6 U/μL T7 RNAP. To stop transcription, CaCl₂ (final concentration (1 to 1.5) mmol/L) and DNase I (final concentration (0.1 to 0.2) U/μL) were added to degrade the DNA templates. After DNase I addition, the samples were left at 37° C. for (0.5 to 2) h, and subsequently analyzed with gel electrophoresis. For native gels, the gels were sandwiched between icepacks to keep the gels cool during electrophoresis and were run for (45 to 60) min prior to imaging. Integrated band intensities were quantified in gel images using the Gel Analysis Tool in ImageJ as previously described. For denaturing gels, prior to electrophoresis, a solution of 100% formamide, 36 mmol/L EDTA was mixed 1:1 by volume with the samples and the samples were heated to 90° C. for 5 min. The samples were then immediately loaded on gels for electrophoresis and run for (20 to 30) min before imaging. Gel images were not post processed, any brightness and contrast adjustments were executed during image acquisition and were thus applied uniformly to the images to aid visualization.

Characterization of RNA Strand Displacement with a Fluorescence DNA Reporter

The in vitro transcription reactions with DNA reporter complexes were conducted in transcription buffer prepared in house (40 mmol/L Tris-HCl-pH 7.9, 6 mmol/L MgCl₂, 10 mmol/L dithiothreitol (DTT), 10 mmol/L NaCl, and 2 mmol/L spermidine) supplemented with 2 mmol/L final concentration of each NTP type (ATP, UTP, CTP, GTP). All transcription reactions were conducted at 37° C. Unless otherwise stated, 500 nmol/L of DNA reporter was used. For in vitro transcription reactions, all components other than T7 RNAP were mixed and tracked in the plate reader for 15 min to 60 min prior to adding T7 RNAP. Addition of T7 RNAP, followed by mixing, corresponded to t=0 min in in vitro transcription experiments. The time to mix T7 RNAP into all samples for an experiment was less than one min. In our experiments, the T7 RNAP concentration varied depending on the total concentration of DNA templates present. To compare the response of a given ctRSD circuit to different input template concentrations or a different number of input templates, the same total template concentration was used across all reactions to ensure the same transcriptional load across samples. An input template (Io) that produces an RNA that does not interact with the gates was added to maintain the template concentration across samples. TABLE 4 contains the concentrations of DNA templates (including Io) and T7 RNAP used in each experiment.

### Transcription Rate Calibration and Sample Variability

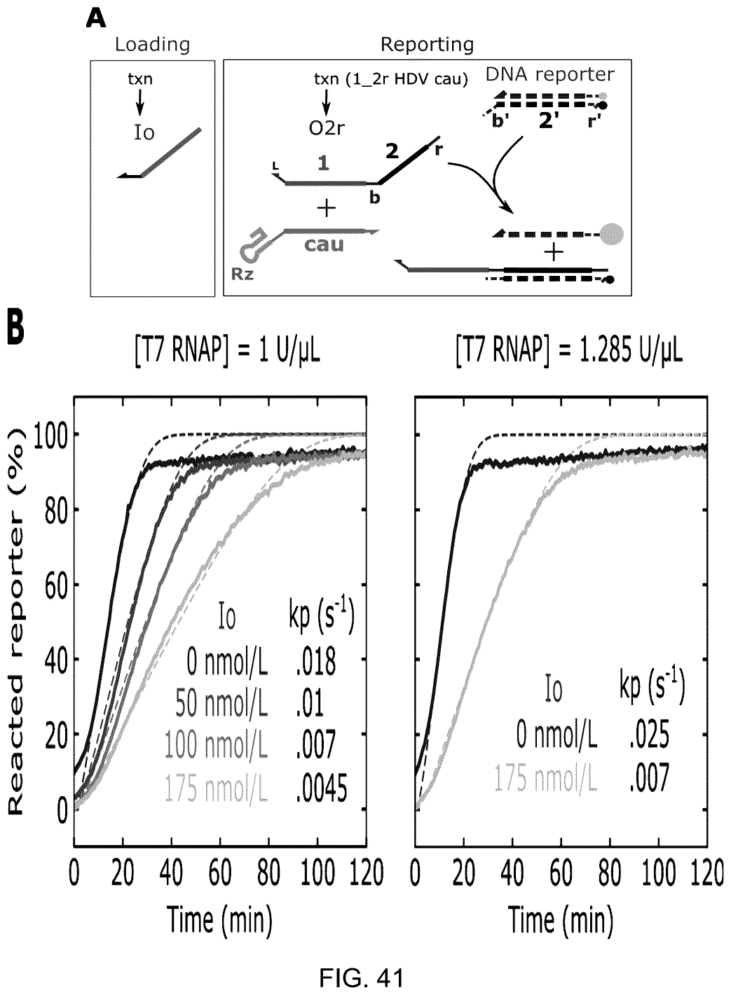

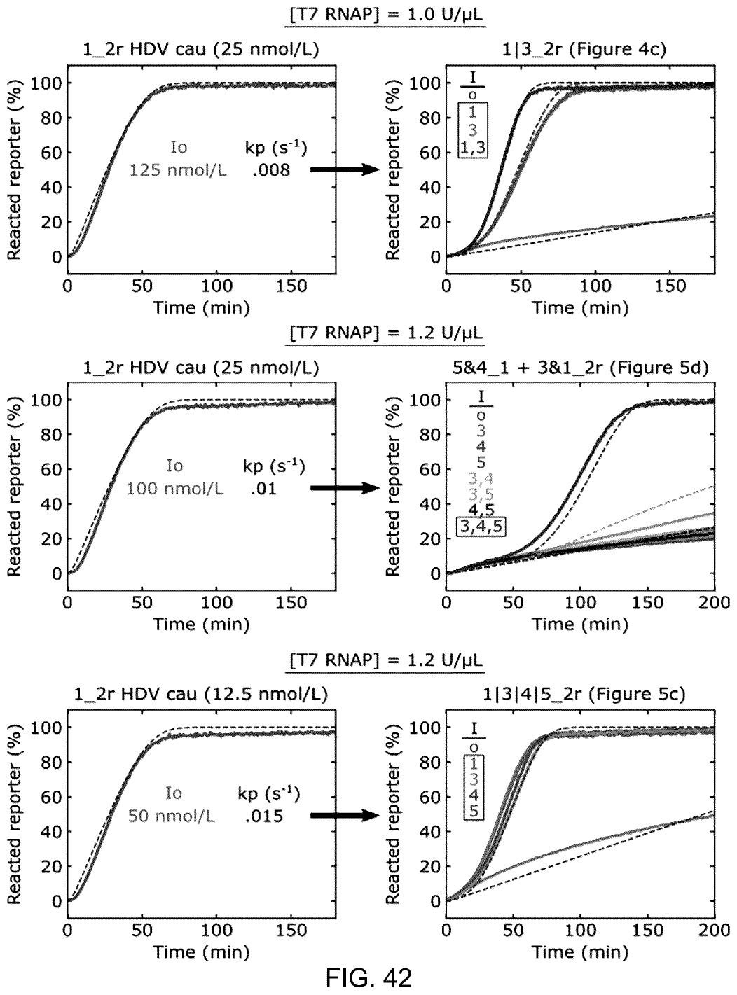

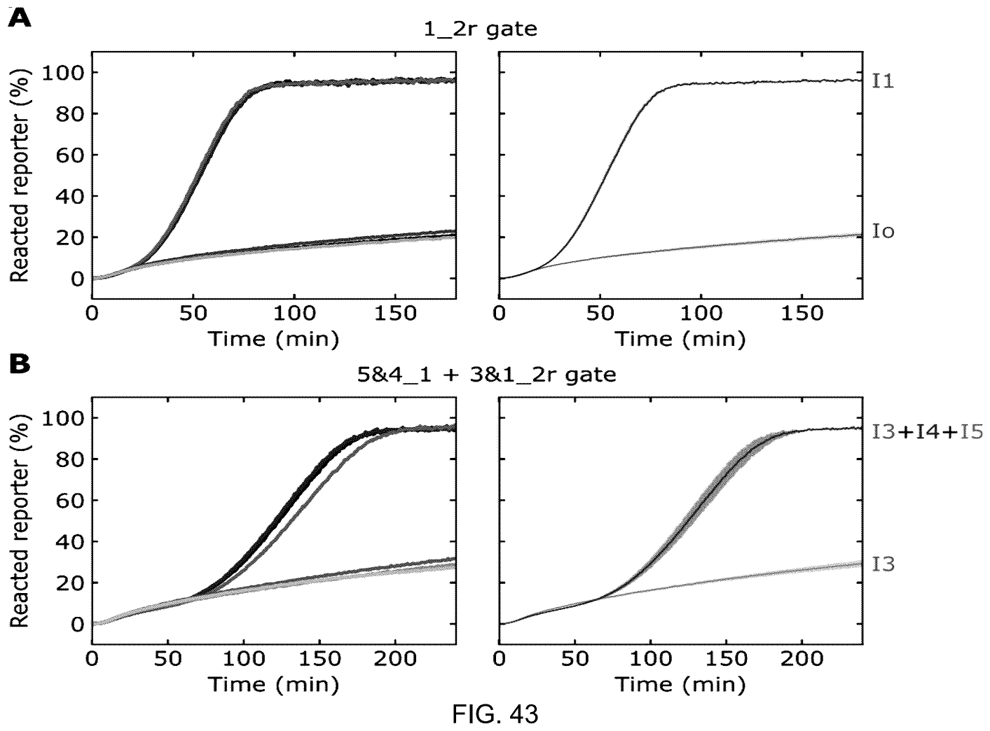

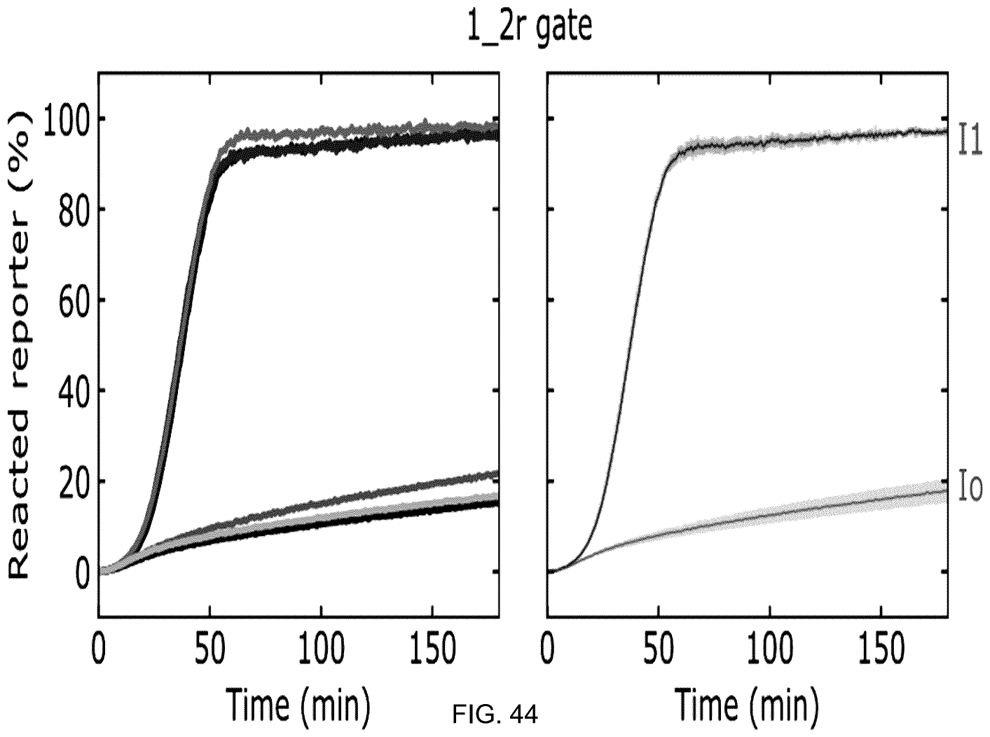

In these experiments, the transcription rate depended on the concentration of T7 RNAP and the total concentration of DNA templates (FIG. 41). Further, variability of T7 RNAP activity across manufacturer lots was expected to be the primary source of variation in our experiments. To calibrate for these effects, we developed a transcription rate reference sample (FIG. 41). This reference sample tracked transcription with a template that constitutively expressed the 1_2r strand and contained the same T7 RNAP lot and concentration as the experimental samples on a given day. Additionally, the Io template was added so the total template concentration equaled that of the experimental samples. The reference sample calibrated the first order transcription rate constant chosen for simulations (FIG. 42), thus accounting for variation in T7 RNAP activity when assessing how well experimental results agreed with model predictions. To estimate the variability in ctRSD reaction measurements introduced during sample preparation, we conducted reactions between the 1_2r gate and either I1 or Io in triplicate in the DNA reporter assay. Each reaction was prepared independently using the same transcription template, NTP, buffer, and T7 RNAP stocks. These replicates exhibited a standard deviation of <1.5% from the mean value at each time point (FIG. 43). A variability of <5% standard deviation was observed for the AND gate cascade in FIG. 10D (FIG. 43). Additionally, reactions between the 1_2r gate and either I1 or Io performed on different days exhibited <3% standard deviation (FIG. 44). We therefore assumed a conservative variability of <5% generalized to ctRSD circuits. For the small circuits studied here, we do not expect this level of variability to influence our conclusions and, unless otherwise stated, DNA reporter experiments were conducted with a single experimental replicate.

### Fluorescence Data Acquisition and Normalization

BioTek Synergy Neo2 plate readers were used track in vitro transcription reactions. Reactions were typically conducted in 70 μL volumes in Greiner μClear 96-well plates (Cat #: 655096) read from the bottom. The DNA reporter complex was labeled with a HEX dye which was tracked with excitation: 524 nm (20 nm bandwidth), emission: 565 nm (20 nm bandwidth), and a gain of 85. Fluorescence readings were taken every 46 s. In a typical experiment, fluorescence readings were taken for (25 to 45) min before T7 RNAP was added to initiate the reactions. At the end of most experiments, an excess (2.5 μmol/L) of a DNA version of the O2r strand was added to each sample to obtain an internal maximum DNA reporter fluorescence value. Fluorescence data was then normalized as:

$$Reacted reporter ( \% ) = \frac{fluorescence - min ( fluorescence )}{max ( fluorescence ) - min ( fluorescence )} * 100$$

If the DNA O2r strand was not added, a control well in which the ctRSD reaction had saturated the reporter signal served as a max value for normalization.

### Sequences, Schematics, and Control Transcripts

TABLE 1 shows DNA sequences used for this Example. All transcription templates were ordered as gBlock gene fragments from IDT. All primers were ordered without purification from IDT. For the input and fuel templates the last 30 lower case bases were added to bring the sequence above 125 bases to order as gBlocks. The PCR product resulting from the T7fwd and T7rev primers does not include this sequence. The T7 RNAP promoter sequence is underlined in all sequences. Black highlighted bases indicate bases that were mutated from a C to a T to render the HDV ribozyme catalytically inactive. Two terminators that differ in their first base were used to prevent undesired secondary structure.

TABLE 1

| | | |
|---|---|---|
| SEQUENCE NAME | SEQUENCE | SEQ ID NO. |

Common motifs

| | | |
|---|---|---|
| 5′ hairpin | GGGAGATTCGTCTCCCA | SEQ ID NO.: 1 |
| HDV ribozyme | TTCGGGTCGGCATGGCATCT | SEQ ID NO.: 2 |
| | CCACCTCCTCGCGGTCCGAC | |
| | CTGGGCTACTTCGGTAGGCT | |
| | AAGGGAG | |
| xRz HDV ribozyme | TTCGGGTCGGCATGGCATCT | SEQ ID NO.: 3 |
| | CCACCTCCTCGCGGTCCGAC | |
| | CTGGGCTACTTCGGTAGGTT | |
| | AAGGGAG | |
| T7 terminator 1 | CTATAACCCCTTGGGGCCTC | SEQ ID NO.: 4 |
| | TAAACGGGTCTTGAGGGGTT | |
| | TTTTG | |
| T7 terminator 2 | ATATAACCCCTTGGGGCCTC | SEQ ID NO.: 5 |
| | TAAACGGGTCTTGAGGGGTT | |
| | TTTTG | |

Inputs/Fuels

| | | |
|---|---|---|
| Io | TTCTAATACGACTCACTATAG | SEQ ID NO.: 6 |
| | GGAGATTCGTCTCCCAATCA | |
| | ATAACACACATACTATAACCC | |
| | CTTGGGGCCTCTAAACGGGT | |
| | CTTGAGGGGTTTTTTGCTGAA | |
| | AGGAGGAACTATATCCGGAT | |
| | ATCCC | |
| I1 | TTCTAATACGACTCACTATAG | SEQ ID NO.: 7 |
| | GGAGATTCGTCTCCCATCAC | |
| | TTCACAACATCACTATAACCC | |
| | CTTGGGGCCTCTAAACGGGT | |
| | CTTGAGGGGTTTTTTGCTGAA | |
| | AGGAGGAACTATATCCGGAT | |
| | ATCCC | |
| I3 | TTCTAATACGACTCACTATAG | SEQ ID NO.: 8 |
| | GGAGATTCGTCTCCCACACA | |
| | TTCAATACATCACTATAACCC | |
| | CTTGGGGCCTCTAAACGGGT | |
| | CTTGAGGGGTTTTTTGCTGAA | |
| | AGGAGGAACTATATCCGGAT | |
| | ATCCC | |
| I4 | TTCTAATACGACTCACTATAG | SEQ ID NO.: 9 |
| | GGAGATTCGTCTCCCAACCA | |
| | CTAAACACATCACTATAACCC | |
| | CTTGGGGCCTCTAAACGGGT | |
| | CTTGAGGGGTTTTTTGCTGAA | |
| | AGGAGGAACTATATCCGGAT | |
| | ATCCC | |
| I5 | TTCTAATACGACTCACTATAG | SEQ ID NO.: 10 |
| | GGAGATTCGTCTCCCTACCA | |
| | TACTACACATCACTATAACCC | |
| | CTTGGGGCCTCTAAACGGGT | |
| | CTTGAGGGGTTTTTTGCTGAA | |
| | AGGAGGAACTATATCCGGAT | |
| | ATCCC | |
| F1 | TTCTAATACGACTCACTATAG | SEQ ID NO.: 11 |
| | GGAGATTCGTCTCCCAATTAA | |
| | CTCACTTCACACTTTAACCCC | |
| | TTGGGGCCTCTAAACGGGTC | |
| | TTGAGGGGTTTTTTGCTGAAA | |
| | GGAGGAACTATATCCGGATA | |
| | TCCC | |
| I1 (4 base toehold) | TTCTAATACGACTCACTATAG | SEQ ID NO.: 12 |
| | GGAGATTCGTCTCCCATCAC | |
| | TTCACAACATATATAACCCCT | |
| | TGGGGCCTCTAAACGGGTCT | |
| | TGAGGGGTTTTTTGCTGAAA | |
| | GGAGGAACTATATCCGGATA | |
| | TCCC | |
| I1 (8 base toehold) | TTCTAATACGACTCACTATAG | SEQ ID NO.: 13 |
| | GGAGATTCGTCTCCCATCAC | |
| | TTCACAACATCATAATATAAC | |
| | CCCTTGGGGCCTCTAAACGG | |
| | GTCTTGAGGGGTTTTTTGCTG | |
| | AAAGGAGGAACTATATCCGG | |
| | ATATCCC | |
| I1 (10 base toehold) | TTCTAATACGACTCACTATAG | SEQ ID NO.: 14 |
| | GGAGATTCGTCTCCCATCAC | |
| | TTCACAACATCATACTATATA | |
| | ACCCCTTGGGGCCTCTAAAC | |
| | GGGTCTTGAGGGGTTTTTTG | |
| | XTGAAAGGAGGAACTATATC | |
| | CGGATATCCC | |
| Ioa | TTCTAATACGACTCACTATAG | SEQ ID NO.: 15 |
| | GGAGATTCGTCTCCCAATCA | |
| | ATAACAACATCACTATAACCC | |
| | CTTGGGGCCTCTAAACGGGT | |
| | CTTGAGGGGTTTTTTGCTGAA | |
| | AGGAGGAACTATATCCGGAT | |
| | ATCCC | |
| I1x | TTCTAATACGACTCACTATAG | SEQ ID NO.: 16 |
| | GGAGATTCGTCTCCCATCAC | |
| | TTCACACACATACTATAACCC | |
| | CTTGGGGCCTCTAAACGGGT | |
| | CTTGAGGGGTTTTTTGCTGAA | |
| | AGGAGGAACTATATCCGGAT | |
| | ATCCC | |

Gates

| | | |
|---|---|---|
| unfolded xRz gate | TTCTAATACGACTCACTATAG | SEQ ID NO.: 17 |
| | GGAGATTCGTCTCCCAACATT | |
| | ACATAATCAAATACATACATA | |
| | TTCGGGTCGGCATGGCATCT | |
| | CCACCTCCTCGCGGTCCGAC | |
| | CTGGGCTACTTCGGTAGGTT | |
| | AAGGGAGTGATGTTGTGAAG | |
| | TTAGTTAATCTATAACCCCTT | |
| | GGGGCCTCTAAACGGGTCTT | |
| | GAGGGGTTTTTTG | |
| 1_2 xRz gate | TTCTAATACGACTCACTATAG | SEQ ID NO.: 18 |
| | GGAGATTCGTCTCCCACCAC | |
| | ATACTAATTAACTTACTTCAC | |
| | ATTCGGGTCGGCATGGCATC | |
| | TCCACCTCCTCGCGGTCCGA | |
| | CCTGGGCTACTTCGGTAGGT | |
| | TAAGGGAGTGATGTTGTGAA | |
| | GTTAGTTAATCTATAACCCCT | |
| | TGGGGCCTCTAAACGGGTCT | |
| | TGAGGGGTTTTTTG | |
| 1_2r xRz gate | TTCTAATACGACTCACTATAG | SEQ ID NO.: 19 |
| | GGAGATTCGTCTCCCACTAC | |
| | ATCCACATACTAATTAACTTA | |
| | CTTCACATTCGGGTCGGCAT | |
| | GGCATCTCCACCTCCTCGCG | |
| | GTCCGACCTGGGCTACTTCG | |
| | GTAGGTTAAGGGAGTGATGT | |
| | TGTGAAGTGAGTTAATCTATA | |
| 1_2 HDV cau | ACCCCTTGGGGCCTCTAAAC | SEQ ID NO.: 20 |
| | GGGTCTTGAGGGGTTTTTTG | |
| | TTCTAATACGACTCACTATAG | |
| | GGAGATTCGTCTCCCACCAC | |
| | ATACTAATTAACTTACTTCAC | |
| | ATTCGGGTCGGCATGGCATC | |
| | TCCACCTCCTCGCGGTCCGA | |
| | CCTGGGCTACTTCGGTAGGC | |
| | TAAGGGAGATACACTCACTAT | |
| | CACTACTCATTCATAACCCCT | |
| | TGGGGCCTCTAAACGGGTCT | |
| | TGAGGGGTTTTTTG | |
| 1_2r HDV cau | TTCTAATACGACTCACTATAG | SEQ ID NO.: 21 |
| | GGAGATTCGTCTCCCACTAC | |
| | ATCCACATACTAATTAACTTA | |
| | CTTCACATTCGGGTCGGCAT | |
| | GGCATCTCCACCTCCTCGCG | |
| | GTCCGACCTGGGCTACTTCG | |
| | GTAGGCTAAGGGAGATACAC | |
| | TCACTATCACTACTCATTCAT | |
| | AACCCCTTGGGGCCTCTAAA | |
| | CGGGTCTTGAGGGGTTTTTT | |
| | G | |
| 1_2 gate HP Rz | TTCTAATACGACTCACTATAG | SEQ ID NO.: 22 |
| | GGAGATTCGTCTCCCACCAC | |
| | ATACTAATTAACTTACTTCAC | |
| | ATACCCTCACAGTCCAGTGAT | |
| | TCGTCACTGAGAAGTGAACC | |
| | AGAGAAACACACGTTGTGGT | |
| | ATATTACCTGGTCTGATGTTG | |
| | TGAAGTGAGTTAATCTATAAC | |
| | CCCTTGGGGCCTCTAAACGG | |
| | GTCTTGAGGGGTTTTTTG | |
| gate′ | TTCTAATACGACTCACTATAG | SEQ ID NO.: 23 |
| | GGAGATTCGTCTCCCATTCG | |
| | GGTCGGCATGGCATCTCCAC | |
| | CTCCTCGCGGTCCGACCTGG | |
| | GCTACTTCGGTAGGCTAAGG | |
| | GAGTGATGTTGTGAAGTGAG | |
| | TTAATCTATAACCCCTTGGGG | |
| | CCTCTAAACGGGTCTTGAGG | |
| | GGTTTTTTG | |
| 1_2 gate | TTCTAATACGACTCACTATAG | SEQ ID NO.: 24 |
| | GGAGATTCGTCTCCCACCAC | |
| | ATACTAATTAACTTACTTCAC | |
| | ATTCGGGTCGGCATGGCATC | |
| | TCCACCTCCTCGCGGTCCGA | |
| | CCTGGGCTACTTCGGTAGGC | |
| | TAAGGGAGTGATGTTGTGAA | |
| | GTGAGTTAATCTATAACCCCT | |
| | TGGGGCCTCTAAACGGGTCT | |
| | TGAGGGGTTTTTTG | |
| 1_2r gate | TTCTAATACGACTCACTATAG | SEQ ID NO.: 25 |
| | GGAGATTCGTCTCCCACTAC | |
| | ATCCACATACTAATTAACTTA | |
| | CTTCACATTCGGGTCGGCAT | |
| | GGCATCTCCACCTCCTCGCG | |
| | GTCCGACCTGGGCTACTTCG | |
| | GTAGGCTAAGGGAGTGATGT | |
| | TGTGAAGTGAGTTAATCTATA | |
| | ACCCCTTGGGGCCTCTAAAC | |
| | GGGTCTTGAGGGGTTTTTTG | |
| 3_2 gate | TTCTAATACGACTCACTATAG | SEQ ID NO.: 26 |
| | GGAGATTCGTCTCCCACCAC | |
| | ATACTAATTAACCATATTCAAT | |
| | TTCGGGTCGGCATGGCATCT | |
| | CCACCTCCTCGCGGTCCGAC | |
| | CTGGGCTACTTCGGTAGGCT | |
| | AAGGGAGTGATGTATTGAAT | |
| | GTGGTTAATCTATAACCCCTT | |
| | GGGGCCTCTAAACGGGTCTT | |
| | GAGGGGTTTTTTG | |
| 3_2r gate | TTCTAATACGACTCACTATAG | SEQ ID NO.: 27 |
| | GGAGATTCGTCTCCCACTAC | |
| | ATCCACATACTAATTAACCAT | |
| | ATTCAATTTCGGGTCGGCAT | |
| | GGCATCTCCACCTCCTCGCG | |
| | GTCCGACCTGGGCTACTTCG | |
| | GTAGGCTAAGGGAGTGATGT | |
| | ATTGAATGTGGTTAATCTATA | |
| | ACCCCTTGGGGCCTCTAAAC | |
| | GGGTCTTGAGGGGTTTTTTG | |
| 4_2r gate | TTCTAATACGACTCACTATAG | SEQ ID NO.: 28 |
| | GGAGATTCGTCTCCCACTAC | |
| | ATCCACATACTAATTAACACT | |
| | ACTAAACTTCGGGTCGGCAT | |
| | GGCATCTCCACCTCCTCGCG | |
| | GTCCGACCTGGGCTACTTCG | |
| | GTAGGCTAAGGGAGTGATGT | |
| | GTTTAGTGGTGTTAATCTATA | |
| | ACCCCTTGGGGCCTCTAAAC | |
| | GGGTCTTGAGGGGTTTTTTG | |
| 5_2r gate | TTCTAATACGACTCACTATAG | SEQ ID NO.: 29 |
| | GGAGATTCGTCTCCCACTAC | |
| | ATCCACATACTAATTAACACT | |
| | ATACTACTTCGGGTCGGCAT | |
| | GGCATCTCCACCTCCTCGCG | |
| | GTCCGACCTGGGCTACTTCG | |
| | GTAGGCTAAGGGAGTGATGT | |
| | GTAGTATGGTGTTAATCTATA | |
| | ACCCCTTGGGGCCTCTAAAC | |
| | GGGTCTTGAGGGGTTTTTTG | |
| 4_1 gate | TTCTAATACGACTCACTATAG | SEQ ID NO.: 30 |
| | GGAGATTCGTCTCCCATCAC | |
| | TTCACAACATCAACTACTAAA | |
| | CTTCGGGTCGGCATGGCATC | |
| | TCCACCTCCTCGCGGTCCGA | |
| | CCTGGGCTACTTCGGTAGGC | |
| | TAAGGGAGTGATGTGTTTAGT | |
| | GGTTGATGTCTATAACCCCTT | |
| | GGGGCCTCTAAACGGGTCTT | |
| | GAGGGGTTTTTTG | |
| 5_1 gate | TTCTAATACGACTCACTATAG | SEQ ID NO.: 31 |
| | GGAGATTCGTCTCCCATCAC | |
| | TTCACAACATCAACTATACTA | |
| | CTTCGGGTCGGCATGGCATC | |
| | TCCACCTCCTCGCGGTCCGA | |
| | CCTGGGCTACTTCGGTAGGC | |
| | TAAGGGAGTGATGTGTAGTA | |
| | TGGTTGATGTCTATAACCCCT | |
| | TGGGGCCTCTAAACGGGTCT | |
| | TGAGGGGTTTTTTG | |
| 4_5 gate | TTCTAATACGACTCACTATAG | SEQ ID NO.: 32 |
| | GGAGATTCGTCTCCCAACCA | |
| | TACTACACATCAACTACTAAA | |
| | CTTCGGGTCGGCATGGCATC | |
| | TCCACCTCCTCGCGGTCCGA | |
| | CCTGGGCTACTTCGGTAGGC | |
| | TAAGGGAGTGATGTGTTTAGT | |
| | GGTTGATGTAATTAACCCCTT | |
| | GGGGCCTCTAAACGGGTCTT | |
| | GAGGGGTTTTTTG | |
| 3_4 gate | TTCTAATACGACTCACTATAG | SEQ ID NO.: 33 |
| | GGAGATTCGTCTCCCAACCA | |
| | CTAAACACATCACATATTCAA | |
| | TTTCGGGTCGGCATGGCATC | |
| | TCCACCTCCTCGCGGTCCGA | |
| | CCTGGGCTACTTCGGTAGGC | |
| | TAAGGGAGTGATGTATTGAAT | |
| | GTGTGATGTAATTAACCCCTT | |
| | GGGGCCTCTAAACGGGTCTT | |
| | GAGGGGTTTTTTG | |
| 3&1_2r gate | TTCTAATACGACTCACTATAG | SEQ ID NO.: 34 |
| | GGAGATTCGTCTCCCACTAC | |
| | ATCCACATACTAATTAACTTA | |
| | CTTCACATAATACATTCAATTT | |
| | CGGGTCGGCATGGCATCTCC | |
| | ACCTCCTCGCGGTCCGACCT | |
| | GGGCTACTTCGGTAGGCTAA | |
| | GGGAGTGATGTATTGAATGT | |
| | GTGATGTTGTGAAGTGAGTTA | |
| | ATCTATAACCCCTTGGGGCC | |
| | TCTAAACGGGTCTTGAGGGG | |
| | TTTTTTG | |
| gate′ (3&1_2) | TTCTAATACGACTCACTATAG | SEQ ID NO.: 35 |
| | GGAGATTCGTCTCCCATTCG | |
| | GGTCGGCATGGCATCTCCAC | |
| | CTCCTCGCGGTCCGACCTGG | |
| | GCTACTTCGGTAGGCTAAGG | |
| | GAGTGATGTATTGAATGTGTG | |
| | ATGTTGTGAAGTGAGTTAATC | |
| | TATAACCCCTTGGGGCCTCT | |
| | AAACGGGTCTTGAGGGGTTT | |
| | TTTG | |
| 5&4_1 gate | TTCTAATACGACTCACTATAG | SEQ ID NO.: 36 |
| | GGAGATTCGTCTCCCATCAC | |
| | TTCACAACATCAACTACTAAA | |
| | CTAAACTATACTACTTCGGGT | |
| | CGGCATGGCATCTCCACCTC | |
| | CTCGCGGTCCGACCTGGGCT | |
| | ACTTCGGTAGGCTAAGGGAG | |
| | TGATGTGTAGTATGGTTGATG | |
| | TGTTTAGTGGTTGATGTCTAT | |
| | AACCCCTTGGGGCCTCTAAA | |
| | CGGGTCTTGAGGGGTTTTTT | |
| | G | |
| 5&4_2r gate | TTCTAATACGACTCACTATAG | SEQ ID NO.: 37 |
| | GGAGATTCGTCTCCCACTAC | |
| | ATCCACATACTAATTAACACT | |
| | ACTAAACTAAACTATACTACT | |
| | TCGGGTCGGCATGGCATCTC | |
| | CACCTCCTCGCGGTCCGACC | |
| | TGGGCTACTTCGGTAGGCTA | |
| | AGGGAGTGATGTGTAGTATG | |
| | GTTGATGTGTTTAGTGGTGTT | |
| | AATCTATAACCCCTTGGGGC | |
| | CTCTAAACGGGTCTTGAGGG | |
| | GTTTTTTG | |
| 3&1_2 (3bp a′-loop) | TTCTAATACGACTCACTATAG | SEQ ID NO.: 38 |
| | GGAGATTCGTCTCCCACCAC | |
| | ATACTAATTAACTTACTTCAC | |
| | ATTCACATATTCAATTTCGGG | |
| | TCGGCATGGCATCTCCACCT | |
| | CCTCGCGGTCCGACCTGGGC | |
| | TACTTCGGTAGGCTAAGGGA | |
| | GTGATGTATTGAATGTGTGAT | |
| | GTTGTGAAGTGAGTTAATCTA | |
| | TAACCCCTTGGGGCCTCTAA | |
| | ACGGGTCTTGAGGGGTTTTT | |
| | TG | |
| 3&1_2 (4bp a′-loop) | TTCTAATACGACTCACTATAG | SEQ ID NO.: 39 |
| | GGAGATTCGTCTCCCACCAC | |
| | ATACTAATTAACTTACTTCAC | |
| | ACCATACATTCAATTTCGGGT | |
| | CGGCATGGCATCTCCACCTC | |
| | CTCGCGGTCCGACCTGGGCT | |
| | ACTTCGGTAGGCTAAGGGAG | |
| | TGATGTATTGAATGTGTGATG | |
| | TTGTGAAGTGAGTTAATCTAT | |
| | AACCCCTTGGGGCCTCTAAA | |
| | CGGGTCTTGAGGGGTTTTTT | |
| | G | |
| 3&1_2 (5bp a′-loop) | TTCTAATACGACTCACTATAG | SEQ ID NO.: 40 |
| | GGAGATTCGTCTCCCACCAC | |
| | ATACTAATTAACTTACTTCAC | |
| | ATAATACATTCAATTTCGGGT | |
| | CGGCATGGCATCTCCACCTC | |
| | CTCGCGGTCCGACCTGGGCT | |
| | ACTTCGGTAGGCTAAGGGAG | |
| | TGATGTATTGAATGTGTGATG | |
| | TTGTGAAGTGAGTTAATCTAT | |
| | AACCCCTTGGGGCCTCTAAA | |
| | CGGGTCTTGAGGGGTTTTTT | |
| | G | |
| 3&1_2 (6bp a′-loop) | TTCTAATACGACTCACTATAG | SEQ ID NO.: 41 |
| | GGAGATTCGTCTCCCACCAC | |
| | ATACTAATTAACTTACTTCAC | |
| | ACCATATTCAATTTCGGGTCG | |
| | GCATGGCATCTCCACCTCCT | |
| | CGCGGTCCGACCTGGGCTAC | |
| | TTCGGTAGGCTAAGGGAGTG | |
| | ATGTATTGAATGTGTGATGTT | |
| | GTGAAGTGAGTTAATCTATAA | |
| | CCCCTTGGGGCCTCTAAACG | |
| | GGTCTTGAGGGGTTTTTTG | |
| 5_1 HDV cau | TTCTAATACGACTCACTATAG | SEQ ID NO.: 42 |
| | GGAGATTCGTCTCCCATCAC | |
| | TTCACAACATCAACTATACTA | |
| | CTTCGGGTCGGCATGGCATC | |
| | TCCACCTCCTCGCGGTCCGA | |
| | CCTGGGCTACTTCGGTAGGC | |
| | TAAGGGAGTACACTCACTATC | |
| | ACTACTCATTCATAACCCCTT | |
| | GGGGCCTCTAAACGGGTCTT | |
| | GAGGGGTTTTTTG | |
| 5_1 leak hairpin | TTCTAATACGACTCACTATAG | SEQ ID NO.: 43 |
| | GGAGATTCGTCTCCCATCAC | |
| | TTCACAACATCAACTATACTA | |
| | CTTCGGGTCGGCATGGCATC | |
| | TCCACCTCCTCGCGGTCCGA | |
| | CCTGGGCTACTTCGGTAGGT | |
| | TAAGGGAGTGATGTGTAGTA | |
| | TGGTCTATAACCCCTTGGGG | |
| | CCTCTAAACGGGTCTTGAGG | |
| | GGTTTTTTG | |
| 1_2r gate (8 base | TTCTAATACGACTCACTATAG | SEQ ID NO.: 44 |
| toehold) | GGAGATTCGTCTCCCACTAC | |
| | ATCCACATACTAATTAACTTA | |
| | CTTCACATTCGGGTCGGCAT | |
| | GGCATCTCCACCTCCTCGCG | |
| | GTCCGACCTGGGCTACTTCG | |
| | GTAGGCTAAGGGAGTATGAT | |
| | GTTGTGAAGTGAGTTAATCTA | |
| | TAACCCCTTGGGGCCTCTAA | |
| | ACGGGTCTTGAGGGGTTTTT | |
| | TG | |
| 1_2r gate (10 base | TTCTAATACGACTCACTATAG | SEQ ID NO.: 45 |
| toehold) | GGAGATTCGTCTCCCACTAC | |
| | ATCCACATACTAATTAACTTA | |
| | CTTCACATTCGGGTCGGCAT | |
| | GGCATCTCCACCTCCTCGCG | |
| | GTCCGACCTGGGCTACTTCG | |
| | GTAGGCTAAGGGAGAGTATG | |
| | ATGTTGTGAAGTGAGTTAATC | |
| | TATAACCCCTTGGGGCCTCT | |
| | AAACGGGTCTTGAGGGGTTT | |
| | TTTG | |
| 1_2r gate (12 base | TTCTAATACGACTCACTATAG | SEQ ID NO.: 46 |
| toehold) | GGAGATTCGTCTCCCACTAC | |
| | ATCCACATACTAATTAACTTA | |
| | CTTCACATTCGGGTCGGCAT | |
| | GGCATCTCCACCTCCTCGCG | |
| | GTCCGACCTGGGCTACTTCG | |
| | GTAGGCTAAGGGAGAGAGTA | |
| | TGATGTTGTGAAGTGAGTTAA | |
| | TCTATAACCCCTTGGGGCCT | |
| | CTAAACGGGTCTTGAGGGGT | |
| | TTTTTG | |

DNA oligos

| | | |
|---|---|---|
| T7fwd | TTCTAATACGACTCACTATAG | SEQ ID NO.: 47 |
| | GGAG | |
| T7rev | CAAAAAACCCCTCAAGACCC | SEQ ID NO.: 48 |
| | GTTTAG | |
| O2-nt | TTCTAATACGACTCACTATAG | SEQ ID NO.: 49 |
| | GGAGATTCGTCTCCCACCAC | |
| | ATACTAATTAACTTACTTCAC | |
| | ATTC | |
| O2-t | GAATGTGAAGTAAGTTAATTA | SEQ ID NO.: 50 |
| | GTATGTGGTGGGAGACGAAT | |
| | CTCCCTATAGTGAGTCGTATT | |
| | AGAA | |
| O2r-nt | TTCTAATACGACTCACTATAG | SEQ ID NO.: 51 |
| | GGAGATTCGTCTCCCACTAC | |
| | ATCCACATACTAATTAACTTA | |
| | CTTCACATTC | |
| O2r-t | GAATGTGAAGTAAGTTAATTA | SEQ ID NO.: 52 |
| | GTATGTGGATGTAGTGGGAG | |
| | ACGAATCTCCCTATAGTGAGT | |
| | CGTATTAGAA | |
| DNA reporter F | /5HEX/CTACATCCACATACTA | SEQ ID NO.: 53 |
| DNA reporter Q | GTTAATTAGTATGTGGATGTA | SEQ ID NO.: 54 |
| | G/3IABRQSP/ | |

ctRSD Gate Design Considerations

Different design considerations were analyzed during development of the ctRSD gates. Two methods to transcriptionally encode RNA strand displacement gates include: transcription of the two gate strands from separate transcription templates or transcription of an RNA hairpin with a ribozyme that cleaves the hairpin after folding to produce a dsRNA gate. The former method introduces a significant downstream leak reaction and was not used. Below provides analysis of four different transcription paths for producing ctRSD gates. In principle, these different transcription paths are conceptually equivalent but depend on the selected toehold directionality (5′ vs 3′) and the position of the ribozyme within the transcript. Analysis of three different self-cleaving ribozyme options for the ctRSD gates is described below.

### The Co-Transcriptional Folding Pathway

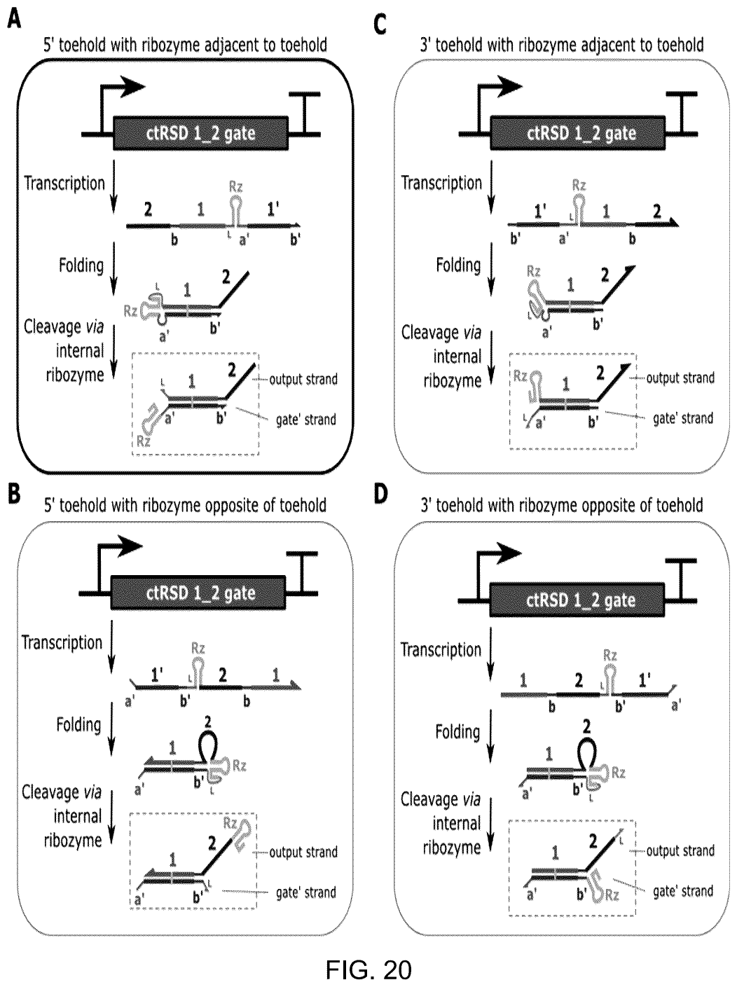

Considering the directionality of the single-stranded RNA (ssRNA) toehold that facilitates strand displacement and the placement of the self-cleaving ribozyme within the RNA transcript, there are four possible designs for ctRSD gates (FIG. 20). Previous work indicates that 5′ toeholds on RNA strand displacement gates perform better than 3′ toeholds, so we focused on designs with 5′ toeholds (FIGS. 20, A and B). The placement of the self-cleaving ribozyme influences which domains of the gate are transcribed first. For example, placing the ribozyme adjacent to the 5′ toehold results in transcription of the output region (2-, b-, and 1-domains) of the gate first, while placing the ribozyme on the opposite side of the transcript results in transcription of the gate′ strand first. In many DNA strand displacement circuits the output sequences of the DNA gates are constrained to a 3 letter code (C, A, or, T). This constraint reduces the possibility of unwanted secondary structure from forming and preventing output strands in larger circuits from hybridizing with each other (crosstalk). We adopted the same sequence constraints in our RNA gate designs, limiting the gate output sequences (2-, b-, and 1-domains) to only C, A, or U bases. This constraint is particularly important for RNA circuits because G-U wobble base pairings are more energetically favored in RNA than G-T wobble pairings in DNA. Thus, even output sequences constrained to G, A, U bases could fold into undesired secondary structures. In the ctRSD gate design in which the self-cleaving ribozyme is placed opposite of the gate toehold (FIG. 20B), the gate′ strand (a′-, 1′-, and b′-domains), whose sequence would be composed of G, A, U bases, would be transcribed first. Given that co-transcriptional folding of RNA is much faster than transcription, transcription of the a′-, 1′-, and b′-domains first could result in undesired secondary structure in the transcript before the complementary 1- and b-domains are transcribed, hindering the correct gate formation. For these reasons, we chose the RNA gate design in which the 2-, b-, and 1-domains (composed of only C, A, U bases) are transcribed first (FIG. 20A).

### Self-Cleaving Ribozyme Selection

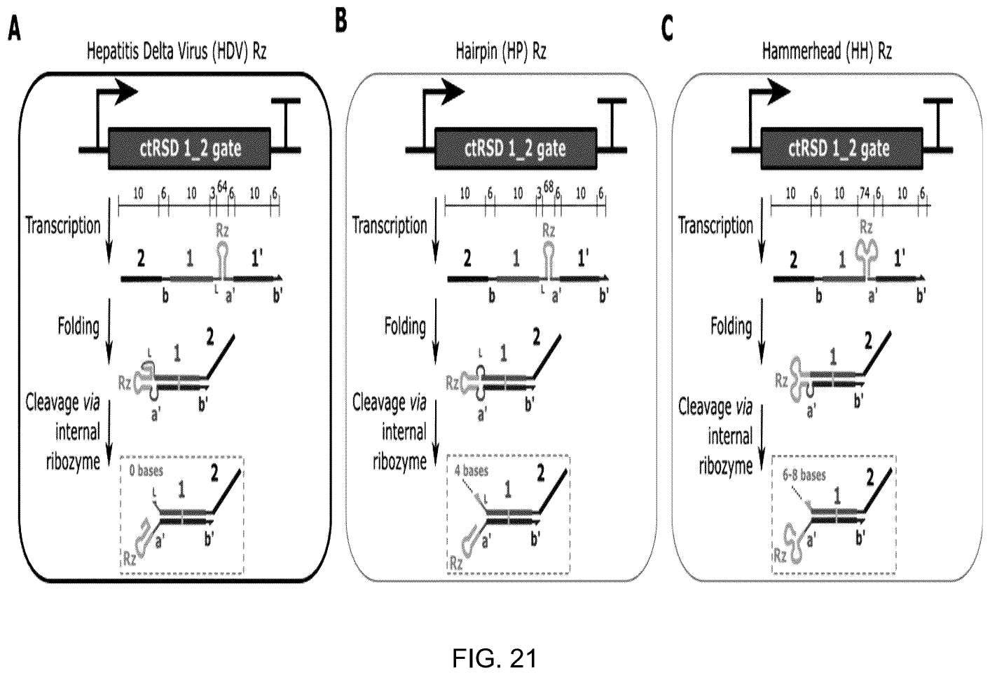

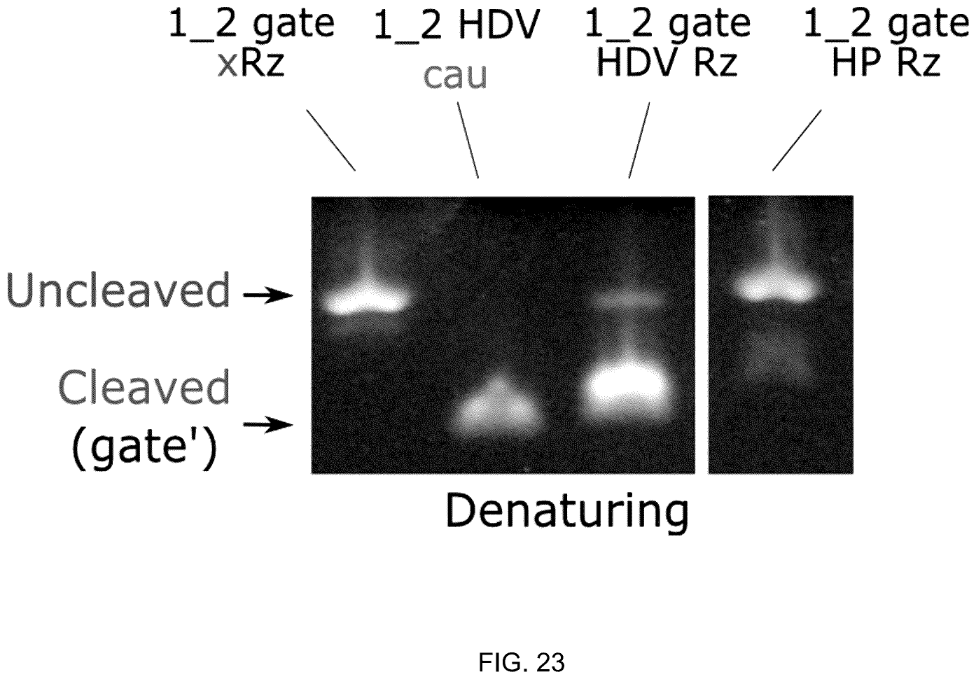

Three well characterized ribozymes were considered: the hammerhead ribozyme, the hairpin ribozyme, and the hepatitis delta virus (HDV) ribozyme. The HDV ribozyme has several advantages over the hammerhead and hairpin ribozymes. First, the HDV ribozyme folds quickly into a stable structure, likely making it resistant to misfolding across different flanking sequences. Second, the rate constant for HDV ribozyme cleavage has been reported as 52 min⁻¹ in certain settings, compared to 1 min⁻¹ for the hammerhead or 0.5 min⁻¹ to 0.05 min⁻¹ for the hairpin ribozymes. Lastly, the HDV ribozyme has little sequence preference upstream of the cleavage site. Both the hammerhead and hairpin ribozymes have cleavage site sequence constraints and their cleavage sites are flanked by RNA duplexes thus requiring a dissociation step following cleavage to separate the two strands. This dissociation step is particularly problematic in our ctRSD gate designs, in which the ssRNA toehold for strand displacement must be exposed after cleavage. In our designs, the hammerhead and hairpin ribozymes require 6 and 4 bases, respectively, to dissociate after cleavage to expose the toehold for strand displacement (FIG. 21). In the case of the hammerhead ribozyme, these 6 bases are likely to remain hybridized most of the time after cleavage, impeding RNA strand displacement. The HDV ribozyme does not suffer from these sequence limitations, driving this choice for our designs. We found the HDV ribozyme resulted in the desired efficient and rapid cleavage in our RNA gates (FIG. 6 and FIG. 22). We also tested a ctRSD gate with the hairpin ribozyme, but much less cleavage was observed than with the HDV ribozyme (FIG. 23).

Equilibrium Analysis with NUPACK

NUPACK 3.2.2 was used for equilibrium analysis of RNA complexes. We used the default NUPACK parameters for RNA (1.0 mol/L Na+ and 0 mol/L Mg++, dangles: some). Although there is 6 mmol/L MgCl₂ in our transcription buffer, there is a total of 8 mmol/L NTPs, which will sequester MgCl₂, so the concentration of free Mg++ is unknown. For RNA analysis, the default salt conditions are the only options. Unless otherwise state, analysis was conducted at 37° C. with 1 μmol/L of each RNA species. Changing the equimolar concentration of the RNA species between 10 nmol/L and 100 μmol/L does not change the predicted equilibrium concentrations.

For analysis of the reaction I1+1_2 gate↔I1:gate′+O2 the strands supplied to NUPACK are shown below:

I1
(SEQ ID NO.: 55)
5′GGGAGAUUCGUCUCCCAUCACUUCACAACAUCAAUAUAACCCCUUG
GGGCCUCUAAACGGGUCUUGAGGGGUUUUUUG
1_2 strand(O2):
(SEQ ID NO.: 56)
5′GGGAGAUUCGUCUCCCACCACAUACUAAUUAACUUACUUCACAUUC
1_2 gate′:
(SEQ ID NO.: 57)
5′GGGUCGGCAUGGCAUCUCCACCUCCUCGCGGUCCGACCUGGGCUAC
UUCGGUAGGCUAAGGGAGUGAUGUUGUGAAGUGAGUUAAUCUAUAAC
CCCUUGGGGCCUCUAAACGGGUCUUGAGGGGUUUUUUG

The 1_2 gate′ sequence contains the HDV ribozyme sequence. However, the HDV ribozyme structure is a pseudoknot, which NUPACK is incapable of predicting. Thus, the secondary structure of the HDV ribozyme in NUPACK does not represent its real structure. We found that the first two 5′ bases of the T7 RNAP terminator sequence n 11 (5′ CU) o were predicted to hybridize to part of the HDV ribozyme sequence on the 1_2 gate. However, this region of the ribozyme sequence is expected to be double stranded in the true ribozyme structure. To remove the influence of these spurious bases from the equilibrium analysis in NUPACK, the first C of the T7 RNAP terminator sequence was changed to an A (highlighted in yellow in the sequence above). This was done for all input sequences when analyzing these sequences in NUPACK.

Modeling ctRSD Circuit Reactions

### Model Assumptions and Reactions

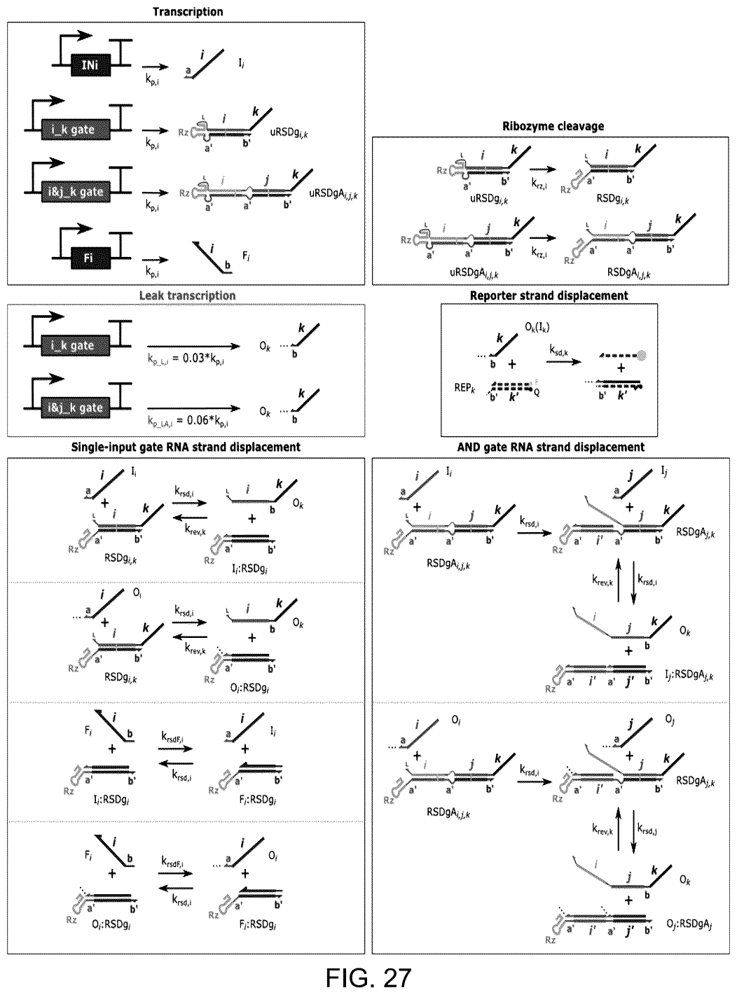

RNA strand displacement reactions were modeled using ordinary differential equations derived from mass action kinetics. All modeled reactions are shown in FIG. 27. In our model transcription was simplified to a first order process, whereby transcription rate is linearly proportional to the template concentration (kₚ*[template]). This assumption ignores transcriptional loading effects that arise when the concentration of polymerase is not in excess of the total concentration of total transcription templates. Thus, in our model, the first order transcription rate constant (kₚ) depends both on the concentration of T7 RNAP and the total concentration of templates, i.e. transcriptional load (FIG. 41). To account for these dependencies, we used an experimental calibration sample to obtain kₚ values for a given T7 RNAP concentration and total template concentration. The kₚ value obtained from this calibration sample was then used to simulate experimental samples with the same conditions (FIG. 42). Because all of the transcripts in this study possess the same 5′ sequence, we assumed kₚ was the same for all transcripts. Because co-transcriptional folding is 10-fold faster than transcription, we assume that the gates fold instantaneously upon transcription, unless otherwise stated.

The leak reaction in the ctRSD system was modeled by assuming that a small fraction of each ctRSD gate produced is as reactive as the designed output of the gate. Thus, a leak term was introduced in which an output is directly produced from its ctRSD gate template (k_{pL}*[ctRSD gate template]). k_{pL} is the first order leak transcription rate constant. For single input ctRSD gates, we found a k_{pL} that was 3% of kₚ recapitulated our experimental observations. This 3% leak transcription was used for all single input gates. We found that a 3% transcriptional leak for AND gates resulted in less leak than we observed in experiments. We reasoned this might be because each AND gate possesses two dsRNA domains. If we assume that each dsRNA stem has a 97% chance of being transcribed and folded correctly, we expect the chances an AND gate is correctly produced to be (0.97)²=94.1%. Based on this analysis, we assume a 6% transcriptional leak (k_{pLA}) for all AND gates in the study. We also assumed the reactions between AND gates and their first inputs were irreversible because the reverse reaction is facilitated by a one base toehold. The reverse reaction between an AND gate and its final output was included in the model.

Beyond the leak reaction described above, our model ignores other potential side reactions that are not expected to significantly influence dynamics. First, any gate possessing an output complementary to another gate could react via a 0 base strand displacement mechanism. This reaction was not included in the model because it occurs two to three orders of magnitude slower than the designed RNA strand displacement reactions. Second, an input can react with an RNA strand displacement gate prior to ribozyme cleavage. However, a mutant ctRSD gate that could not cleave reacted much slower with input than the self-cleaving ctRSD gate (FIG. 28). We assumed this side reaction would not greatly influence the observed kinetics at the low concentrations expected for the uncleaved ctRSD gate.

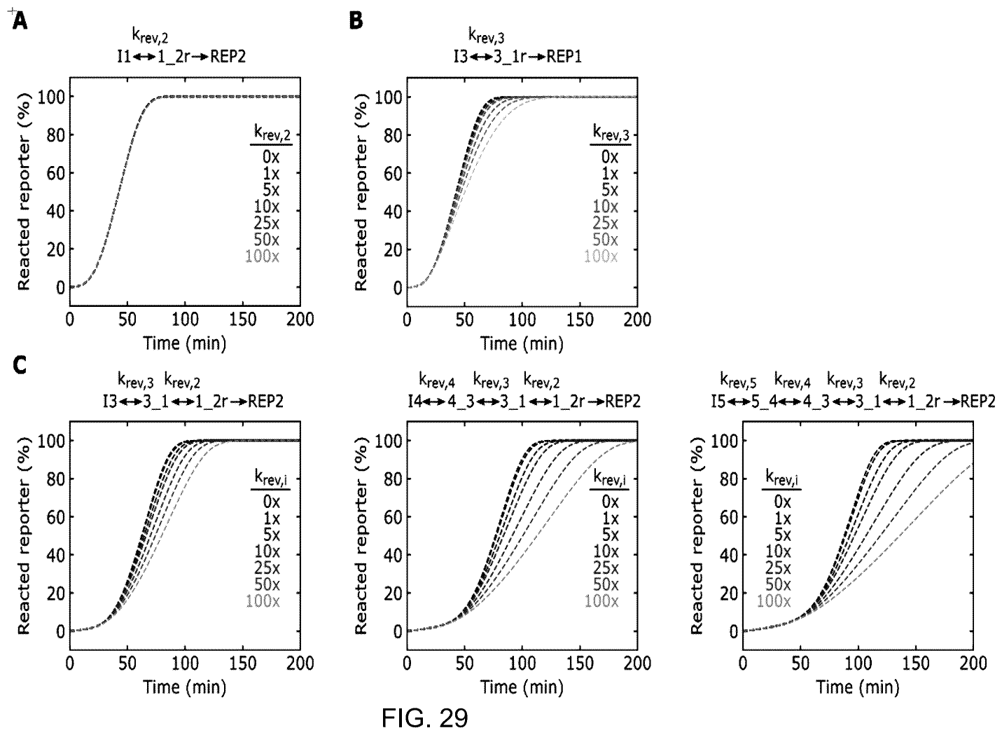

The model implementation pools output strands from gates with different input domains. For example, if both a 4_1 and 5_1 gate are present in a simulation the model only tracks the total O1 produced and does not explicitly track O1 released from the 4_1 gate and the O1 released from the 5_1 gate (FIG. 27). In most of our simulations, this issue does not arise as there is only a single gate releasing a given output. However, for circuits with OR elements, the reverse reaction for each gate will be overestimated. For example, for a 4_1 and 5_1 OR element the reverse rate for the 4_1 gate is kᵣₑᵥ*[14:RSDg4]*[O1], but O1 can come from both the 4_1 and the 51 gate, and only O1 from the 4_1 gate can participate in this reverse reaction. In each OR gate experiment, the input template concentrations were equal and the gate template concentrations were equal. Thus, the overcounting of outputs would only change the reverse reaction rate 2-fold. For single-layer OR gates, the largest circuits simulated in which two gates produced the same output, a significant change in kinetics is not observed until the reverse rate constants are increased 25-fold (FIGS. 29, A and B). Based on these results, the overcounting in OR gate reverse reaction rates should not influence the results for the networks simulated. A more rigorous model that tracks which gates the outputs come from could occur as ctRSD circuits expand.

Finally, the model does not consider any loss of T7 RNAP activity or depletion of NTPs during transcription. Thus, the model may become inaccurate when simulating experimental times>(4 to 5) h, as T7 RNAP activity will have decreased significantly. For slow reactions that are limited by transcription (i.e., transcription of leak products), the loss of T7 RNAP activity will eventually result in a plateau in output. The model will not capture this.

Kinetic Parameters Used to Model ctRSD Circuits

In toehold mediated DNA strand displacement (DSD), the rate of the strand displacement reaction is correlated to the binding energy of the toehold. As binding energy increases with increasing toehold length, the same trend between toehold length and strand displacement rate enhancement is predicted for RSD as for DSD. Because rate enhancement is related to toehold binding energy, toehold sequence can also greatly influence the observed rate. For example, a strong 6 base toehold with high G-C content can result in rate constants near 10⁶ L mol⁻¹ s⁻¹, while weaker 6 base toehold sequences can result in rate constants closer to 104 L mol⁻¹ s⁻¹. The toehold on the DNA reporter contains five A or T bases and a single G, making it a weak toehold. Thus, a rate constant of 104 L mol⁻¹ s⁻¹ was used to model the reaction between the reporter and the 1_2r strand (k_{sd}). All reporting reactions are considered to be irreversible.

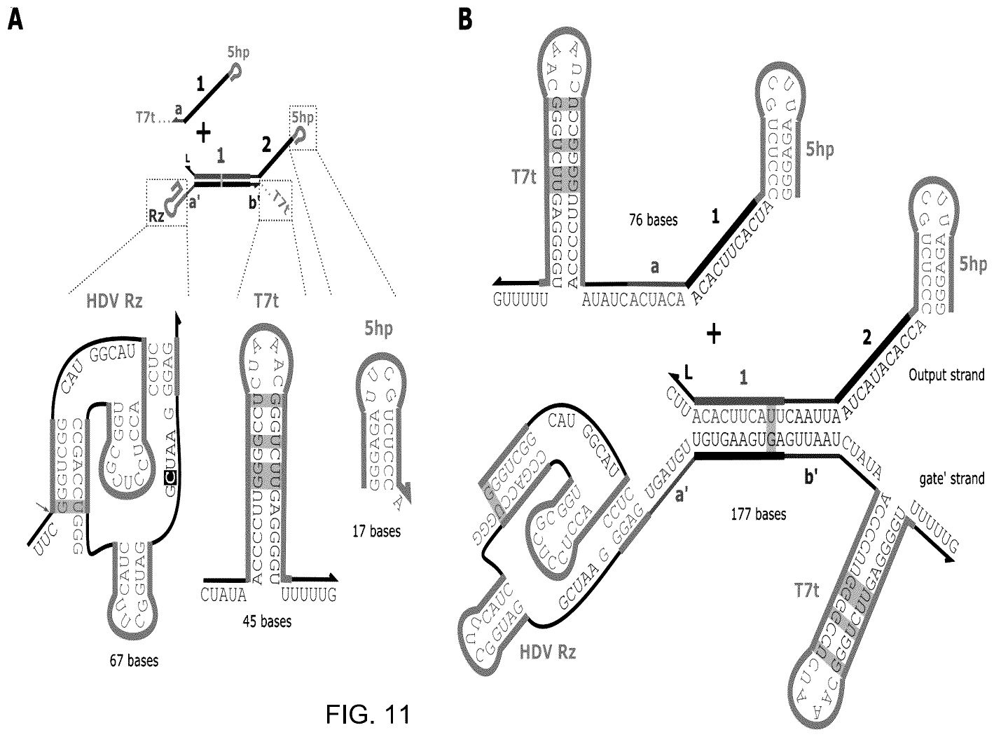

For the rate constant of the reaction between an input strand and its corresponding ctRSD gate complex (k_{rsd}), we found a value of 10³ L mol⁻¹ s⁻¹ best recapitulated our experimental results. This value is at least two orders of magnitude lower than expected for a 6 base toehold with moderate GC content. There is some evidence that RSD reaction rate constants can be an order of magnitude lower than DSD reaction rates for short toeholds. Additionally, the presence of the bulky HDV ribozyme structure directly upstream of the toehold on the ctRSD gate could lower the observed reaction rate (FIG. 11). This bulky structure could sterically clash with the terminator hairpin at the 3′ end of the input strand and effectively decrease the strand displacement rate. In support of this hypothesis, we found the introduction of a 4 base single-stranded spacer between the HDV ribozyme motif, and the toehold increased k_{rsd} to ≥10⁵ L mol⁻¹ s⁻¹. We assumed the k_{rsd} value was the same for all RSD reactions in ctRSD circuits, including for both toeholds of the AND gates. For the reaction between a fuel species and an input:gate′ complex, we assumed the same reaction rate constant as between the input and the ctRSD gate.

All the RNA strand displacement reactions in this study are reversible (FIG. 13). Estimation of the reverse reaction rate constants based on toehold length and sequence alone is confounded by reverse reactions replacing a G-C pair with a G-U wobble in the first two to three bases of branch migration (FIG. 13). This will reduce the rate of strand displacement, but the amount of this reduction is unknown. In DNA strand displacement, introduction of a mismatch at a similar position during branch migration can reduce the reaction rate by two to three orders of magnitude; presumably a G-U wobble would have a slightly less pronounced effect. Because we did not find an estimate for a comparable system in the literature, we estimated the reverse reaction rate constants from an equilibrium analysis of the strand displacement products. We used NUPACK 3.2.2 to calculate the equilibrium constant (Keq) for each complementary gate and input sequence. The reverse reaction rate constant (kᵣₑᵥ) was determined from the equilibrium constant as kᵣₑᵥ=k_{rsd}/Keq. Based on this analysis, we found gates with outputs possessing the b-toehold have reverse rate constants nearly three orders of magnitude lower than the forward rate constants. Gates with outputs possessing the a-toehold have reverse reaction rate constants only one order of magnitude lower than the forward rates (TABLE 2). To simplify the model, we assumed a single reverse rate constant for gates with b-toeholds (5 L mol⁻¹ s⁻¹) and for gates with a-toeholds (270 L mol⁻¹ s⁻¹). The reaction rate constants must be 10-fold larger than these values to begin to influence model predictions (FIG. 29).

The HDV ribozyme cleavage rate constant was estimated as 0.25 min⁻¹ (FIG. 22), consistent with previously reported in vitro values.

TABLE 2 lists an equilibrium analysis of RSD reactions across different ctRSD gates and inputs. All kinetic ate constants used in simulations are listed in TABLE 3, wherein the last two rate constants are used to model the leak transcription reaction for single input gates (kpL) and AND gates (kpLA).

TABLE 2

| Gate | I + gate ↔ I:gate + Out (Keq) | kᵣₑᵥ (L mol⁻¹ s⁻¹) |
|---|---|---|
| 1_2 gate | (0.93)²/(0.07)² = 176.51 | 5.67 |
| 3_2 gate | (0.905)²/(0.095)² = 90.75 | 11.01 |
| 4_2 gate | (0.942)²/(0.058)² = 263.78 | 3.79 |
| 5_2 gate | (0.939)²/(0.061)² = 236.36 | 4.22 |
| 1_4 gate | (0.65)²/(0.35)² = 3.45 | 289.86 |
| 3_4 gate | (0.62)²/(0.38)² = 2.66 | 375.94 |
| 4_1 gate | (0.72)²/(0.28)² = 6.61 | 151.29 |
| 5_1 gate | (0.66)²/(0.34)² = 3.77 | 265.25 |

TABLE 3

| Parameter | Value | Source |
|---|---|---|
| kₚ (s⁻¹) | 0.0075 to 0.019 | Calibrated for transcriptional load |
| k_{rz} (s⁻¹) | 4.167 × 10⁻³ | FIG. 22 |
| k_{sd} (L mol⁻¹ s⁻¹) | 1 × 10⁴ | EXAMPLE |
| k_{rsd} (L mol⁻¹ s⁻¹) | 1 × 10³ | EXAMPLE |
| k_{rsdF} (L mol⁻¹ s⁻¹) | 1 × 10³ | EXAMPLE |
| k_{rev,a} (L mol⁻¹ s⁻¹) | 2.7 ×10² | TABLE 2 |
| k_{rev,b} (L mol-1 s⁻¹) | 5 | TABLE 2 |
| k_{pL} (s⁻¹) | 3% of kₚ | Calibrated to data |
| k_{pLA} (s⁻¹) | 6% of kₚ | Calibrated to data |

### Modeling and Characterizing Leak

In the experiments, we observed a leak in which transcription of the 1_2r gate template in the absence of the I1 template resulted in a slow increase in DNA reporter signal. This leak reaction increased with increasing concentrations of T7 RNAP, i.e., the leak increased with increasing transcription rate (FIG. 30). The initial model of ctRSD circuits did not include terms capable of producing this leak (FIGS. 31, A and B). To include this observed leak in the model, we investigated three potential models of leak the pathway: Models 1, 2, and 3 in FIG. 31C. Both Model 2 and Model 3 can recapitulate the experimental data, but only Model 3 is consistent with the experimental results presented in FIG. 31. Unless otherwise stated, all other simulations included the leak reactions depicted in Model 3.

Two additional models for leak were eliminated. (1) Short transcripts produced during abortive cycling by T7 RNAP could include part of the output domains and react with the DNA reporter. This model was considered unlikely because short abortive transcripts typically range from (2 to 12) nucleotides but the gate transcripts possess a 17 nucleotide hairpin sequence at their 5′ end. Thus, any short transcripts produced during abortive cycling should not contain sequence complementarity with the reporter. (2) The ribozyme rapidly cleaves during transcription and releases the output before the bottom strand of the gate (gate′) is produced. The output strand could then irreversibly react with the DNA reporter before hybridizing to form a dsRNA gate. This model was considered unlikely because we measured the HDV ribozyme cleavage rate constant to be ˜0.25 per min (˜0.004 per s) in our assay conditions (FIG. 22). This value is consistent with previously published values for HDV ribozyme self-cleavage in vitro. From our simulation results, the transcription rate constant in our experiments was ˜0.01 per s, indicating transcription proceeds much faster than ribozyme cleavage. The results in FIG. 32 are also inconsistent with this model of leak because the leak is still observed even when the RNA gate is transcribed in isolation.

(D) Simulation results (dashed lines) for Model 1 compared to experimental results (solid lines). In the simulations, a kₗₑₐₖ of 15 L mol⁻¹ s⁻¹ was used for the 0 base toehold reaction between the 1_2r gate complex and the DNA reporter. This is an order of magnitude higher than reported previously. (E) Simulation results (dashed lines) for Model 2 compared to experimental results (solid lines). In the simulations, a k_{fold} of 0.15 s⁻¹ was used. Considering that co-transcriptional folding occurs much faster than transcription, the k_{fold} parameter may be taken as the time required to produce the transcript, during which the nascent transcript could react with the DNA reporter. A k_{fold} of 0.15 s⁻¹ corresponds to a transcript produced every 6.67 s, and this corresponds to the transcription rate of ˜27 nt/s for the 183 nt 1_2r gate transcript. This transcription rate is within a factor of 1.5 of previously reported transcription rates for T7 RNAP, supporting the feasibility of the k_{fold} parameter that recapitulates the experimental data. (F) Simulation results (dashed lines) for Model 3 compared to experimental results (solid lines). In the simulations, a production rate of truncated 1_2r gate products (kₚ,L) that was 3% of the production rate of correct products (kₚ) was used. The reaction between the DNA reporter and the truncated 1_2r gate product was assumed to have the same rate constant (k_{sd}) as the reaction between the DNA reporter and the 1_2r strand. All other rate constants are in TABLE 3. The experimental results are also presented in FIG. 7.

### Analysis of Deviations Between Experiments and Simulations

Across our experiments there were two minor deviations from simulation predictions. Deviation 1: There was lower leak than predicted between ctRSD gates, which could be the result of steric hindrance between leak products and gates (FIG. 36). Deviation 2: The gates that take 14 as an input reacted slower than the gates that take other inputs. The 4_2r gate was noticably slower than the three other single input gates tested (FIG. 8A), and the 41 gate was slower than the 5_1 gate in a two-layer cascade (FIG. 37B). The 4_1 gate was also slower in a logic cascade than the 5_1 gate (FIG. 37D). We hypothesized that the strand displacement rate constant for gates that take 14 as an input could be lower than the other domains due to the high UA content at the start of branch migration (FIG. 13). Similar sequences have been shown to significantly decrease overall strand displacement kinetics. Decreasing k_{rsd} 4-fold in our simulations for just the gates that take I4 as an input resulted in better model agreement across experiments (FIG. 37).

1_2r Gates with Different Toehold Lengths

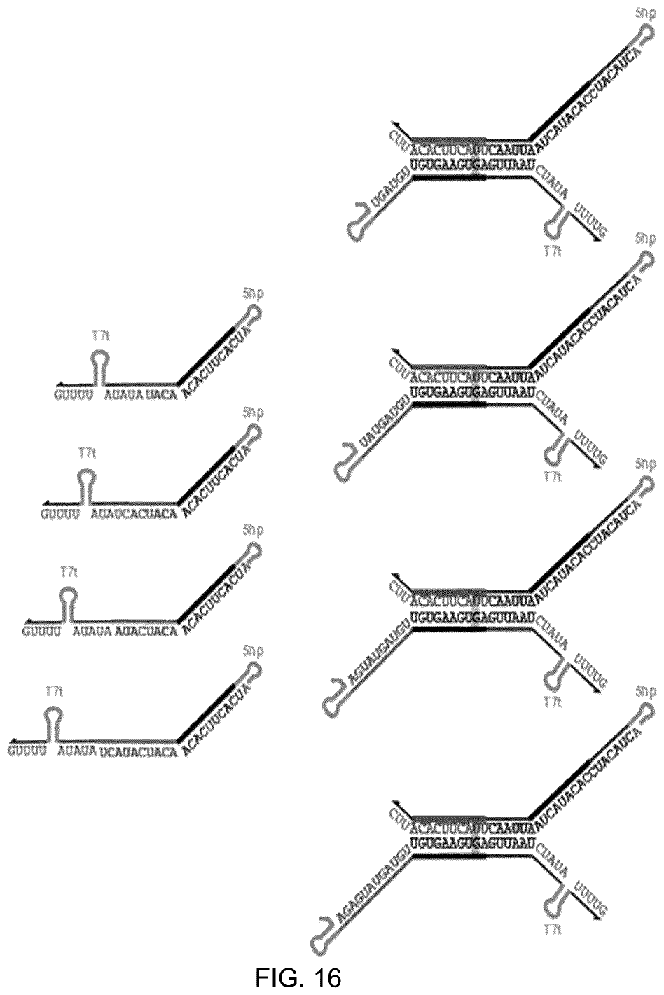

The kinetics of toehold-mediated strand displacement reactions can be controlled by toehold length. Here, we explore how toehold length influenced the kinetics of ctRSD circuit reactions. The initial design for the 1_2r gate included a 6 base single-stranded input toehold, which we would expect to result in a rate constant near the maximum theoretical limit (10⁶ L mol⁻¹ s⁻¹). However, our simulations indicated that the forward strand displacement rate constant between the 1_2r gate and 11 was only 10³ L mol⁻¹ s⁻¹. We theorized steric hindrance between the ribozyme and the input strand could result in slower strand displacement because the 6 base toehold is directly adjacent to the bulky HDV ribozyme motif (FIG. 11). Thus, in addition exploring the influence of toehold length on kinetics, we also explored the influence of including a single-stranded spacer sequence between the ribozyme motif and the toehold. To do this, we designed 1_2r gates with (6, 8, 10, and 12) base toeholds and 11 variants possessing (4, 6, 8, or 10) base toeholds and combinatorically tested all gate and input combinations. Schematics with sequences are presented in FIG. 16. We first confirmed increasing toehold length did not influence gate folding and/or cleavage. FIG. 38 demonstrates that the 1_2r gate toehold variants cleaved as designed. FIG. 39 shows that increasing toehold length did not increase leak with the DNA reporter, suggesting proper folding.

We next evaluated RSD kinetics for all gate and input toehold length combinations. These experiments encompassed toehold lengths of 4 bases to 10 bases with spacer lengths varying from (0 to 8) bases depending on the input toehold length (FIGS. 40, A and B). In these experiments, we were not able to resolve reaction rate constants greater than 10⁵ L mol⁻¹ s⁻¹ (FIG. 40C). When the strand displacement reaction rate gets this high, the overall rate of output release becomes limited by transcription and gate cleavage, rather than strand displacement. Thus, we report all reaction rate constants near this 10⁵ L mol⁻¹ s⁻¹ limit as ≥10⁵ L mol⁻¹ s⁻¹ (FIG. 40B).

FIG. 40D show the kinetic traces for each gate and input toehold combination, highlighting the influence of spacer length on reaction kinetics for each input toehold length. Inclusion of a spacer generally increases reaction rate, but the spacer length that saturates the reaction rate decreases as input toehold length increases. An explanation for this observation could be: the weaker the input binding energy, the greater the influence of steric hindrance on the reaction rate. For example, the input with a 4 base toehold binds weakly to the 1_2r gate toehold, so a long spacer is required to completely remove any effect of steric hindrance. Conversely, for the input with a 10 base toehold, the same kinetics are observed for a 0 base and 2 base spacer. In this case, the input with the 10 base toehold can be viewed as an input with a 6 base toehold binding to a gate with a 4 base spacer, or an input with an 8 base toehold binding to a gate with a 2 base spacer. Both those reactions occur at a rate near the maximum value. Put another way, once the input toehold is long enough, increasing the length of the a-toehold (input) and a′-toehold (gate) together has almost the same effect as simply increasing the spacer length, i.e. increasing the a′-toehold (gate) without increasing the a-toehold (input). In support of this hypothesis, the 8 base a-toehold (input) and 8 base a′-toehold (gate) reaction rate constant is close to the 6 base a-toehold (input) and 8 base a′-toehold (gate) reaction rate constant (FIG. 40B).

FIG. 40E shows the kinetic traces for each gate and input toehold combination, highlighting the influence of toehold length on reaction kinetics for each spacer length. With the exception of the input with a 4 base a-toehold, most of the changes in kinetics observed across toehold length can be attributed to the increase in a′-toehold (spacer) length. For example, with long enough spacers, inputs with (6, 8, and 10) base toeholds exhibit strand displacement constants close to the maximum value (≥10⁵ L mol⁻¹ s⁻¹).

For traditional DNA and RNA strand displacement, in which double-stranded complexes are pre-annealed and gate toeholds have no secondary structure upstream, toeholds ≥6 bases should result in reaction rate constants at the theoretical maximum of ≈10⁶ L mol⁻¹ s⁻¹. We found similar results for ctRSD circuits when using a long enough spacer between the HDV ribozyme and the toehold. Regarding the input with a 4 base a-toehold, the reaction between this input and any of the 1_2r gates has a much lower thermodynamic driving force than the other input toeholds tested. This is because the 1_2r gates all possess a 6 base reverse toehold, i.e. completion of the forward strand displacement reaction results in a net loss of two base pairs compared to the intact 1_2r gate. The rate constant for a DNA strand displacement reaction between an input with a 4 base toehold and a gate with a 6 base reverse toehold (b-toehold) was measured to be between (10² and 10³) L mol⁻¹ s⁻¹. This aligns with our estimated rate constant of 2×10² L mol⁻¹ s⁻¹ for between the 4 base input toehold variant and ctRSD gates with either a 6 base or 8 base spacers (FIG. 40B). Together, these results suggest that ctRSD circuits should possess the same kinetic control of traditional toehold-mediated strand displacement, provided appropriate spacers are used.

Steric hindrance introduced by the ribozyme could also be used as an additional feature to tune strand displacement rates. Changing the spacer length adjacent to the ribozyme allows different strand displacement rate constants to be obtained, without needing to change the input's toehold length. For the 6 base a-toehold, varying spacer length changed the strand displacement rate constant by two orders of magnitude.

Potential Advantages of ctRSD Circuits Compared to DNA-Based Circuits

Should ctRSD circuits continue to prove as predictable and programmable as DNA-based circuits, ctRSD could serve as a more versatile alternative to DNA computing. Such a shift could be justified given the high fidelity and decreasing price of gene synthesis. Integrated DNA Technologies currently reports ≈80% of 30 base DNA oligonucleotides are the correct product compared to ≈100% for gBlocks of >125 bases. The low fidelity of DNA oligonucleotide synthesis requires the strands to be purified with gel electrophoresis and many DNA computing papers report the purification of individual dsDNA circuit complexes to obtain desired circuit function. For ctRSD circuits the high-fidelity gBlock synthesis is followed by a high-fidelity PCR step (<0.25% error) and high-fidelity transcription-T7 RNAP's nucleotide substitution rate is less than 1 in 17,000 bases. Further, encoding the dsRNA complex as a single transcript ensures the proper stoichiometry between the two gate strands, reducing leak pathways. Thus, ctRSD circuits remove the need for purification of circuit components before operation, greatly simplifying the workflow. Further, the per nanomole cost of a ctRSD gate template can be reduced to nearly that of analogous DNA gates with a few modifications to the protocol here.

Another advantage of using transcriptionally encoded circuits over DNA strand displacement circuits is the long-term stability of long linear DNA templates and DNA plasmids. For example, in many biosensor and diagnostic applications, circuit components are freeze dried for long-term storage and ease of transportation. These freeze-dried circuits are then activated by adding a liquid sample at the point of need. Both linear DNA templates on the order of 300 bases and DNA plasmids have been shown to remain stable for months after freeze drying. Short DNA strand displacement duplexes show significant decrease in performance only one week after freeze drying.

### Experimental Conditions, Transcription Rate Calibration, and Experimental Variability

TABLE 4 lists transcription template and T7 RNAP concentrations used in DNA reporter assays. In our experiments, the transcription rate was dependent on the total transcription template and T7 RNAP concentrations (FIG. 41). To ensure the same transcription load across different samples for the same ctRSD element or circuit, a template producing an unreactive input (Io) was added so that all samples had the same total template concentration. The total concentration of templates for each experiment is also presented in TABLE 4. Because the transcription rate differed across many experiments, the first order rate constant (kₚ) used to model transcription had to be calibrated for a given T7 RNAP and total template concentration (FIG. 42). The first order rate constant (kₚ) calibrated for each experiment is presented in TABLE 4. This transcription rate calibration should also calibrate for batch to batch variation in T7 RNAP stocks. Other than the differences arising from different experimental conditions or T7 RNAP batches, replicate measurements of circuit kinetics varied between 2% to 5%. TABLE 4 lists transcription template and T7 RNAP concentrations used in DNA reporter experiments. 500 nmol/L of DNA reporter was used in each experiment. For experiments in which multiple input template concentrations or input template combinations were tested, each column of values represents the nominal concentrations of each input template for each experiment.

TABLE 4

| FIG. 7D (I1 titration) | Values |
|---|---|
| [1_2r gate] (nmol/L) | 25 |
| [I1] (nmol/L) | 0, 2, 5, 5, 12.5, 25, 50 |
| [Io] (nmol/L) | 50, 47.5, 45, 37.5, 25, 0 |
| [T7 RNAP] (U/μL) | 1.0 |
| Total [templates] (nmol/L) | 75 |
| kₚ (s⁻¹) | 0.013 |
| FIG. 8A (orthogonal sequences) | Values |
| [Gates] (nmol/L) | 25 |
| [Ii] (nmol/L) | 0, 50 |
| [Io] (nmol/L) | 50, 0 |
| [T7 RNAP] (U/μL) | 1.0 |
| Total [templates] (nmol/L) | 75 |
| kₚ (s⁻¹) | 0.013 |
| FIG. 9C (1\|3_2r) | Values |
| [1_2r gate] (nmol/L) | 25 |
| [3_2r gate] (nmol/L) | 25 |
| [I1] (nmol/L) | 0, 50, 0, 50 |
| [I3] (nmol/L) | 0, 0, 50, 50 |
| [Io] (nmol/L) | 100, 50, 50, 0 |
| [T7 RNAP] (U/μL) | 1.2 |
| Total [templates] (nmol/L) | 150 |
| kₚ (s⁻¹) | 0.008 |
| FIG. 9F (3&1_2r) | Values |
| [3&1_2r gate] (nmol/L) | 25 |
| [I1] (nmol/L) | 0, 50, 0, 50 |
| [I3] (nmol/L) | 0, 0, 50, 50 |
| [Io] (nmol/L) | 100, 50, 50, 0 |
| [T7 RNAP] (U/μL) | 1.0 |
| Total [template] (nmol/L) | 125 |
| kₚ (s⁻¹) | 0.009 |
| FIG. 9I (Catalytic Amp) | Values |
| [1_2r gate] (nmol/L) | 25 |
| [I1] (nmol/L) | 1.25, 2.50, 1.25, 2.50 |
| [F1] (nmol/L) | 0, 0, 25, 25 |
| [Io] (nmol/L) | 26.25, 25, 1.25, 0 |
| [T7 RNAP] (U/μL) | 1.2 |
| Total [template] (nmol/L) | 52.5 |
| kₚ (s⁻¹) | 0.010 |
| FIG. 10B (4-layer cascade) | Values |
| [1_2r gate] (nmol/L) | 25, 25, 25, 25, 25, 25, 25, 25 |
| [5_1 gate] (nmol/L) | 0, 25, 25, 25, 0, 25, 25, 25 |
| [4_5 gate] (nmol/L) | 0, 0, 25, 25, 0, 0, 25, 25 |
| [3_4 gate] (nmol/L) | 0, 0, 0, 25, 0, 0, 0, 25 |
| [I1] (nmol/L) | 0, 0, 0, 0, 50, 0, 0, 0 |
| [I3] (nmol/L) | 0, 0, 0, 0, 0, 0, 0, 50 |
| [I4] (nmol/L) | 0, 0, 0, 0, 0, 0, 50, 0 |
| [I5] (nmol/L) | 0, 0, 0, 0, 0, 50, 0, 0 |
| [Io] (nmol/L) | 125, 100, 75, 50, 75, 50, 25, 0 |
| [T7 RNAP] (U/μL) | 1.5 |
| Total [template] (nmol/L) | 150 |
| kₚ (s⁻¹) | 0.0075 |
| FIG. 10C (1\|3\|4\|5_2r) | Values |
| [1_2r gate] (nmol/L) | 12.5 |
| [3_2r gate] (nmol/L) | 12.5 |
| [4_2r gate] (nmol/L) | 12.5 |
| [5_2r gate] (nmol/L) | 12.5 |
| [11] (nmol/L) | 0, 50, 0, 0, 0 |
| [13] (nmol/L) | 0, 0, 50, 0, 0 |
| [14] (nmol/L) | 0, 0, 0, 50, 0 |
| [15] (nmol/L) | 0, 0, 0, 0, 50 |
| [lo] (nmol/L) | 50, 0, 0, 0, 0 h |
| [T7 RNAP] (U/μL) | 1.2 |
| Total [template] (nmol/L) | 100 |
| kₚ (s⁻¹) | 0.015 |
| FIG. 10D (5&4_1 to 3&1_2r) | Values |
| [5&4_2r gate] (nmol/L) | 25 |
| [3&1_2r gate] (nmol/L) | 25 |
| [13] (nmol/L) | 0, 25, 0, 0, 25, 25, 0, 25 |
| [14] (nmol/L) | 0, 0, 25, 0, 25, 0, 25, 25 |
| [15] (nmol/L) | 0, 0, 0, 25, 0, 25, 25, 25 |
| [lo] (nmol/L) | 75, 50, 50, 50, 25, 25, 25, 0 |
| [T7 RNAP] (U/μL) | 1.2 |
| Total [template] (nmol/L) | 125 |
| kₚ (s⁻¹) | 0.0075 |
| FIG. 10E (4\|5_1 to 3&1_2r) | Values |
| [4_1 gate] (nmol/L) | 25 |
| [5_1 gate] (nmol/L) | 25 |
| [3&1_2r gate] (nmol/L) | 25 |
| [I3] (nmol/L) | 0, 25, 0, 0, 25, 25, 0, 25 |
| [I4] (nmol/L) | 0, 0, 25, 0, 25, 0, 25, 25 |
| [I5] (nmol/L) | 0, 0, 0, 25, 0, 25, 25, 25 |
| [Io] (nmol/L) | 75, 50, 50, 50, 25, 25, 25, 0 |
| [T7 RNAP] (U/μL) | 1.5 |
| Total [template] (nmol/L) | 150 |
| kₚ (s⁻¹) | 0.010 |
| FIG. 10F (5&4_1 to 1\|3_2r) | Values |
| [5&4_2r gate] (nmol/L) | 25 |
| [1_2r gate] (nmol/L) | 25 |
| [3_2r gate] (nmol/L) | 25 |
| [I3] (nmol/L) | 0, 25, 0, 0, 25, 25, 0, 25 |
| [I4] (nmol/L) | 0, 0, 25, 0, 25, 0, 25, 25 |
| [I5] (nmol/L) | 0, 0, 0, 25, 0, 25, 25, 25 |
| [Io] (nmol/L) | 75, 50, 50, 50, 25, 25, 25, 0 |
| [T7 RNAP] (U/μL) | 1.5 |
| Total [template] (nmol/L) | 150 |
| kₚ (s⁻¹) | 0.010 |

The following are incorporated by reference in their entirety.
J. K. Jung, K. K. Alam, M. S. Verosloff, D. A. Capdevila, M. Desmau, P. R. Clauer, J. W. Lee, P. Q. Nguyen, P. A. Pastén, S. J. Matiasek, J.-F. Gaillard, D. P. Giedroc, J. J. Collins, J. B. Lucks, Cell-free biosensors for rapid detection of water contaminants. Nature Biotechnology. 38, 1451-1459 (2020).
J. K. Jung, K. K. Alam, J. B. Lucks, bioRxiv, in press, doi:10.1101/2021.03.16.435693.
K. Pardee, A. A. Green, M. K. Takahashi, D. Braff, G. Lambert, J. W. Lee, T. Ferrante, D. Ma, N. Donghia, M. Fan, N. M. Daringer, I. Bosch, D. M. Dudley, D. H. O'Connor, L. Gehrke, J. J. Collins, Rapid, Low-Cost Detection of Zika Virus Using Programmable Biomolecular Components. Cell. 165, 1255-1266 (2016).
M. K. Takahashi, X. Tan, A. J. Dy, D. Braff, R. T. Akana, Y. Furuta, N. Donghia, A. Ananthakrishnan, J. J. Collins, A low-cost paper-based synthetic biology platform for analyzing gut microbiota and host biomarkers. Nat. Commun. 9, 3347 (2018).
C. Zhang, Y. Zhao, X. Xu, R. Xu, H. Li, X. Teng, Y. Du, Y. Miao, H. Lin, D. Han, Cancer diagnosis with DNA molecular computation. Nature Nanotechnology. 15, 709-715 (2020).
L. Qian, E. Winfree, Scaling Up Digital Circuit Computation with DNA Strand Displacement Cascades. Science. 332, 1196 (2011).
Bae, W., Stan, G.-B. V. & Ouldridge, T. E. In situ Generation of RNA Complexes for Synthetic Molecular Strand-Displacement Circuits in Autonomous Systems. Nano Lett. 21, 265-271 (2021).
Bhadra, S. & Ellington, A. D. Design and application of cotranscriptional non-enzymatic RNA circuits and signal transducers. Nucleic Acids Research 42, e58-e58 (2014).

The processes and articles described herein may be embodied in, and fully automated via, software code modules executed by a computing system that includes one or more general purpose computers or processors. The code modules may be stored in any type of non-transitory computer-readable medium or other computer storage device. Some or all the methods may alternatively be embodied in specialized computer hardware. In addition, the components referred to herein may be implemented in hardware, software, firmware, or a combination thereof.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

Any logical blocks, modules, and algorithm elements described or used in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described or used in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix (s) as used herein is intended to include both the singular and the plural of the term that it modifies, thereby including at least one of that term (e.g., the colorant(s) includes at least one colorants). Option, optional, or optionally means that the subsequently described event or circumstance can or cannot occur, and that the description includes instances where the event occurs and instances where it does not. As used herein, combination is inclusive of blends, mixtures, alloys, reaction products, collection of elements, and the like.

As used herein, a combination thereof refers to a combination comprising at least one of the named constituents, components, compounds, or elements, optionally together with one or more of the same class of constituents, components, compounds, or elements.

All references are incorporated herein by reference.

The use of the terms “a,” “an,” and “the” and similar referents in the context of describing the invention (especially in the context of the following claims) are to be construed to cover both the singular and the plural, unless otherwise indicated herein or clearly contradicted by context. It can further be noted that the terms first, second, primary, secondary, and the like herein do not denote any order, quantity, or importance, but rather are used to distinguish one element from another. It will also be understood that, although the terms first, second, etc. are, in some instances, used herein to describe various elements, these elements should not be limited by these terms. For example, a first current could be termed a second current, and, similarly, a second current could be termed a first current, without departing from the scope of the various described embodiments. The first current and the second current are both currents, but they are not the same condition unless explicitly stated as such.

The modifier about used in connection with a quantity is inclusive of the stated value and has the meaning dictated by the context (e.g., it includes the degree of error associated with measurement of the particular quantity). The conjunction or is used to link objects of a list or alternatives and is not disjunctive; rather the elements can be used separately or can be combined together under appropriate circumstances.

### PARTS LIST

ctRSD gate 200
output strand 201
gate prime strand 202
hairpin-forming sequence 203
output branch migration domain 204
output toehold domain 205
input branch migration domain 206
output wobble domain 207
linker (L) sequence 208
self-cleaving ribozyme 209
input toehold domain 210
substrate domain 211
gate prime wobble domain 212
output toehold sequester domain 213
transcription termination sequence 214
input template strand 215
strand displacement product 216

What is claimed is:

1. A method for the production of an RNA toehold exchange gate, that method comprising:
encoding DNA for the production of an RNA toehold exchange gate; and
producing the RNA toehold exchange gate by transcription,
wherein the RNA toehold exchange gate is co-transcriptionally folded into a kinetically trapped intermediate following transcription.

2. A method for using co-transcriptional RNA strand displacement (ctRSD) circuits for the execution of programmable logic, amplification, or cascades, that method comprising:
encoding DNA for the production of an RNA toehold exchange gate;
producing the RNA toehold exchange gate by transcription; and
allowing the RNA toehold exchange gate to bind to a complementary single-stranded input,
wherein the RNA toehold exchange gate is co-transcriptionally folded into a kinetically trapped intermediate following transcription.

3. An RNA toehold exchange gate for use in co-transcriptional RNA strand displacement (ctRSD) circuits, the RNA toehold exchange gate comprising:
a 5′ hairpin;
a 3′ terminator;
a self-cleaving ribozyme; and
a gate output sequence limited to cystosine, adenine, or uracil bases.

4. A DNA sequence encoding an RNA toehold exchange gate for use in co-transcriptional RNA strand displacement (ctRSD) circuits, the DNA sequence comprising:
a 5′ hairpin:
(SEQ ID NO.: 1)
GGGAGATTCGTCTCCCA;
an HDV ribozyme:
(SEQ ID NO.: 2)
TTCGGGTCGGCATGGCATCTCCACCTCCTCGCGGTCCGACCTGG
GCTACTTCGGTAGGCTAAGGGAG;
one T7 terminator selected from the group consisting of:
(SEQ ID NO.: 4)
CTATAACCCCTTGGGGCCTCTAAACGGGTCTTGAGGGGTTTTTTG
and
(SEQ ID NO.: 5)
ATATAACCCCTTGGGGCCTCTAAACGGGTCTTGAGGGGTTTTTTG.

5. An ctRSD gate (200) for performing co-transcriptional encoding, the ctRSD gate (200) comprising:
an output strand (201) comprising:
an input branch migration domain (206);
an output branch migration domain (204) sequentially connected to the input branch migration domain (206); and
an output toehold domain (205) sequentially interposed between the input branch migration domain (206) and the output branch migration domain (204); and
a gate prime strand (202) electrostatically associated with the output strand (201) and comprising:
a self-cleaving ribozyme (209);
an output toehold sequester domain (213) sequentially connected to the self-cleaving ribozyme (209);
a substrate domain (211) sequentially interposed between the self-cleaving ribozyme (209) and the output toehold sequester domain (213), such that a portion of the substrate domain (211) is sequentially complementary to a portion of the input branch migration domain (206) that results in the gate prime strand (202) being electrostatically associated with the output strand (201); and
an input toehold domain (210) sequentially interposed between the self-cleaving ribozyme (209) and the substrate domain (211),wherein the output strand (201) and the gate prime strand (202) indepedently consist essentially of RNA.

6. The ctRSD gate (200) of claim 5, wherein the output strand (201) further comprises a hairpin-forming sequence (203) sequentially connected to the output branch migration domain (204) such that output branch migration domain (204) is sequentially interposed between the hairpin-forming sequence (203) and the output toehold domain (205).

7. The ctRSD gate (200) of claim 5, wherein the output strand (201) further comprises an output wobble domain (207) sequentially connected to the input branch migration domain (206) such that the output wobble domain (207) is sequentially interposed between a first portion of the input branch migration domain (206) and a second portion of the input branch migration domain (206).

8. The ctRSD gate (200) of claim 5, wherein the output strand (201) further comprises a linker sequence (208) sequentially connected to the input branch migration domain (206) such that input branch migration domain (206) is sequentially interposed between the linker sequence (208) and the linker sequence (208).

9. The ctRSD gate (200) of claim 5, wherein the gate prime strand (202) further comprises a transcription termination sequence (214) sequentially connected to the output toehold sequester domain (213) such that output toehold sequester domain (213) is sequentially interposed between the transcription termination sequence (214) and the substrate domain (211).

10. The ctRSD gate (200) of claim 5, wherein the gate prime strand (202) further comprises a gate prime wobble domain (212) sequentially connected to the substrate domain (211) such that the gate prime wobble domain (212) is sequentially interposed between a first portion of the substrate domain (211) and a second portion of the substrate domain (211).

11. The ctRSD gate (200) of claim 5, wherein the output strand (201) produces a strand displacement product (216) in response to contact with an input template strand (215).

12. The ctRSD gate (200) of claim 5, wherein the output strand (201) further comprises a second input branch migration domain (206).2 sequentially connected to the input branch migration domain (206).

13. The ctRSD gate (200) of claim 5, wherein the gate prime strand (202) further comprises a second substrate domain (211).2 sequentially connected to the substrate domain (211).

14. A process for producing a strand displacement product (216), the process comprising:
providing a ctRSD gate (200);
contacting the ctRSD gate (200) with a input template strand (215); and
producing the strand displacement product (216) from the ctRSD gate (200) in response to contacting the ctRSD gate (200) with the input template strand (215).

15. The process of claim 10, wherein the ctRSD gate (200) comprises:
an output strand (201) comprising:
an input branch migration domain (206);
an output branch migration domain (204) sequentially connected to the input branch migration domain (206); and
an output toehold domain (205) sequentially interposed between the input branch migration domain (206) and the output branch migration domain (204); and
a gate prime strand (202) electrostatically associated with the output strand (201) and comprising:
a self-cleaving ribozyme (209);
an output toehold sequester domain (213) sequentially connected to the self-cleaving ribozyme (209);
a substrate domain (211) sequentially interposed between the self-cleaving ribozyme (209) and the output toehold sequester domain (213), such that a portion of the substrate domain (211) is sequentially complementary to a portion of the input branch migration domain (206) that results in the gate prime strand (202) being electrostatically associated with the output strand (201); and
an input toehold domain (210) sequentially interposed between the self-cleaving ribozyme (209) and the substrate domain (211), wherein the output strand (201) and the gate prime strand (202) indepedently consist essentially of RNA.

16. The process of claim 11, wherein the output strand (201) further comprises a hairpin-forming sequence (203) sequentially connected to the output branch migration domain (204) such that output branch migration domain (204) is sequentially interposed between the hairpin-forming sequence (203) and the output toehold domain (205).

17. The process of claim 11, wherein the output strand (201) further comprises an output wobble domain (207) sequentially connected to the input branch migration domain (206) such that the output wobble domain (207) is sequentially interposed between a first portion of the input branch migration domain (206) and a second portion of the input branch migration domain (206).

18. The process of claim 11, wherein the output strand (201) further comprises a linker sequence (208) sequentially connected to the input branch migration domain (206) such that input branch migration domain (206) is sequentially interposed between the linker sequence (208) and the linker sequence (208).

19. The process of claim 11, wherein the gate prime strand (202) further comprises a transcription termination sequence (214) sequentially connected to the output toehold sequester domain (213) such that output toehold sequester domain (213) is sequentially interposed between the transcription termination sequence (214) and the substrate domain (211).

20. The process of claim 11, wherein the gate prime strand (202) further comprises a gate prime wobble domain (212) sequentially connected to the substrate domain (211) such that the gate prime wobble domain (212) is sequentially interposed between a first portion of the substrate domain (211) and a second portion of the substrate domain (211).
